SEQUENCE LISTING (66 sequences)

SEQ ID NO: 1 - DNA, 1932 bp, Arabidopsis thaliana
atgaagagattttatggagggcttcttgttgtatcaatgtgtatgttcttgacggtgtatagatatgtagacttgaacactcctgttgaaaagccttatattactgctgctgcttctgttgttgttactcctaacaccactcttcctatggaatggctgcggattactctccctgactttatgaaagaagcgaggaatactcaagaagcgatatctggtgatgatatcgctgttgtctcgggtttgtttgttgagcagaatgtgtctaaagaagagagagagcctttgcttacttggaaccgtttggaaagtcttgttgataatgcacagagtttagttaatggagttgatgctattaaggaagctggcattgtttgggagagtcttgtgtctgctgttgaagctaagaaactagttgatgttaatgaaaatcagacgaggaaaggaaaagaggagctttgtcctcagtttctaagcaaaatgaatgctactgaagctgatgggagtagtctgaagttgcaaattccttgtggtttgactcagggttcctccatcacagttattggcatcccagatggtcttgttggtagttttcggattgatctaacgggacaaccgcttccaggggagcctgatccacccatcattgtgcattataatgttaggcttcttggtgacaaatcgacggaagaccctgtgattgttcaaaacagctggacggcatctcaggactggggagctgaggaacgctgtccaaaatttgatcctgatatgaataagaaagtggatgacttggatgaatgcaacaagatggttggtggagaaattaaccgaacttcttcaactagcttgcagtccaacacttcaaggggagttccagtagccagggaagcatctaaacatgaaaaatactttcctttcaagcagggtttcttatcggttgctacacttagggtgggaacagagggaatgcagatgacagttgatgggaaacatataacttcatttgctttccgcgatacactggaaccgtggcttgttagtgaaatacggattacaggtgactttaggttaatatccattcttgccagcggtttgcccacatcagaagaatcagagcacgttgttgatctagaggcactaaaatcacctaccctttcaccattaaggccactggatctcgttattggtgttttctccactgcaaacaattttaaaagacggatggctgtgaggagaacatggatgcagtatgatgatgtaagatctggaagagttgcagtacgcttttttgttggccttcacaaaagtcctcttgttaacttggaactctggaacgaggctcggacttacggtgatgttcagctaatgccctttgttgattattacagtctcatcagttggaaaacactagccatctgcatcttcgggacagaggttgactcagccaagttcatcatgaaaacggatgatgacgcctttgttcgtgtagatgaagtgttactttctttatcaatgaccaacaacactcgcgggttaatatacggactgatcaattccgactctcaacctattcgaaaccctgatagcaaatggtacatcagttatgaggaatggcctgaagagaaatatccaccatgggcgcatggtccaggctacattgtatctcgtgacatagcagaatcggttggtaagcttttcaaagaaggaaacctaaagatgtttaagctagaagatgtggcaatggggatatggatagctgagctgacaaaacatggactcgagcctcattacgaaaacgatggaaggatcattagtgatggatgcaaggatggttatgtggttgctcattaccaaagccctgccgaaatgacttgcctttggcgtaaataccaagaaaccaaacgctctctttgctgccgcgaatggtaa

SEQ ID NO: 2 - protein, 643 aa, Arabidopsis thaliana
MKRFYGGLLVVSMCMFLTVYRYVDLNTPVEKPYITAAASVVVTPNTTLPMEWLRITLPDFMKEARNTQEAISGDDIAVVSGLFVEQNVSKEEREPLLTWNRLESLVDNAQSLVNGVDAIKEAGIVWESLVSAVEAKKLVDVNENQTRKGKEELCPQFLSKMNATEADGSSLKLQIPCGLTQGSSITVIGIPDGLVGSFRIDLTGQPLPGEPDPPIIVHYNVRLLGDKSTEDPVIVQNSWTASQDWGAEERCPKFDPDMNKKVDDLDECNKMVGGEINRTSSTSLQSNTSRGVPVAREASKHEKYFPFKQGFLSVATLRVGTEGMQMTVDGKHITSFAFRDTLEPWLVSEIRITGDFRLISILASGLPTSEESEHVVDLEALKSPTLSPLRPLDLVIGVFSTANNFKRRMAVRRTWMQYDDVRSGRVAVRFFVGLHKSPLVNLELWNEARTYGDVQLMPFVDYYSLISWKTLAICIFGTEVDSAKFIMKTDDDAFVRVDEVLLSLSMTNNTRGLIYGLINSDSQPIRNPDSKWYISYEEWPEEKYPPWAHGPGYIVSRDIAESVGKLFKEGNLKMFKLEDVAMGIWIAELTKHGLEPHYENDGRIISDGCKDGYVVAHYQSPAEMTCLWRKYQETKRSLCCREW

SEQ ID NO: 3 - DNA, 2467 bp, Arabidopsis thaliana
atgaagagattttatggagggcttcttgttgtatcaatgtgtatgttcttgacggtgtatagatatgtagacttgaacactcctgttgaaaagccttatattactgctgctgcttctgttgttgttactcctaacaccactcttcctatggaatggctgcggattactctccctgactttatgaaagaagcgaggaatactcaagaagcgatatctggtgatgatatcgctgttgtctcgggtttgtttgttgagcagaatgtgtctaaagaagagagagagcctttgcttacttggaaccgtttggaaagtcttgttgataatgcacagagtttagttaatggagttgatgctattaaggaagctggcattgtttgggagagtcttgtgtctgctgttgaagctaagaaactagttgatgttaatgaaaatcagacgaggaaaggaaaagaggagctttgtcctcagtttctaagcaaaatgaatgctactgaagctgatgggagtagtctgaagttgcaaattccttgtggtttgactcagggttcctccatcacagttattggcatcccagatggtcttgttggtagttttcggattgatctaacgggacaaccgcttccaggggagcctgatccacccatcattgtgcattataatgttaggcttcttggtgacaaatcgacggaagaccctgtgattgttcaaaacagctggacggcatctcaggactggggagctgaggaacgctgtccaaaatttgatcctgatatgaataagaaaggtgaatgcaagtatagtttcacccgcttacataaatgtagtcacttgatcctccatgaataagtctgtgttgacatgattataatacacagtggatgacttggatgaatgcaacaagatggttggtggagaaattaaccgaacttcttcaactagcttgcagtccaacacttcaaggggagttccagtagccagggaagcatctaaacatgaaaaatactttcctttcaagcagggtttcttatcggttgctacacttagggtgggaacagagggaatgcagatgacagttgatgggaaacatataacttcatttgctttccgcgatgtaatatcatcactcttcttatatatttttctactgctcttagcaattttccatcaaatgtttagtgtctcatatgggttcttcgtctttcagacactggaaccgtggcttgttagtgaaatacggattacaggtgactttaggttaatatccattcttgccagcggtttgcccacatcagaagaatcagagcacgttgttgatctagaggcactaaaatcacctaccctttcaccattaaggccactggatctcgttattggtgttttctccactgcaaacaattttaaaagacggatggctgtgaggagaacatggatgcagtatgatgatgtaagatctggaagagttgcagtacgcttttttgttggccttgtaagctttctccctctctgaaaatatcaggagtatgttggaattgcataattctaaatgatgattttctttgtggttggtaatgcagcacaaaagtcctcttgttaacttggaactctggaacgaggctcggacttacggtgatgttcagctaatgccctttgttgattattacagtctcatcagttggaaaacactagccatctgcatcttcggggtaataatatagttgtctttgtcatgtttcttcctagccacagttgagtagtctttactgttttgcctcactaacgtcatattttaaacagacagaggttgactcagccaagttcatcatgaaaacggatgatgacgcctttgttcgtgtagatgaagtgttactttctttatcaatgaccaacaacactcgcgggttaatatacggactgatcaattccgactctcaacctattcgaaaccctgatagcaaatggtacatcagttatgaggtacaacacaatctgacatagctatgtttgcaaatagactgatttctgattcatttaacgaagtttctactcaaaaaacaggaatggcctgaagagaaatatccaccatgggcgcatggtccaggctacattgtatctcgtgacatagcagaatcggttggtaagcttttcaaagaaggaaacctaaaggtattgtactactctatgctaactccattcaatcacatttccggattcacatagaaatggattgtgaaaatttttgaaatttgtgaatagatgtttaagctagaagatgtggcaatggggatatggatagctgagctgacaaaacatggactcgagcctcattacgaaaacgatggaaggatcattagtgatggatgcaaggatggttatgtggttgctcattaccaaagccctgccgaaatgacttgcctttggcgtaaataccaagaaaccaaacgctctctttgctgccgcgaatggtaaa

SEQ ID NO: 4 - protein, 192 aa, Arabidopsis thaliana
KRRMAVRRTWMQYDDVRSGRVAVRFFVGLHKSPLVNLELWNEARTYGDVQLMPFVDYYSLISWKTLAICIFGTEVDSAKFIMKTDDDAFVRVDEVLLSLSMTNNTRGLIYGLINSDSQPIRNPDSKWYISYEEWPEEKYPPWAHGPGYIVSRDIAESVGKLFKEGNLKMFKLEDVAMGIWIAELTKHGLEPH

SEQ ID NO: 5 - DNA, 34 bp, Artificial
  primer
tttaggtaccatgaagagattttatggagggctt

SEQ ID NO: 6 - DNA, 28 bp, Artificial
  primer
tataggatccttaccattcgcggcagca

SEQ ID NO: 7 - DNA, 287 bp, Arabidopsis thaliana
accttcgctttctctgctggtggaaggtgcttcgatactacgacaaagagcagcgaactagtaaagcctcggagatctgaatcccatttcaatcagcaaatctttccgtctctacttctctgtttgccggtgtaccggattctgattccggaacagacattgcttaattttgcttccctttggcctggattgattgaagttaaaaaatagctacattgtactgtaagattgaatattgatgatctcgccgcgcgatttctcgtttgagtgattttcggctgtgaaaa

SEQ ID NO: 8 - DNA, 219 bp, Arabidopsis thaliana
aacgtgaacgcacatatttcaaccattgcgttttggttcacgtgtccttccacaggtacataagaaaagagttcattctttggtcacaaaatatttgattttgtttgtttgttgtacaagtagactagatttgcagtttagttttacttttacgtgtgtatattacaaagatgtactaaaactttcaactgataaataacaataggagcatattttatc

SEQ ID NO: 9 - DNA, 2480 bp, Arabidopsis thaliana
atctattgaaggatccaatttccttgcatcgaaatactgcgggcacaacatgcatcattggcctagcttattagatccattacatattgtactattcattttttttccccggacatcttaggttaaagatagcatcatttttctaaagtaactgacattagcaacaccatgaattttgcgtgaagaaaaaaaatattgtagggccaaatctttatcattttctatagtatgaagattccaagactgaggttctcgcccgcacgagaagggcgagcttgaggtgtttatctgcaaggagatgggtgacgacgggtctaaagttgggtgggtttgtagatagaaggctgaggaagcttcggtaaccaccgctccatggagacgggtcattaaaaccatgttttgattactgttaaaagccatgttttgattaagcacaatgcatttcatgcataagcggttgaacattttttttttactctttagcaatatcgtgagccatttgtttttgttataacatgtacataagagaatgatgttgagctatcaaaaatcagtaaaatatagactcaacgatatataggataaagatgaggtaaaggttgatgaagaagaggataatgatcggttggtgaggaagaggataattaggttgatcaggtagaggatgatgatcattgatcacaatggatcgttggtaatagattgcccttcattaacagatgtggacttagttgacgtttgggaagactattgcttgatagagaatatgtcttgccttgatgtggcagacactggctttgttcctaatcccgataccaagtttcttagatctctttcttctgatgtatggatttgcgcttgaccaaatcaatggtagccattttatatatctctctctctctcgtattccttgttgtttcttcaactaatcttcgttttcctctatcataagtcgcgtgttgtagcgccattaacttctctctgctcataacgttttattttcaaacacaaaattccgtagactggttagaaccacttatgtttttgcttcaaaattctctaaaactgaaaactcttatgatcaacgaaaacacggtaacaagcgcatccatctaattttgatgaactcaacattataataaactgtactgataccaggacttcgagggtctccaaccttcctggaaccagtcgagttctattcccacatgcctgtcaactcatctagagatctttggttggaaagacaatggaagaagagaagatgagaaacaatttagtgacatacattctcgcaaactcaaagtgtttaaagacagtggaaatctccctcatagcagccttcaatcttaaggagaggcaaaaggagttagttaccatgcctaggatttcaacattatctaggcttctattccccatccaaatggaatgggagttcaatgaaacaactttttcattatatatattgtcatatacagtatatgctataactactcaagctgtcgtttacagattgcaatttttttgccttattaactcgaaaactctaattcagccaactttttactagtcgagatgaatttttttttaagcatatacgtacaacaaggctagattttttcatcatgagtaagtttaacaaagactgtgtgcttgcaaagtcatcatgagtaggttgttttgagtctcttatgtgaattaaacaaaacaaaaaaaacaacgtgaacatttcagtccaattttgtttgtctccctacgattcaaccagcaaaagaaaaaagcacataaaagcaaaaaatgggaatcaatatattgagacgttaattatgactggcatgtgtatgtatattgtatggtggtgcgttacaaatgaaagcaccctcatatctctcgctatcacgacttgggaacttgtttttaccccttcaagtttcgtttgccaacttttacttactgttttttttttaccagttactttattttactttttgcaatcaaatgtaactttgtttatttaaaaatgatgtttctagagggtcatcaaaaatatcagtgattaggcataattgtcaaatcttgataattcaaagatctcgtttatatgtgtttagtaatttagtcatagactcaaccaaaaaactttagtgatcaagtgatactttttttttttttttatcttcttccaaagcatgcaaacagaggaaatcaaaatataaatgtaatcttttaaaactacggttgtaattaaaattacttttttttttttttttgtcgtaaattactaaattagtattgaattaagttagaaactatttctgtgtattttcttagagtaccttgtgtataattttgtatgaaaatagctaaagaactatcggtaacttttcttcactttacagttttgttttttacctctacagtttttacaattttcgttcttgttttacagttaaaaaaaaatagaaagaaacatgaaacca

SEQ ID NO: 10 - protein, 507 aa, Arabidopsis thaliana
QKQGHGNDSSRAKEKQCPHFLNNMNSTELGNSSYKLQLPCGLTQGSSITIIGIPNGLLGNFRIDLTGEPLPGEPDPPIVLHYNVRLHGDKITEDPVIVQNSWTQAHDWGEEDRCPSPTPEKFDKVDDLEQCNKIVGKNISQRHPAGMHSHTSRQSSTMDEQSVNRKYFPFKQGYPFVATLRVGSEGIQMTVDGKHITSFAFRETLEPWLVSEIKISGDLKLISILASGLPTSEDSEHIIDLESLKSSPISAQTLLYLFIGVFSTANNFKRRMAVRRTWMQYNAVRSNTTAVRFFVGLHKSTVVNEELWREARTYGDVQLMPFVDYYSLITWKSLAICIFGTQVSAKFVMKTDDDAFVRVDEVLDSLHRINADHGLLYGLINLDSRPHRNTDSKWYISPEEWSEGTYPPWAHGPGYVVSHDIARTVSKKFRENHLKMFKLEDVAMGIWIADMKKEGLEVRYENEVRVYPEGCKDGYVVAHYQGPREMLCLWQKLQVDKRAKCCGDSRK

SEQ ID NO: 11 - DNA, 31 bp, Artificial
  primer
tatactgcagaagccttatattactgctgct

SEQ ID NO: 12 - DNA, 33 bp, Artificial
  primer
taaaggtaccttaccattcgcggcagcaaagag

SEQ ID NO: 13 - DNA, 23 bp, Artificial
  primer
aaggccactggatctcgttattg

SEQ ID NO: 14 - DNA, 25 bp, Artificial
  primer
ttaccaaccgattctgctatgtcac

SEQ ID NO: 15 - DNA, 23 bp, Artificial
  primer
ggtgctcggaattttcgtctaca

SEQ ID NO: 16 - DNA, 24 bp, Artificial
  primer
tcattctctttgcaactggctcta

SEQ ID NO: 17 - DNA, 22 bp, Artificial
  primer
attgccgtagatgaaagactga

SEQ ID NO: 18 - DNA, 31 bp, Artificial
  primer
tttatctagatggcttcaaaggtctcctgcc

SEQ ID NO: 19 - DNA, 31 bp, Artificial
  primer
ttctggatcctaacatctcagatgcttcttg

SEQ ID NO: 20 - DNA, 30 bp, Artificial
  primer
atcagggatccactgcacggtatgctcctc

SEQ ID NO: 21 - DNA, 30 bp, Artificial
  primer
atcgtggtacctagctgcgtctgcaaaaag

SEQ ID NO: 22 - DNA, 34 bp, Artificial
  primer
tatatctagaaaagtcctcttgttaacttggaac

SEQ ID NO: 23 - DNA, 32 bp, Artificial
  primer
tataagatcttaaacatctttaggtttccttc

SEQ ID NO: 24 - DNA, 33 bp, Artificial
  primer
tataggtaccttaaacatctttaggtttccttc

SEQ ID NO: 25 - DNA, 41 bp, Artificial
  primer
tatagaattcggatccaaaagtcctcttgttaacttggaac

SEQ ID NO: 26 - DNA, 422 bp, Arabidopsis thaliana
aaaagtcctcttgttaacttggaactctggaacgaggctcggacttacggtgatgttcagctaatgccctttgttgattattacagtctcatcagttggaaaacactagccatctgcatcttcgggacagaggttgactcagccaagttcatcatgaaaacggatgatgacgcctttgttcgtgtagatgaagtgttactttctttatcaatgaccaacaacactcgcgggttaatatacggactgatcaattccgactctcaacctattcgaaaccctgatagcaaatggtacatcagttatgaggaatggcctgaagagaaatatccaccatgggcgcatggtccaggctacattgtatctcgtgacatagcagaatcggttggtaagcttttcaaagaaggaaacctaaagatgtttaa

SEQ ID NO: 27 - DNA, 422 bp, Arabidopsis thaliana
ttaaacatctttaggtttccttctttgaaaagcttaccaaccgattctgctatgtcacgagatacaatgtagcctggaccatgcgcccatggtggatatttctcttcaggccattcctcataactgatgtaccatttgctatcagggtttcgaataggttgagagtcggaattgatcagtccgtatattaacccgcgagtgttgttggtcattgataaagaaagtaacacttcatctacacgaacaaaggcgtcatcatccgttttcatgatgaacttggctgagtcaacctctgtcccgaagatgcagatggctagtgttttccaactgatgagactgtaataatcaacaaagggcattagctgaacatcaccgtaagtccgagcctcgttccagagttccaagttaacaagaggactttt

SEQ ID NO: 28 - protein, 557 aa, Arabidopsis thaliana
VSDLFIKRNLSAQEQQSLSTWYQLKRLVTHDQVLPNAIEAVKEASVAWNNLMSAVEREKLDANDSSVKTGKQKQCPHFLSKTNATEIDASGFKLRFPCGLTQGSSITIIGIPNGLLGNFRIDLTGEPLPGEPDPPVILHYNVRLHGDKITEDPVIVQNTWTIAHDWGEEERCPLPSDEKSKKVDELEQCNEMVGNVITRHVIATNKSSLAQDGSKSRKYFPFKQGYLSVATLRVGSEGIQMAVDGKHITSFAFRETLEPWLVSEVRISGDIKLISVVASGLPTSEDSEHISDLEALKAVPLPPRKRLDLFVGVFSTANNFKRRMAVRRTWMQYDAVRSGQVAVRFFVGLHKNQMVNEELWNEARTYMDIQLMPFVDYYSLIAWKTIAICVFGTEVISAKFVMKTDDDAFVRVDEILSSMQRINVTRGLLYGLINSDSHPHRSPDSKWFISPEEWPEETYPPWAHGPGYVVSSDIAKTISSKQRKGRLKMFKLEDVAMGIWISEIKKKGLEVKYEKEERIFNEGCRDGYVIAHYQGPREMLCLWQKIEEKKRALCCGE

SEQ ID NO: 29 - protein, 548 aa, Arabidopsis thaliana
SAQEQQSLSTWYQLKRLVTHDRVLPNAIEAVKEASIAWNNLMSAVEREKLDANDTSVKTGKQKQCPHFLSKTNATEIDASGFKLRFPCGLTQGSSITIIGIPNGLLGNFRIDLTGEPLPGEPDPPVILHYNVRLHGDKITEDPVIVQNTWTIAHDWGEEERCPLPSDEKSKKVDELEQCNEMVGNIMATRHVIATNKSSLAQDRSKSRKYFPFKQGYLSVATLRVGSEGIQMTVDGKHITSFSFRETLEPWLVSEVRISGDIKLISVVASGLPTSEDSEHISDLEALKAAPLPPRKRLDLFVGVFSTANNFKRRMAVRRTWMQYDAVRSGQVAVRFFVGLHKNQMVNEELWNEARTYMDIQLMPFVDYYSLIAWKTIAICVFGTEVVSAKFVMKTDDDAFVRVDEILSSMQRINVTRGLLYGLINSDSHPHRSPDSKWFISPEEWPEETYPPWAHGPGYVVSSDIAKQSAQNRKKGRLKMFKLEDVAMGIWISEMNKKGLEVKYEKEERIFNEGCRDGYVIAHYQGPREMLCLWQKIEEKKRALCWGE

SEQ ID NO: 30 - protein, 637 aa, Arabidopsis thaliana
MKKWHGGFVIVSLLIILMLRYVILDRLLAEKSLQYVFQQNRTAELHWLDVPNPPAIQNPQSSSEVISTRLLASNLSITRNLSDTELQSLHSWNHLKHLLTHAHILPDGVEAIKEAGVAWRELNTALAYDDSVVSVNGSIQQKDKGKQCPYSVRRMNATRLGDRFALKLPCGLIQGSSITIIGTPGGLLANFKIELTGAAVPGEPDPPIVLHYNVRLLGDKLTEDPVIVQNTWTIADDWGSENRCPSPDSDAKDSAKVDDLEKCSSMVGEDQKKILPSKLHSHVLTMPPARKKKAESRKYFPFRQGYLAVAILRIGAHGIHMTVDGKHITSFAFREDLEPGFVGEVRIAGDIKLLSVIASGLPTTEYFEHVTDLEILKAPPVAMDKSVDLFIGVFSTANNFKRRMAVRRTWMQYDAVRSGKVAVRFFVGLHKNEVVNEELWNEARTYGDIQLMPFVDYYSQILWKTIAICIYGTNVLSAKYVMKTDDDAFVRVDEILSSLDRTNISHGLLYGRVNSDSQPHRDPYSKWYITPEEWPEENYPPWAHGPGYIVSQDIAKEVYRKHKNGELKMFKLEDVAMGIWINEMKKDGLDVKYENDGRILVEGCEDGYVIAHYQEPRDMMCLWDKFQKTKRGTCCKE

SEQ ID NO: 31 - protein, 637 aa, Oryza sativa
MKKWHGGFVIVSLFIILMLRYVILDSPLAEKSLQYVFQQNNTTPLHWLDVPNPPAVQNPQNISQVISTELLASNLSITRNFSDRELQSLHSWNHLKDLVSHAHILPDGVEAIKEAGVAWRELNAALEYDESAVSVNGSTHQKSKEKQCPYSIRRMNATRSGDRFVLKIPCGLIQGSSITIIGTPGGLLGSFKIELTGATVPGEPDPPIVLHYNVRLLGDKLTEDPVIVQNTWTIADDWGSEDRCPSPGSDAKDIAKVDELEKCGSMVGNDQKQAWATKLKSNVSSIQPAWKKNTEPKKYFPFRQGYLAIAILRVGAHGIHMTVDGKHVTSFAFREDLEPGFVGEVRIAGDIKLLSVIASGLPTTEDFEHVTDLEILKAPPVPMDKPLDLFIGIFSTANNFKRRMAVRRTWMQYDAVRSGKAAVRFFVGLHKNEVVNEELWNEARTYGDIQLMPFVDYYSLILWKTIAICIYGTNVLSAKYVMKTDDDAFVRVDEILLSLDRVNISHGLLYGRVNSDSQPHRDPYSKWYITPEEWPEESYPPWAHGPGYIVSQDIAKEVYRKHKRGELKMFKLEDVAMGIWINEMKKDGLDVKYENDGRILVEGCEEGYVVAHYQEPRDMMCLWDKFLKTKRGTCCKE

SEQ ID NO: 32 - DNA, 1914 bp, Oryza sativa
atgaagaaatggcacggtggttttgtcatagtatctttgttcattatattgatgctaagatatgttatattggatagtccacttgctgaaaaatctcttcagtatgtcttccaacagaataacacaaccccactacattggctggatgttccaaacccgcctgcagttcagaatccacagaacatctctcaagtaatatcaactgaattgttagcttcaaacctttccataacaagaaatttctccgatagagaactgcagtcattgcattcttggaatcatttaaaagacctagtaagtcatgcgcacatcttaccagatggagtagaagcaatcaaggaagctggagtagcatggagggagctaaatgcagcccttgaatacgatgaatctgctgtttctgtcaatggtagcactcatcaaaaaagtaaagagaagcaatgcccatattccattcgaagaatgaatgctacaagatcaggtgatagatttgtcttaaagattccttgtggactgattcagggctcttcaataactattatcggtactcctggtggtcttcttggtagttttaagatagaattaactggggctactgtacctggtgaaccagaccctccaattgtgcttcattacaatgtccgtcttcttggtgataaacttacagaagatcctgtaatagtccaaaatacatggactatagctgacgactggggttctgaagatcgttgcccatctcctggttcagatgctaaggacattgcaaaagtggatgagttagaaaaatgcggtagcatggtgggcaatgatcaaaaacaagcatgggccactaagttaaagtccaatgtttcaagcatacaacctgcctggaaaaagaacacagaacctaagaaatattttcccttcagacaaggatatcttgctatagcaattcttcgtgttggagcacatgggatccatatgactgtagatgggaaacatgtcacttcatttgcctttcgggaggatttggagcctggatttgttggtgaagtaagaattgcaggagatattaaattgttgtctgtgatagcaagtggtttgcctacaacagaggactttgaacatgtgactgacttagaaattttgaaggctccaccagttcccatggacaaacccctggatctttttattgggatcttttctacagctaacaatttcaagcgcagaatggcagttcgcagaacttggatgcagtatgatgctgtccgctcaggaaaggctgcagttcgattctttgttggcctgcataaaaatgaggtggtgaatgaggaactttggaatgaagcacgaacatatggagacatccaattgatgccctttgttgattattatagcctgattctatggaagactatagccatctgcatctatgggacaaacgtcttgtcagccaagtatgtgatgaaaacagatgatgatgcttttgtccgtgtggatgagatacttttatctttagatcgcgtgaacatcagtcatgggttactgtacggtcgtgtcaattctgattctcagcctcatcgagacccatatagcaagtggtacataaccccagaggaatggcctgaggagagttatcctccatgggcacatggtccgggatacattgtatcacaagacatagcaaaagaagtttacaggaaacacaagagaggagaacttaagatgttcaaactagaagatgtcgcaatgggaatatggatcaatgagatgaagaaggatgggttggatgtaaagtatgaaaatgatgggaggattttggtcgaaggctgtgaggaaggatatgtggttgcacattaccaggagccaagggacatgatgtgcctatgggacaaatttctgaaaacaaaacgaggaacatgttgcaaggagtaa

SEQ ID NO: 33 - protein, 621 aa, Oryza sativa
MWVTKRLGITVLIVLFPLLIVHHLIVNSPVSGPSRYQVIHSNLLGWLSDSLGNSVAQNPDNTPVEVIPADASASNSSDSGNSSLEGFQWLNTWNHMKQLTNISDGLPHANEAIDNARTAWENLTISVHNSTSKQIKKERQCPYSIHRMNASKPDTGDFTIDIPCGLIVGSSVTIIGTPGSLSGNFRIDLVGTELPGGSGKPIVLHYDVRLTSDELTGGPVIVQNAFTASNGWGYEDRCPCSNCNNATQVDDLERCNSMVGREEKRAINSKQHLNAKKDEHPSTYFPFKQGHLAISTLRIGLEGIHMTVDGKHVTSFPYKAGLEAWFVTEVGVSGDFKLVSAIASGLPTSEDLENSFDLAMLKSSPIPEGKDVDLLIGIFSTANNFKRRMAIRRTWMQYDAVREGAVVVRFFVGLHTNLIVNKELWNEARTYGDIQVLPFVDYYSLITWKTLAICIYGTGAVSAKYLMKTDDDAFVRVDEIHSSVKQLNVSHGLLYGRINSDSGPHRNPESKWYISPEEWPEEKYPPWAHGPGYVVSQDIAKEINSWYETSHLKMFKLEDVAMGIWIAEMKKGGLPVQYKTDERINSDGCNDGCIVAHYQEPRHMLCMWEKLLRTNQATCCN

SEQ ID NO: 34 - DNA, 1866 bp, Oryza sativa
atgtgggtgacaaagcgactcggaattacggttctaatcgtcctcttccctctgctgattgtgcatcatctgatagtgaatagcccagtgtcagggccatctcgataccaagtcatacattctaatctccttggttggttgagcgactcattgggaaattcagtggctcaaaatccagataatacaccagtagaagtgataccagcagatgcttcagcttcaaacagctcggattccggaaattcctctcttgaaggatttcagtggttaaatacatggaatcacatgaagcagttgaccaacatctccgatggccttcctcacgcaaatgaggctatagacaatgcaagaactgcatgggaaaacctgacgatatcagttcacaattcaacctctaaacagattaagaaagagaggcagtgtccatattctatccacagaatgaatgcctccaagccagacactggagattttaccatcgatataccttgtgggctgattgtaggttcttcagtaactattataggcactccaggtagcctctccggtaacttcaggattgatcttgttgggacagaattgccagggggatctgggaaacccatcgtactgcactatgatgttcgtctaacaagtgatgaacttactggaggtccagtcatagtgcagaatgccttcactgcaagtaatggctggggttatgaggatagatgtccctgcagtaactgtaataatgcaactcaagtggatgatttggagcgatgtaattccatggtgggtagagaagagaagagagctataaactcaaaacagcacctgaatgccaagaaagatgaacacccaagcacatattttccatttaagcagggacaccttgctatctcaactcttcggatagggctagaagggattcacatgacagttgatggaaaacatgttacctcatttccatataaagcgggcttggaagcatggttcgttactgaagtaggagtatctggtgatttcaagctagtaagtgcaattgcaagtggcctgcccacatcagaagacctggagaacagcttcgatcttgcaatgttgaagtcgtcacctattccagagggaaaagatgtggaccttttgattggtatcttttcaacagcaaacaacttcaaacgaaggatggcgatccgacggacatggatgcaatatgatgcggtgcgcgaaggagcagtagtggtccgttttttcgttggcctgcatacaaacctcattgtaaacaaggaactctggaatgaagcacgcacatatggcgacattcaagtgctgccctttgttgattactacagtctgataacatggaagacactggcaatatgcatctacgggactggtgctgtatcagcgaaatatctgatgaaaacagacgatgatgcttttgtccgtgtggacgagatacactcctcagtaaaacaactgaatgtcagtcacggcctactttatggtcgtatcaattctgactcaggtcctcacagaaatcctgagagcaaatggtacataagcccagaggaatggcctgaagagaaatacccaccatgggcacacggaccaggatatgtggtatcacaagacattgcaaaggaaatcaacagttggtatgaaacaagtcatttgaagatgttcaaactagaagatgtagcaatgggtatatggattgctgaaatgaagaagggtgggttacctgtccaatacaaaacagatgaaagaattaacagtgatggctgcaatgatgggtgcatcgttgctcactaccaagaaccaaggcacatgctatgcatgtgggagaaactcctaaggacaaatcaagcaacgtgctgcaactaa

SEQ ID NO: 35 - protein, 632 aa, Populus trichocarpa
MKKWYGGVLVASLFMLLILRYGLLKNPIGGNILLNPSSNASKPLEWVHPAVPPAVQNPETSSQVFSTDTIVSSLFAPRNISNEEHKSLQTWNLLKHLIDHAQVFANGVEAIKEAGNAWSSLMASAEEERLSYTNESSSRKVKEKQCPHFLNKMNATELDNSGYKLWLPCGLTQGSSITIISIPDGLLGNFRIDLTGEALPGEPDPPIILHYNVRLHGDKITEDPVIVQNTWTVAHDWGEEERCPSPSPEKIKKVDELDQCNKMVGRNDTRVTSMHSDHSRRSSLQEGTKARRYFPFKQGQLSVATLRVGMEGIQMTVDGKHITSFAYRETLEPWLVSEVRISGDLNLISVVASGLPTSEDSEHAVDLEVLKSAPLSPKKTLDLFIGVFSTANNFKRRMAVRRTWMQYAAVRSGAVAVRFFVGLHKNQIVNEELWNEARTYGDIQLMPFVDYYNLITFKTLAICIFGTEVASAKYVMKTDDDAFVRVDEVLASLKRIKVSHGLLYGLINSDSRPHRSTESKWYISPEEWSEETYPPWAHGPGYVVSRDIAKAVYKRYKEGRLKMFKLEDVAMGIWIAEMKKEGLEVKYEMEGRVHNEGCRDGYVVAHYQAPREMLCLWQKLQEGNVARCCGDR

SEQ ID NO: 36 - DNA, 1899 bp, Populus trichocarpa
atgaagaagtggtacggtggtgttctggttgcatccttgttcatgttactgatccttagatatggtctcctgaaaaatccaatcggaggaaatattctgttgaatccctcctccaatgcaagcaagcctcttgaatgggtgcatcctgctgttccacctgcagttcaaaatccagagacttcttctcaagttttttctacagataccatagtctccagtctgtttgctccgagaaatatttccaatgaagagcacaagtctctgcagacatggaatctactgaaacacttgattgatcatgctcaggttttcgcaaatggagtagaagctattaaggaagctggaaatgcgtggagcagcctgatggcttcagcagaagaggaaagacttagttatacaaatgaaagttcaagtaggaaagtgaaagagaaacaatgtcctcactttcttaacaaaatgaatgccacagagcttgataatagtggttataaattgtggcttccttgtggcttgactcagggttcgtccatcacaattatcagcattccagatggtcttctcggtaattttcggatcgacttaaccggggaagcacttccaggggagcctgatccacccatcattttacattacaatgttaggcttcatggtgataagattacagaggatccagtaatagtccaaaacacctggactgtggctcatgactggggtgaagaggagcgttgtccttctccatctcctgaaaaaattaagaaagtggacgagttggatcaatgtaataagatggtgggcagaaacgatactcgggtgactagcatgcattctgatcattcgagaaggtcatcacttcaggaaggaactaaagcaagaagatattttccatttaagcaaggtcagctatctgtcgcaacgcttagagtgggaatggaaggaatccagatgactgttgatggaaagcacataacatcctttgcatatcgtgaaactttggagccatggctagttagtgaagttaggatttctggggacctaaatctaatttcagttgtggctagtggtctacctacatcagaggattcagagcatgcagttgatctagaagtactcaaatcagctcctctctctccaaaaaaaacactggatctctttattggtgtcttttctacggcaaacaattttaagcggaggatggctgttcgaagaacatggatgcagtatgctgcagtgcgatcaggggcagttgcagtgcgcttttttgttggtctgcataaaaatcaaatagtgaatgaggaactctggaatgaagcacggacatatggagacatacagctgatgccttttgttgactactataacctcatcacctttaaaactttagctatctgcatatttgggacagaggttgcatcagctaagtatgtcatgaagacagatgacgatgcgtttgttcgtgtggatgaagtgttagcttctttaaagaggatcaaagtgagtcatgggttgctctatggattaatcaattcagactctcggcctcataggagtactgaaagcaagtggtatattagccctgaggaatggtctgaagagacctatcccccctgggcacatggtcctggttacgttgtgtctcgtgacattgccaaggcagtttacaagagatacaaagaaggccgtttaaagatgtttaagctagaggatgtagcaatgggtatctggattgctgagatgaaaaaggagggtttagaagtcaaatatgagatggaagggagggtccataatgaagggtgcagggatggctatgttgtagcccactaccaagctcctagggaaatgctctgtctgtggcagaaacttcaagaagggaatgttgctagatgctgcggtgatcgataa

SEQ ID NO: 37 - protein, 637 aa, Zea mays; identical to SEQ ID NO: 30

SEQ ID NO: 38 - DNA, 1914 bp, Zea mays
atgaagaaatggcatggaggctttgtcattgtatctttgctcatcatcttgatgttaagatatgtgatattggataggctacttgcagaaaaatcgcttcagtatgttttccagcaaaacaggacagcagaattacattggttggatgttccaaacccacctgcaatccagaatccacagagttcttctgaagtaatatcaaccagactcttggcatccaacctttccattaccagaaatctctctgatacagaacttcaatctttacattcttggaatcatttgaaacaccttttaacgcatgcccatatcctaccagatggagtagaggcaatcaaggaagctggagttgcatggagggaactcaatacagcccttgcatatgatgattcagttgtttctgttaatggcagcattcagcagaaggacaaaggaaagcaatgcccttattctgtccgacggatgaatgccacaagattaggggataggtttgctttaaaacttccatgtggattgattcagggctcttcaataacaattattggtactcctggtggtcttctggctaattttaagatagagttaactggagctgcagttcctggtgaaccagaccctccaattgtccttcattataatgtccgccttcttggtgataaactcacagaagatcctgtaatagtccaaaatacatggacgatagctgatgattggggttctgaaaaccgttgcccatctcctgattcagatgctaaggacagtgcaaaagtggatgatctagaaaagtgtagtagcatggtaggtgaggaccagaagaaaatccttccctctaagttacattcacatgttttgaccatgccacctgcaaggaagaagaaagcagaatctagaaagtattttccattcaggcagggatatcttgctgtagcaattcttcgtatcggagcacatgggatccatatgactgtagatggcaaacatatcacttcgtttgcatttcgagaagatctggaaccagggtttgttggtgaagtaagaattgcaggagacattaaattgctatctgtgatagcaagtggcctgccaacaacagagtacttcgagcatgtcactgatttggaaattttgaaggctccacctgttgccatggataaatctgttgatcttttcattggggtcttctctacagcaaataatttcaaacgccgaatggcagttcgcagaacttggatgcagtatgatgctgtgcgctcaggaaaagttgcagttcgattctttgttggcctgcacaaaaacgaggtggtcaatgaggaactctggaatgaagcacgaacatatggagacatccagttgatgccgtttgtggattactatagccagattctttggaagaccatagcgatctgcatctatgggacaaatgtcctgtcagctaagtatgtgatgaaaactgatgatgatgcttttgttcgagtagatgaaatactttcttctctcgaccgaaccaatatcagccatggactgttgtatggtcgtgtcaattctgattcccagcctcatcgagatccctatagcaagtggtacataacccctgaggaatggcctgaggagaattatcctccatgggcgcatggtccaggatatattgtttcacaggacatagctaaagaagtttacaggaaacacaagaatggggaattaaagatgttcaagctggaggatgtcgcgatgggaatatggatcaatgagatgaagaaggatggcttggatgtcaagtatgagaatgatgggaggatcttggttgaaggatgcgaggatgggtatgtgattgctcactaccaagaaccaagggatatgatgtgcctctgggataagtttcagaaaacaaaacgaggaacatgttgcaaagaataa

SEQ ID NO: 39 - protein, 637 aa, Zea mays
MKKWHGGFVIVSLFIILMLRYVILDRLLAEKSLQYVFQQNRTAELHWLDVPNPPAIQNPQSSSEVISTRLLASNLSITRNLSDTELQSLHSWNHLKHLLTHAHILPDGVEAIKEAGVAWRELNTALAYDDSVVSVNGSIQQKDKGKQCPYSVRRMNATRLGDRFALKLPCGLIQGSSITIIGTPGGLLANFKIELTGAAVPGEPDPPIVLHYNVRLLGDKLTEDPVIVQNTWTIADDWGSENRCPSPDSDAKDSAKVDDLEKCSSMVGEDQKEILPSKLHSHVLTMPPARKKKAESRKYFPFRQGYLAVAILRIGAHGIHMTVDGKHITSFAFREDLEPGFVGEVRIAGDIKLLSVIAGGLPTTEYFEHVTDLEILKAPPVAMDKSVDLFIGVFSTANNFKRRMAVRRTWMQYDAVRSGKVAVRFFVGLHKNEVVNEELWNEARTYGDIQLMPFVDYYSLILWKTIAICIYGTNVLSAKYVMKTDDDAFVRVDEILSSLDRTNISHGLLYGRVNSDSQPHRDPYSKWYITPEEWPEENYPPWAHGPGYIVSQDIAKEVYRKHKNGELKMFKLEDVAMGIWINEMKKDGLDVKYENDGRILVEGCEDGYVIAHYQEPRDMMCLWDKFQKTKRGTCCKE

SEQ ID NO: 40 - DNA, 1914 bp, Zea mays
atgaagaaatggcatggaggctttgtcattgtatctttgttcatcatcttgatgttaagatatgtgatattggataggctacttgcagaaaaatcgcttcagtatgttttccagcaaaacaggacagcagaattacattggttggatgttccaaacccacctgcaatccagaatccacagagttcttctgaagtaatatcaaccagactcttggcatccaacctttccattaccagaaatctctctgatacagaacttcaatctttacattcttggaatcatttgaaacaccttttaacgcatgcccatatcctaccagatggagtagaggcaatcaaggaagctggagttgcatggagggaactcaatacagcccttgcatatgatgattcagttgtttctgttaatggcagcattcagcagaaggacaaaggaaagcaatgcccttattctgtccgacggatgaatgccacaagattgggggataggtttgctttaaaacttccatgtggattgattcagggctcttcaataactattattggtactcctggcggtcttctggctaattttaagatagagttaactggagctgcagttcctggtgaaccagaccctccaattgtccttcattataatgtccgccttcttggtgataaactcacagaagatcctgtaatagtccaaaatacatggacgatagctgatgattggggttctgaaaatcgttgcccatctcctgattcagatgctaaggacagtgcaaaagtggatgatctagaaaagtgtagtagcatggtaggtgaggaccagaaggaaatccttccctctaagttacattcacatgttttgaccatgccacctgcaaggaagaagaaagcagaatctagaaagtattttccattcaggcagggatatcttgctgtagcaattcttcgtatcggagcacatgggatccatatgactgtagatggcaaacatatcacttcgtttgcatttcgagaagatctggaaccagggtttgttggtgaagtaagaattgcaggagacattaaattgctatctgtgatagcaggtggcctgccaacaacagagtacttcgagcatgtcactgatttggaaattttgaaggctccacctgttgccatggataaatctgttgatcttttcattggggtcttctctacagcaaataatttcaaacgccgaatggcagttcgcagaacttggatgcagtatgatgctgtgcgctcaggaaaagttgcagttcgattctttgttggcctgcacaaaaacgaggtggtcaatgaggaactctggaatgaagcacgaacatatggagacatccagttgatgccatttgtggattactatagcctgattctttggaagaccatagcgatctgcatctatgggacaaatgtcctgtcagctaagtatgtgatgaaaactgatgatgatgcttttgttcgagtagatgaaatactttcttctctcgaccgaaccaatatcagccatggactgttgtatggtcgtgtcaattctgattcccagcctcatcgagatccctatagcaagtggtacataacccctgaggaatggcctgaggagaattatcctccatgggcgcatggtccaggatatattgtttcacaggacatagctaaagaagtttacaggaaacacaagaatggggaattaaagatgttcaagctggaggatgtcgcgatgggaatatggatcaatgagatgaagaaggatggcttggatgtcaagtatgagaatgatgggaggatcttggttgaaggatgcgaggatgggtatgtgattgctcactaccaagaaccaagggatatgatgtgcctctgggataagtttcagaaaacaaaacgaggaacatgttgcaaagaataa

SEQ ID NO: 41 - protein, 636 aa, Zea mays
MKKWHGGFVIVSLFIILMLRYVILDNPLVENSLQYVFQQNRTAELHWLDVPNPPAIQNLQISSEVISTGLLASNLSITRNLSDRELQSLHSWNHLKQLLSHVHILPDGVEAIKEAGVAWRELNTALAYDDAVSVNGSIQQKDKGKQCPYSIRRMNATRLGDRFALKLPCGLIQGSSITIIGTPGGLLGNFKIELTGAAVPGEPDPPIVLHYNVRLLGDKLTEDPVIVQNTWTISDDWGSENRCPSPDSDAKDTTKVDDLEKCSSMVGEDQKEILPSKFHSNVSAMPPASKKKAEPRKYFPFRQGYLAVAILRIGAHGIHMTVDGKHITSFAFREDLEPGFVGEVRIAGDIKLLSVIASGLPTTEDFEHVTDLEILKAPPVPMDKPVDLFIGVFSTANNFKRRMAVRRTWMQYDSVRSGKVTVRFFVGLHKNELVNEELWNEARTYGDIQLMPFVDYYSLILWKTIAICIYGTNVLSAKYVMKTDDDAFVRVDEILSSLDRTNISHGLLYGRVNSDSQPHRDPYSKWYITPEEWPEENYPPWAHGPGYIVSKDIAKEVYRKHKSGELKMFKLEDVAMGIWINEMKKDGLDVKYENDGRILVEGCEDGYVIAHYQEPRDMMCLWDQFQKTKRGTCCKE

SEQ ID NO: 42 - DNA, 1911 bp, Zea mays
atgaagaaatggcatggaggctttgtcattgtatctttgttcatcatcttgatgttaagatatgtgatattggataacccacttgtagaaaactcgcttcagtatgttttccagcaaaacaggacggcagaattacattggttggatgttccaaacccacctgcaatccagaatctacagatttcttctgaagtaatatcaactggactcttggcttccaacctttccattaccagaaatctctctgatagagaacttcaatctttacattcttggaatcatttgaaacagctattatctcatgttcatatcctaccagatggagtagaggcaatcaaggaagctggagttgcatggagggaactcaatacagcccttgcatatgatgatgcagtttctgttaatggcagcattcagcagaaggacaaaggaaagcaatgcccttattctatcagaaggatgaatgccacaagattaggggataggtttgctttaaaacttccctgtggactgattcagggctcttcaataactattattggtacacctggtggtcttcttggtaattttaagatagagttaactggggctgcagttcctggtgaaccagaccctccaattgtccttcattataatgtccgccttcttggtgataaactcacagaagatcctgtaatagtccaaaatacatggacaatatctgatgattggggttctgaaaaccgttgcccatctcctgattcagatgctaaggacactacaaaagtggatgatctagaaaaatgtagtagcatggtaggtgaggaccagaaagaaatccttccctctaagtttcattcaaatgtttcggccatgccacctgcaagtaagaagaaagcagaacctagaaagtattttccattcagacagggatatcttgccgtagcaattcttcggatcggagcacatgggatccatatgactgtagatggcaaacatatcacttcttttgcattccgagaggatttggaaccagggtttgtaggtgaagtaagaattgcaggagacattaaattgttatctgtgatagcaagtggcctgcctacaacagaggacttcgagcatgtcactgacttagaaatattgaaggctccacctgtgcccatggataaaccggttgatcttttcattggggtcttctctacagcaaataatttcaaacgccgaatggcagttcgcagaacttggatgcagtatgattctgttcgatcaggaaaagttacagttcgattctttgttggcctgcacaaaaatgagttggtcaatgaggaactctggaatgaagcacgaacatatggagacatccagttgatgccatttgtggattactatagcctgattctttggaagaccatagcgatctgcatctatgggacaaatgtcctgtcagctaagtatgtgatgaaaactgatgatgatgcttttgttcgagtagatgaaatactttcttctctcgaccgaaccaatatcagccatggactgttgtatggtcgtgtcaattctgattcccagcctcatcgagatccctatagcaagtggtacataacccctgaggaatggcctgaggagaattatcctccctgggcgcatggaccaggatatattgtttcaaaggacatagctaaagaagtttacaggaaacacaagagtggggagttaaagatgttcaagctggaggatgtcgcaatgggaatatggatcaatgagatgaagaaggatggcttggatgtcaagtatgagaatgatgggaggattttggttgaaggatgcgaggatggctatgtgattgctcattaccaagaaccaagggatatgatgtgcctctgggatcagtttcagaaaacaaaacgaggaacatgttgcaaagaataa

SEQ ID NO: 43 - protein, 557 aa, Nicotiana tabacum; identical to SEQ ID NO: 28

SEQ ID NO: 44 - DNA, 1674 bp, Nicotia tabacum
gtctctgatctctttatcaagaggaacctgtcagctcaggagcaacaatctttgtctacgtggtatcagttgaaacgcttagttactcatgatcaagtcttacctaatgctatagaggcagttaaggaagccagtgttgcatggaacaacctcatgagtgcagtagagagggaaaaacttgatgcaaatgatagttcagttaagaccgggaaacagaaacaatgccctcattttctcagcaaaaccaatgctacagaaattgatgccagtggctttaagttgcggtttccttgtggtctgactcagggttcttccatcacaataattggcattccaaatggtcttcttgggaattttcggatagatttgactggtgaaccacttccaggtgaaccagatcctcctgttattctgcattacaatgttaggctccatggtgataaaataactgaggatcccgtaattgtacaaaacacctggacaattgcacatgactggggtgaagaggagcgctgtcccttaccatcggatgaaaagagtaagaaagtggatgaattggagcaatgcaatgagatggtgggaaatgtcataactcgacatgtcattgcaacaaataaatctagcctggctcaggacgggtctaaatcaagaaaatattttcctttcaagcaaggatatctctctgttgccactctgagagtaggatctgaaggaattcagatggcagttgatggaaaacacataacatcttttgctttccgtgaaactttggaaccgtggcttgtaagtgaagtgaggatatccggagacataaaattaatttctgttgtcgcgagtggtttgccaacatctgaggattcagagcatataagtgacttggaagctctaaaagcagttcctcttcctcctcggaaaagactagatctctttgttggtgtattttctaccgcaaataattttaagcgcagaatggctgtccgtagaacttggatgcaatatgatgcagtgcgatctggacaagttgcggtgcggttttttgttggcttgcataaaaaccaaatggtgaatgaagagctctggaatgaggctaggacatatatggacatccagctgatgccttttgttgattactatagtcttatcgcttggaagaccattgccatatgtgtttttgggacggaggtcatttcggcaaagtttgtcatgaagacagatgatgacgcatttgttcgagtggatgaaatcttgtcttctatgcagaggattaatgtgactcgcggattgctctatggtcttatcaactcagattcccatcctcataggagcccagacagcaagtggtttatcagtccagaggaatggcctgaagaaacttaccctccttgggcacatggaccgggttatgttgtgtccagtgatatagcaaaaacaatcagctcaaaacagagaaaaggccgcctaaagatgtttaagctggaagatgttgctatgggcatctggatttcagaaataaagaagaaagggttggaagtgaagtatgaaaaagaagagaggatttttaatgaaggttgccgagatggttatgttattgcacattaccaaggccctagagagatgctctgtctttggcaaaagattgaagagaaaaaacgggctttatgttgtggcgagtaa

SEQ ID NO: 45 - protein, 548 aa, Nicotiana benthamiana; identical to SEQ ID NO: 29

SEQ ID NO: 46 - DNA, 1647 bp, Nicotiana benthamiana
tcagctcaggagcaacaatctttgtctacgtggtatcagttgaaacgcttagttactcatgatcgagtcttacctaatgctatagaggcagttaaggaagccagtatcgcatggaacaacctcatgagtgcagtagagagggaaaaacttgatgcaaatgatacttcagttaagacagggaaacagaaacaatgccctcattttctcagcaaaaccaatgctacagaaattgatgccagtggctttaagttgcgatttccttgtggtctgactcagggttcttccatcacaataattggcattccaaatggtcttcttgggaattttcggatagatttgactggtgaaccacttccaggtgaaccagatcctcctgttattctgcactacaatgttagactccatggtgataaaataactgaggatcccgtaattgtacaaaacacctggacaattgcacatgactggggtgaagaggagcgctgtcccttaccatcggatgaaaagagtaagaaagtggatgaattggaacaatgcaatgagatggtgggaaatatcatggctactcgacatgtcattgcaacaaataaatctagcctggctcaggacaggtctaaatcaagaaagtattttcctttcaagcaaggatatctctctgtcgccactctgagagtaggatctgaaggaattcagatgacagttgatggaaaacacataacatctttttctttccgtgaaactttggaaccgtggcttgtaagtgaagtgaggatatccggagacataaaattaatttctgttgtcgcaagtggtttgccaacatctgaggattcagagcatataagtgatttggaagctctaaaagcagctcctcttcctcctcggaaaagactagatctctttgttggtgtattttctaccgcaaataattttaagcgcagaatggctgtccgtagaacttggatgcaatatgatgcagtgcgatctggacaagttgcggtgcggttttttgtcggcttgcataaaaaccaaatggtgaatgaagagctctggaatgaggctaggacatatatggacatccagttgatgccttttgttgattactatagtcttatcgcttggaagaccattgccatatgtgtttttgggaccgaggtcgtttcggcaaagtttgtcatgaagacagatgatgacgcatttgttcgagtggatgaaatcttgtcttctatgcagaggattaacgtgactcgcggattgctctatggtcttatcaactcagattcccatcctcataggagtccagacagcaagtggtttatcagtccagaggaatggcctgaagaaacttaccctccttgggcacatggaccgggttatgttgtgtccagtgatatagcaaaacaatcagctcaaaacagaaaaaaaggccgcctaaagatgtttaagctggaagatgttgctatgggcatctggatttcagaaatgaataagaaagggttggaagtgaagtatgaaaaggaagagaggatttttaatgaaggttgccgagatggttatgttattgcacattaccaaggccctagagagatgctgtgtctttggcaaaagattgaagagaaaaaacgggctttatgttggggcgagtaa

SEQ ID NO: 47 - protein, 284 aa, Malus domestica
GGLKLISVLASGLPTSEDSEHIINLQELKSAPLSPHKQMDLFIGVFSTANNFKRRMAVRRTWMQYAAVRSGAVSVRFFVGLHKNQIVNEELWKEAQTYGDIQLMPFVDYYGLITWKTLAICIFGTEVVSAKFVMKTDDDAFVRVDEVLASLHRIKVNRGLLYGLINSDSRPHRSPDSKWYISNEEWPEATYPPWAHGPGYVVSNDIAKAVSQRYKKGRLKMFKLEDVAMGIWIADMKKEGLNVRYEKEEKVYNEGCKDGYVVAHYQGPREMLCLRQKIPKGQVA

SEQ ID NO: 48 - DNA, 855 bp, Malus domestica
ggaggcctgaaattaatttctgtcctagccagcggtttacccacgtcagaggattcagagcacataattaatttacaagaactcaaatctgctcctctctcgcctcacaaacaaatggatctctttattggtgttttctctaccgcaaacaattttaaacgacggatggctgttcgaagaacatggatgcagtatgctgctgtacgatcaggggcagtttcagtgcgcttttttgttggtttgcataagaaccaaatagtgaatgaggaactttggaaagaggcacaaacctatggagacatacagctgatgccttttgttgattactatggcctcattacctggaaaactctggccatctgcatctttgggacggaggttgtttcagcgaagttcgttatgaagacggatgacgatgcatttgttcgtgtggatgaagtgctagcttccttacacaggatcaaagtgaatcgtggcttgctctatggcctcattaactcagattctcgacctcatcgaagtccagatagcaagtggtatatcagcaatgaggaatggcctgaagcaacttacccaccttgggcgcacggtcctggttatgtggtatcgaatgacatagcaaaggcagtttcccagcgatataaaaagggcagattaaagatgtttaagctggaagacgtggcgatgggtatctggatcgcggatatgaaaaaggagggtctaaacgtacggtatgagaaggaagagaaggtctacaacgaaggatgcaaggacgggtatgtcgttgcacattaccaaggtcccagggagatgctgtgtctccggcagaaaattcctaaagggcaagttgcttga

SEQ ID NO: 49 - protein, 265 aa, Gossypium hirsutum
DHTVDIEALLSVPLSLQRPVDLFIGVFSTANNFKRRMAVRRTWMQYPEVRSGTVVVRFFVGLHKSHIVNDELWNEARTYGDIQLMPFVDYYSLITWKTLAICTFGTEVVSAKFVMKTDDDAFVRLDEVMASLSRINVTHGLLYGLINSDSQPHRSTDSKWFISPEEWSEEKYPPWAHGPGYVVSHDIAKAVYKRFNEVRLKMFKLEDVAMGIWIADMQKDGLEVHYEKEERIFNEGCKDGYVIAHYQGPREMMCLWQKLPGDQKS

SEQ ID NO: 50 - DNA, 798 bp, Gossypium hirsutum
gaccatacggttgatatagaagcactcctttcagttcctctttctcttcaaagaccagtggatctctttattggtgttttctctactgccaataattttaaaagaagaatggctgtgagaagaacatggatgcagtatcctgaagtccgatctgggacagttgtagtgcgcttttttgttggtttgcataaaagccatatagtgaatgatgaactctggaatgaagcacggacatatggagacatacagctgatgccttttgttgattactacagcctgatcacctggaaaaccttggctatttgcacctttgggacagaagttgtttctgcaaagtttgtcatgaagacagatgatgatgcgtttgttcgtttggatgaagtcatggcttcattaagcaggataaatgtaactcatggattgctttatggactcattaactcagattcccaacctcatcgcagtacagatagcaagtggtttattagccctgaggaatggtctgaagagaagtacccaccttgggcgcatggtcctggttacgtggtgtcccatgacatagcaaaggctgtttacaagaggttcaatgaagtgcgcttaaagatgtttaaactcgaagatgtggcaatgggcatttggatcgcggatatgcaaaaagatggtttagaagtccattatgagaaggaagagaggatctttaacgagggttgtaaggatggttatgttattgctcactatcaaggtccaagagagatgatgtgtttgtggcagaaactcccaggagaccaaaagagctag

SEQ ID NO: 51 - protein, 186 aa, Phaseolus vulgaris
PFVDYYSLITWKTLAICLFGTQVSAKFVMKTDDDAFVRVDEVLGSLHRINVDHGLLYGLINSDSRPHRNTDSKWYISPEEWSEGTYPPWAHGPGYVVSHDIARTVAKKFRENHLKMFKLEDVAMGIWIADMKKEGVDVRCENEDRVYPEGCKDGYVVAHYQGPREMLCLWQKLEEGKGAKCCGDRK

SEQ ID NO: 52 - DNA, 560 bp, Phaseolus vulgaris
ccatttgttgactactacagcctcatcacgtggaaaactttggcaatctgcctttttgggacacaagtttcggcaaagtttgtcatgaagacggatgatgatgcgtttgtcagggtagatgaagttctaggctctttgcacaggatcaatgttgatcatgggttactgtatggactcatcaattcggattccagacctcatcgaaatacagatagcaaatggtacatcagcccagaggaatggagcgaaggaacttatcctccttgggcacatggtcccggctatgtggtctcacacgatatagcaagaacggttgccaagaaattcagagagaaccatttgaagatgtttaaattagaagacgttgcaatgggaatttggattgcagacatgaagaaggaaggtgtagatgttcggtgtgagaatgaggacagagtgtatcctgagggttgtaaggatggttatgtggttgcccattaccagggccctagggagatgctctgtttgtggcagaaacttgaagaaggaaaaggtgcaaaatgctgtggtgataggaaatg

SEQ ID NO: 53 - protein, 235 aa, Euphorbia esula
TIDGKHITSFAYRETLEPWLVSEVRISGDMNLMSIVASGLPTSEDSEHAIDLELLKSVPLPSKKTPPPHLFIGVFSTANNFKRRMAVRRTWMQYPHVRSGTVAVRFFVGLHKNQLVNKELWNEASTYGDIQLMPFVDYYNLITWKTLAICIFGTEVASAKYVMKTDDDAFVRVDQVLASLKRIKVNRGLLYGLINSDSQPHRSTDSKWYISLEEWPEEKYPPWAHGPGYVVSQDI

SEQ ID NO: 54 - DNA, 707 bp, Euphorbia esula
accattgatggaaaacacatcacatcttttgcgtatcgcgagacattggagccatggctggttagcgaagttagaatatctggagacatgaatttaatgtctatagtggcaagtggtctacctacatcagaggattcagaacatgcaattgatctagagctactcaaatcggtccctctcccctcgaagaaaacaccaccacctcatctctttattggagtattctccacagccaacaattttaaacgtaggatggctgttcgaagaacttggatgcagtatcctcatgttcgctctgggactgttgctgtgcgcttttttgttgggttgcacaaaaatcaattagttaacaaggaactttggaatgaagcatcaacatatggagacatacagcttatgccttttgtggactattataacctcatcacctggaagactttagctatctgcatctttgggactgaggttgcatcagcaaaatatgtcatgaagacagatgatgatgcatttgttcgagtggatcaagttttagcttctttaaaacggatcaaagtgaatcgcggcctgctttatggactcataaattcagattcccagcctcataggagtactgatagcaaatggtatattagccttgaggaatggcctgaagagaaatatccgccatgggcacatggtcctggttatgtggtgtctcaagacatagc

SEQ ID NO: 55 - protein, 160 aa, Lactuca sativa
MKKWYIYVLSASLLTLTILGYLIIQNPIREAYISSPPYFNTTNPLEWINSSSPPVQNQTPNPQIVSTDEIVSTLFIHRNISFNEQQPLHTWNHLRHLISHDRVLPNTVEAIKDGGIAWNNLMTTVEDDKRDMNRSSGGKKKEKQCPHFLNKMNATELNDT

SEQ ID NO: 56 - DNA, 480 bp, Lactuca sativa
atgaagaaatggtatatctatgtcctatctgcatctctattaacattaacaatactcgggtacttaatcattcaaaatccaataagagaagcttacatttcatcacctccatacttcaacacaacaaaccctcttgaatggataaattccagttccccccctgttcaaaaccaaacccccaaccctcaaattgtctccactgatgaaatagtctccaccctctttatccacagaaacatctcattcaatgaacaacaaccattacacacatggaaccatttaagacacttaataagccatgatcgtgttcttcctaacacagttgaagcaatcaaagatggtggaatcgcatggaataacctcatgactacagttgaagatgacaaacgtgacatgaatagaagctcaggggggaaaaagaaagagaaacaatgtccccattttcttaacaagatgaatgccacagagcttaatgatact

SEQ ID NO: 57 - protein, 211 aa, Solanum lycopersicum
TMLCVFHETLEPWLISEVRISGDIKLISVVASGLPTSEDSEHISDLEALKAAPLPPRKRLDLFIGVFSTANNFKRRMAVRRTWMQYDAVRSGQVAVRFFVGLHKNQMVNEELWNEARTYRDIQLMPFVDYYSLITWKTIAICVFGTEVVSAKFVMKTDDDAFVRVDEILSSMERVNTTRGLLYGLINADSQPYRSPDSQWFISPEEWSEET

SEQ ID NO: 58 - DNA, 635 bp, Solanum lycopersicum
acgatgttatgtgtcttccatgaaactttggaaccatggctcataagtgaagtgaggatatcgggagacataaaattaatttctgttgtcgcaagtggtttaccaacatcagaggattcagagcatataagcgacttggaagccttaaaagcagctcctcttcctccacgaaaaagactagatctctttattggtgtattttctacagcaaataattttaagcgtagaatggctgtccgtagaacttggatgcaatatgatgcagtgcggtctggacaagttgcggtgcggttttttgttggcttgcataaaaaccaaatggtgaatgaagagctctggaatgaggctcggacatacagagacatccagctgatgccttttgttgattactacagtcttatcacttggaaaaccattgccatctgtgtttttgggacggaggttgtttctgcaaagtttgtcatgaagacagatgatgatgcatttgttcgagtggatgaaatcttatcttctatggagagggttaacacgactcgtggattgttatatggacttattaacgcagattcccaaccttacaggagtccagacagccagtggtttatcagtccagaggaatggtctgaagaaactta

SEQ ID NO: 59 - protein, 192 aa, Vitis vinifera
GAVAVRFFVGLHKNQMVNEELWKEVQTYGDIQLMPFVDYYSLITWKTIAICIFGTEAVSAKYVMKTDDDSFVRVDEVLASLKKTKVTHGLLYGLINSDARPHRSSDSKWYISPEEWAGETYPPWAHGPGYVVSNDIAKTVYKRHKEGHLKMFKLEDVAMGIWIAEMKKGGMEVSYIKEERVYNEGCNDGYVV

SEQ ID NO: 60 - DNA, 577 bp, Vitis vinifera
ggggcagttgccgtgcgcttttttgttggtctgcataaaaatcagatggtgaatgaggaactctggaaggaggtgcagacatatggagacattcagctgatgccttttgttgactattacagtcttataacctggaagaccatagcaatttgcatctttgggactgaggctgtttcggccaagtatgtgatgaaaacagatgatgattcatttgttcgtgtggatgaagtgcttgcttctttaaagaagaccaaagtgactcatgggttgctgtatggtcttattaactctgatgctcgacctcatcgtagttctgacagtaaatggtacatcagccctgaggaatgggcaggagaaacttatccaccttgggcacatggtcctggttacgtggtgtcaaatgacatagcaaaaacagtgtacaagagacacaaggaaggtcatttaaagatgttcaagctggaagatgtagcaatggggatctggatagcagagatgaagaaggggggaatggaggttagttacataaaagaagagagggtctataatgaggggtgcaacgatgggtacgttgttg

SEQ ID NO: 61 - protein, 231 aa, Triticum aestivum
  UNSURE (212)..(212)
AEPKKYYPFKQGNLAIAILRVGAEGIHMTVDGKHVTSFAFREDLEPGFVGEVRIEGDIKLLSVLASGLPTTEDFEHVTDLEILKAPPVPKDKSIDLFIGIFSTANNFKRRMAVRRTWMQYDAVRSGKVAVRFFVGLHKNDVVNKELWNEARTYGDIQLMPFVDYYSLILWKTIAICIYGTNVLSAKYVMKTDDDAFVRVDEILSSLHQVNIXHGLLYGRVNSDSQPHRDPY

SEQ ID NO: 62 - DNA, 693 bp, Triticum aestivum
  misc_feature (635)..(635): n is a, c, g, or t
gcagaacctaaaaaatattatcccttcaaacaaggaaatcttgctatagcaattcttcgtgttggagcagaggggatccatatgactgtagatgggaaacatgtcacttcctttgcatttcgagaggatttggagcctgggtttgttggtgaagtaaggattgaaggagatattaaattgctgtctgtgctagcaagtggcttgcctacaacagaggactttgagcatgtcactgacttggaaatattgaaggctccacctgtccctaaggataaatccattgatctttttattgggatattctctacagcaaataattttaaacgcagaatggcggttcgaagaacctggatgcagtacgatgctgtccgctcaggaaaagtcgctgttcggttctttgttggcctgcataaaaatgatgtggtgaacaaggagctctggaacgaagcacggacatatggagacatccaattgatgccatttgttgattattacagcttgattctttggaagaccattgccatttgcatatatggaacaaatgtactttcagccaagtatgttatgaaaaccgacgatgacgctttcgttcgtgtagatgagatactttcgtccctacatcaagtaaatatcanccatggactgctgtatggtcgcgtcaattctgattctcagcctcaccgagatccgtat

SEQ ID NO: 63 - protein, 193 aa, Solanum tuberosum
NNQVIPAEIVVSDLFIKRNLSAQEQQSLSTWYQLKRLITHDLVLPNAIEAVKEATVAWNNLMSAVEMDKLDTNDSSIKTGKQKQCPHFLSKTNATELDASGFKLRLPCGLTQGSSITIIGIPNGLLGNFRIDLTGEPLPGEPDPPVILHYNVRLHGDKITEDPVIVQNTWTIAHDWGEKERCPLPSDEKSKKV

SEQ ID NO: 64 - DNA, 580 bp, Solanum tuberosum
aataatcaagtaatacctgctgaaattgttgtctctgatctctttatcaagaggaacctgtcagctcaggagcaacaatctttgtctacgtggtatcaattgaaaaggttaatcactcatgatctagttttacctaatgctatagaggcggttaaggaagccactgttgcatggaacaacctcatgagtgcagtagagatggacaaactcgatacaaatgatagttcaattaagacagggaaacagaaacaatgtcctcattttcttagcaaaaccaatgctacagaacttgatgctagcggctttaagttgcggcttccttgtggtctgactcagggttcttccatcacaataattggcattccaaatggtcttcttgggaattttcggatagatttgactggtgaaccactaccaggtgaaccagaccctcctgttattctgcactacaatgttaggctccatggtgataaaataaccgaggatcctgtaatcgtacaaaacacctggactattgcacatgactggggtgaaaaggagcgctgtccattaccatccgatgaaaagagtaagaaagtgg

SEQ ID NO: 65 - protein, 137 aa, Medicago sativa
TLEPWLVSEVRIAGDLKLLSILASGLPTSEESEHVVDLEALKAPPLSPLRPLDLVIGVFSTANNFKRRMAVRRTWMQYDDVRSGRVAVRFFVGLHKSPIVNLELWNEARTYGDVQLMPFVDYYSLISWKTLAICIFG

SEQ ID NO: 66 - DNA, 411 bp, Medicago sativa
acactggagccatggcttgttagtgaagtacggattgcaggtgatttgaagttactatccattctcgctagcggtttacccacatcagaagaatcagagcatgttgttgatctagaggcactgaaggcacctcccctttcaccattaaggccattagatcttgtcattggcgttttctccacagcaaacaattttaaacgacggatggctgtgagaagaacttggatgcagtatgatgatgtaagatctggaagggttgcagtacgcttttttgttggccttcacaagagtcctattgttaacttggaactatggaacgaggctcggacttacggtgacgttcagctaatgccctttgttgattactacagtctcatcagttggaaaacactagcaatctgcatcttcggg

(19) United States
(12) Patent Application Publication
(10) Pub. No.: US 2009/0328251 A1
(43) Pub. Date: Dec. 31, 2009

(54) Galactosyltransferase

(75) Inventors: Richard Strasser, Vienna (AT); Herta Steinkellner, Vienna (AT); Lukas Mach, Langenzersdorf (AT); Josef Glossl, Vienna (AT); Friedrich Altmann, Vienna (AT); Jayakumar Singh Bondili, Vijayawada (IN)

Correspondence Address: FULBRIGHT & JAWORSKI L.L.P., 600 CONGRESS AVE., SUITE 2400, AUSTIN, TX, 78701, US

(73) Assignee: UNIVERSITAT FUR BODENKULTUR WIEN, Vienna (AT)

(21) Appl. No.: 12/443,384

(22) PCT Filed: Sep. 28, 2007
(86) PCT No.: PCT/EP07/08469

§ 371 Date: Mar. 27, 2009

(30) Foreign Application Priority Data
Sep. 29, 2006 (EP) 06450140.6

(51) Int. Cl. C12N 15/82; C12P 21/02; C12N 15/11; C12N 15/00; C07H 21/02; C12N 5/04; A01H 5/00

(52) U.S. Cl. 800/278; 435/69.1; 536/23.2; 435/320.1; 536/24.5; 435/419; 800/298

(57) ABSTRACT

The present invention provides a method of expressing a plant Lewis-type β1,3-galactosyltransferase in an organism comprising the step of providing the organism with a nucleic acid molecule comprising a sequence A which is defined as being selected from: a) a sequence according to SEQ ID NO: 1 with an open reading frame from base pair 1-1932, j) a sequence which is at least 50% identical with SEQ ID NO: 1, k) a sequence which hybridizes with SEQ ID NO: 1 under stringent conditions, or l) a sequence which has degenerated to SEQ ID NO: 1 due to the genetic code, wherein the sequences a) to d) encode a plant protein having Lewis-type β1,3-galactosyltransferase activity, or m) a sequence which is complementary to one of the sequences a) to d), and expressing a protein encoded by sequence A or if the organism comprises a sequence A in its wildtype form overexpressing a protein encoded by sequence A.

The present invention relates to the field of protein glycosylation engineering in plants, specifically to the production of glycoproteins in plants or plant cells.

Recombinant glycoproteins, like monoclonal antibodies show great promise as therapeutics and the demand for their production is steadily increasing. Hence, apart from the traditional ways of manufacturing recombinant proteins in living mammalian cells, mainly in Chinese hamster ovary cells, currently alternative expression systems, such as yeast, fungi, plants, insects and transgenic animals are being intensively investigated.

Production of recombinant glycoproteins in plants has the advantage of low cost, short generation times and easy scale-up. However, the N-glycan structures of plant-produced glycoproteins differ substantially from those found in animals. These differences in N-glycosylation can have important consequences on product safety and quality. It is well documented that β1,2-xylose and core α1,3-fucose containing N-linked glycans are immunogenic and potentially allergenic. The presence of these potentially immunogenic and/or allergenic plant-specific N-linked glycans therefore hinders the use of plants as production system for therapeutic glycoproteins. Furthermore, differences in N-glycosylation affect also the function and quality of the glycoprotein, for example due to reduced half-life of the drug, which results in the need to use higher doses. Achieving a proper glycosylation pattern as present on the natural human forms is therefore a major challenge for the production of therapeutic glycoproteins in plants.

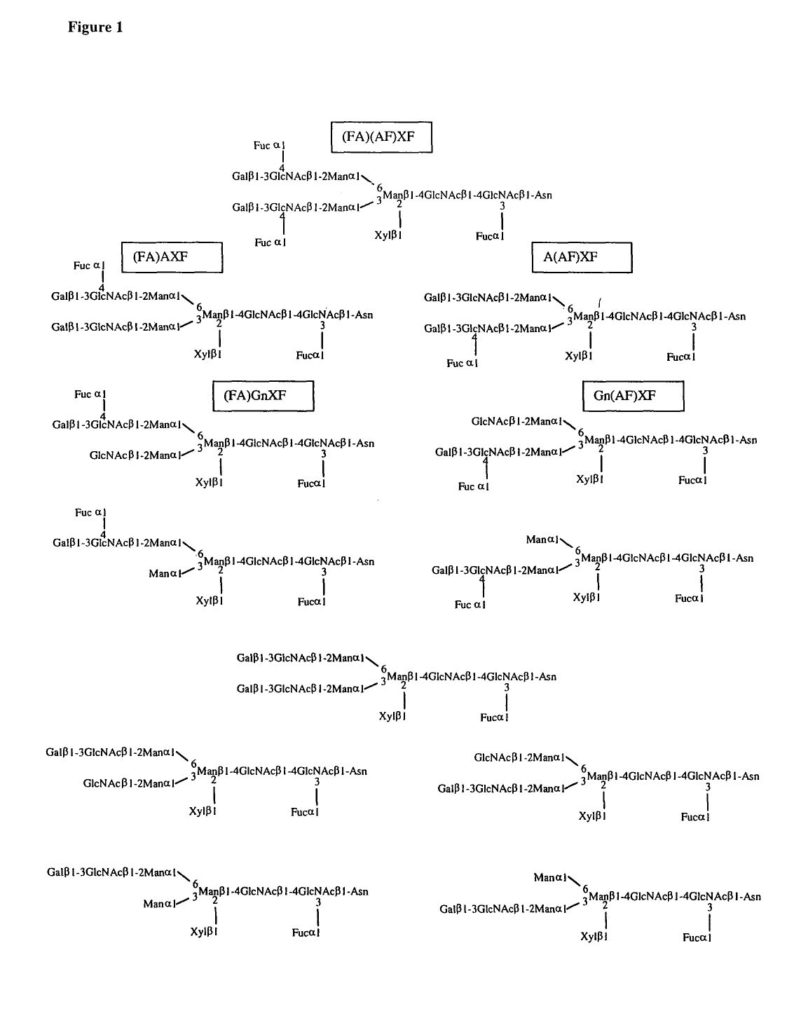

N-glycans from plants are typically much smaller and fewer glycoforms are detected compared to their mammalian counterparts. Plant glycoproteins contain two major types of oligosaccharides: paucimannosidic and complex N-glycans. Complex plant N-glycans carry β1,2-xylose and core α1,3-fucose, which are absent in mammalian glycoproteins, as well as one or two terminal N-acetylglucosamine (GlcNAc) residues attached to the pentasaccharide (Man₃GlcNAc₂) core structure. Plant glycoproteins differ from mammalian ones as they are devoid of β1,4-linked galactose and sialic acid. The only known plant N-glycan chain elongation is the attachment of a β1,3-galactose (Gal) and a α1,4-fucose (Fuc) residue, which form a trisaccharide (Galβ1-3(Fucα1-4)Glc-NAC-R) known as the Lewis-A epitope (FIG. 1).

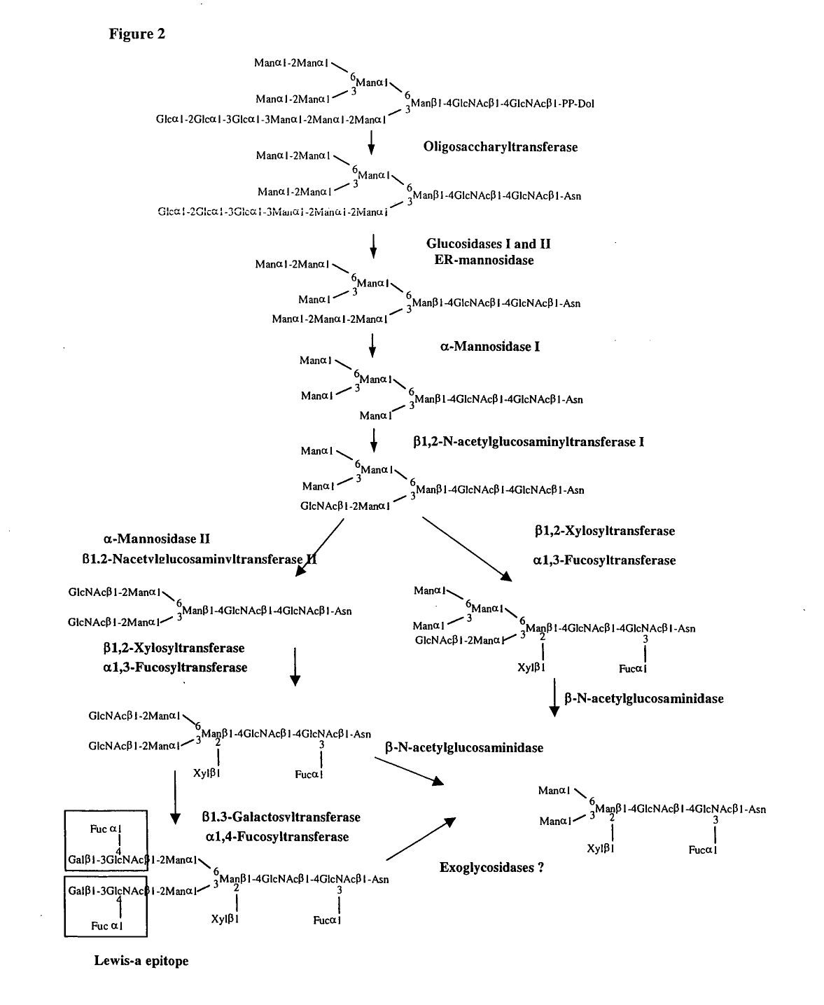

The N-glycosylation pathway in plants is well characterized (FIG. 2). The genes which are responsible for the addition of GlcNAc residues (β1,2-N-acetylglucosaminyltransferase I, β1,2-N-acetylglucosaminyltransferase II), processing (Golgi α-mannosidase II), attachment of β1,2-linked xylose (β1,2-xylosyltransferase) and core α1,3-linked fucose (core α1,3-fucoslytransferase) on complex N-glycans in plants have been cloned and characterized.

Lewis antigens, including type 1 (Le^{a}, Le^{b}) and type 2 chain (Le^{x}, Le^{y}) carbohydrates, are fucosylated oligosaccharide epitopes identified on the surface of certain eukaryotic cells. The Lewis-A antigen is structurally related to determinants of the human ABH blood group system. It is rare as such in healthy adults but as sialyl-Lewis-A it is notoriously found in malignant tissues but usually not attached to mammalian protein N-linked glycans.

In plants the Lewis-A epitope is present in many species and it is detected on plant produced recombinant glycoproteins.

Hence, the presence of the Lewis-A carbohydrate structure represents a significant barrier for the production of recombinant glycoproteins in plants, since this epitope is usually not present on natural human or animal glycoproteins intended for therapeutic use (e.g. on erythropoietin). As a consequence there is a need for technologies to eliminate Lewis-A epitopes from plants and also from plant made recombinant glycoproteins. However, up to now it has not been possible to modulate this in many cases unwanted glycan epitope, simply due to the fact, that the gene/enzyme that initiates the biosynthesis of the Lewis-A epitope in plants was not known.

Two enzymes are required for the synthesis of the plant Lewis-A epitope linked to complex N-glycans. First, the plant Lewis-type β1,3-galactosyltransferase transfers a galactose to a GlcNAc residue with a β1,3-linkage, resulting in the synthesis of a type 1 chain (Galβ1-3GlcNAc), and then the plant Lewis-type α1,4-fucosyltransferase transfers a fucose to the GlcNAc of the type 1 chain with an α1,4-linkage to complete the synthesis of the Lewis-A structure (Galβ1-3(Fucα1-4)GlcNAc). The Lewis-type α1,4-fucosyltransferase has been cloned from several plant species and the corresponding enzyme activity has been characterized. The plant Lewis-type α1,4-fucosyltransferase has a strict acceptor substrate specificity for type 1 chain-based glycan structures whereas type 2 chain-based oligosaccharides (Galβ1-4GlcNAc), which are typical for mammalian glycoproteins, are not utilized. A Lewis-type β1,3-galactosyltransferase has neither been cloned from plants nor has the corresponding activity been described in plants.

What is needed, therefore, is a method to eliminate the Lewis type β1,3-galactosyltransferase from plants and a method to eliminate the Lewis-A epitope from plant-produced recombinant glycoproteins. This will enable the production of recombinant glycoproteins in plants with authentic N-linked glycan structures, devoid of Lewis-A epitopes.

Another object of the present invention is to provide a plant protein having Lewis-type β1,3-galactosyltransferase activity, and a DNA encoding the protein.

In a first embodiment the invention provides a method of expressing a plant Lewis-type β1,3-galactosyltransferase in an organism comprising the step of providing the organism with a nucleic acid molecule comprising a sequence A which is defined as being selected from
a) a sequence according to SEQ ID NO: 1 with an open reading frame from base pair 1-1932,
b) a sequence which is at least 50% identical with SEQ ID NO: 1,
c) a sequence which hybridizes with SEQ ID NO: 1 under stringent conditions, or
d) a sequence which has degenerated to SEQ ID NO: 1 due to the genetic code,
   wherein the sequences a) to d) encode a plant protein having Lewis-type β1,3-galactosyltransferase activity, or
e) a sequence which is complementary to one of the sequences a) to d),
   and expressing a protein encoded by sequence A or if the organism comprises a sequence A in its wildtype form overexpressing a protein encoded by sequence A. Preferably the nucleic acid molecule is a DNA molecule or a RNA or protein nucleic acid.

The present invention provides for the first time a sequence associated with the formation of the Lewis-A epitope in plants. This sequence can now for the first time be used for (over)expressing in plants for the production of glycoproteins comprising the Lewis-A epitope e.g. as a vaccine for immunotherapies. This is of particular interest since several types of cancer express the Lewis-A epitope in humans and mammals.

Although the sequence of SEQ ID NO: 1 was previously described (NCBI database AY133724) no explicit function especially the Lewis-type β1,3-galactosyltransferase activity was known. This knowledge however is of critical importance for any use therefore, including the design of knock-out constructs for the suppression of the Lewis-type β1,3-galactosyltransferase activity.

The activity of the plant Lewis-type β1,3-galactosyltransferase is detected by a method and measured, the galactosyltransferase being added to a reaction mixture containing UDP-galactose and a labeled acceptor (e.g. a glycopeptide or labeled oligosaccharide). After the reaction time, the content of bound galactose is measured. The activity of the galactosyltransferase in this case is seen as positive if the activity measurement is higher by at least 10 to 20%, in particular at least 30 to 50%, than the activity measurement of the negative control. The structure of the oligosaccharide may additionally be verified by means of HPLC. Such protocols are prior art (Staudacher et al., 1998, Anal. Biochem. 246: 96-101; Staudacher et al., 1991, Eur. J. Biochem. 199: 745-751). Whether the galactose is bound or not to the acceptor substrate can furthermore be determined by measuring the mass of the product by means of mass spectrometry (e.g. Strasser et al., 2000, FEBS Lett. 472: 105-108).

Lewis-A epitope can be detected on glycoproteins from plants by immunoblots using JIM84 antibody (Horsley et al., 1993, Journal of Exp. Bot. 44, Supplement: 223-229), which is specific for the plant Lewis-A epitope (Fitchette et al., 1999, Plant Physiol. 121: 333-343) and subsequently by total N-glycan analysis using mass spectrometry.

Preferably the protein encoded by the nucleic acid molecule having Lewis-type β1,3-galactosyltransferase activity has a sequence of SEQ ID NO: 2 or is at least 50% identical with SEQ ID NO: 2. In particular preferred the protein comprises the galactosyltransferase domain according to SEQ ID NO: 4, or a galactosyltransferase domain which is at least 50% identical with SEQ ID NO: 4. Thus the present invention provides for a group of homologuous enzymes with e.g. up to 5, 10, 20, etc. mutations which retain the Lewis-type β1,3-galactosyltransferase activity. In particular embodiments the sequence of the peptide is at least 60%, at least 70%, at least 75%, at least 80%, at least 90%, at least 95%, or at least 99% identical with SEQ ID NO: 2 or with the domain of SEQ ID NO: 4. Also provided in the method wherein the expression or overexpression is facilitated by transfecting the organism with a nucleic acid comprising a sequence A or by upregulation of expression of an endogenous sequence A.

Upregulation can be achieved by any means known in the art, especially the use of promoters or promoter-enhancer constructs. The constructs regulate the expression of sequence A on the nucleic acid molecule. Preferred examples for Lewis-type β1,3-galactosyltransferase expression enhancing compounds are substances which up-regulate Lewis-type β1,3-galactosyltransferase, e.g. toxins if expression of the transferase is controlled by toxin-inducible promoters on the nucleic acid. In principle, any cell which can be genetically manipulated so that it expresses a recombinant (i.e. a gene which has been artificially introduced into this cell or its precursor and which naturally is not present at this site of the genome of the cell) plant Lewis-type β1,3-galactosyltransferase gene can be used for this purpose. Preferably the promoter regulates increased expression when compared to the wildtype organism or is externally inducible to an increased expression when compared to the wildtype organism, preferably plant or plant cell. Upregulation can be achieved by transfecting the organism with a construct with sequence A or by stimulating endogenous expression of the Lewis-type β1,3-galactosyltransferase.

To facilitate understanding of the invention, a number of terms as used in this specification are defined below:

The term “glycosyltransferase” refers to any enzyme that transfers monosaccharide-residues from nucleotide sugar donor substrates to a carbohydrate structure.

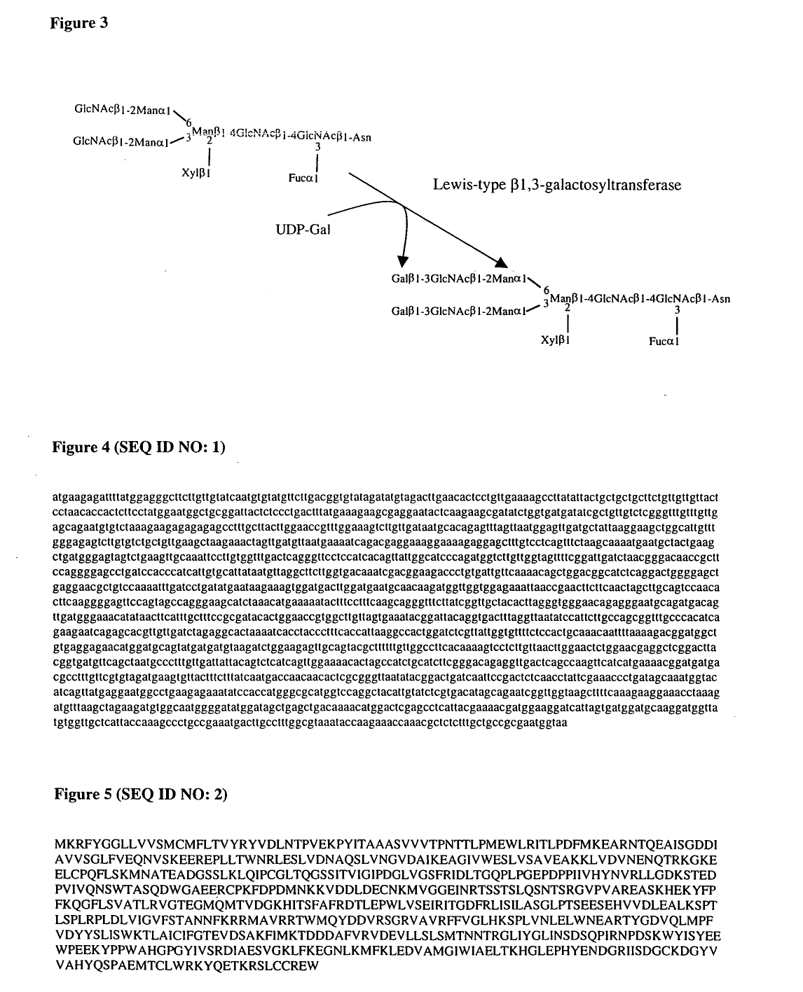

The term “Lewis-type β1,3-galactosyltransferase” (“Lewis-type β1,3-GalT”) refers to any glycosyltransferase that catalyses the first step in the formation of the Lewis-A epitope. The first step in the formation of the Lewis-A epitope is the transfer of a galactose residue in β1,3-linkage to a GlcNAc residue at the non-reducing end of a carbohydrate side-chain (see FIG. 3). This reaction creates the type 1 chain structures (Galβ1-3GlcNAc), which can be further utilized by α1,4-fucosyltransferases. Typical plant N-glycans containing Lewis-A epitopes are illustrated in FIG. 1. The Lewis-type β1,3-galactosyltransferase transfers galactose from UDP-α-D-galactose to the β-linked acceptor sugar GlcNAc at the non-reducing terminus of a carbohydrate side-chain of a glycoprotein in plants.

The term “humanization” or “humanized N-glycan structures” refers to steps—inactivation of endogenous plant enzymes and introduction of mammalian enzymes, which lead to the formation of protein linked N-glycans in plants which are more similar or identical to the N-glycans of humans (or mammals) than N-glycans on glycoproteins from wildtype [“non-humanized”] plants.

The term “organism” as used herein refers to all organisms and in particular organisms containing glycoproteins with N-linked glycans.

The term “plant” as used herein refers to a plurality of plant cells which are largely differentiated into a structure that is present at any stage of a plant's development. Such structures include, but are not limited to, a fruit, shoot, stem, root, leaf, silique, seed, flower petal, or similar structure.

The term “plant tissue” includes differentiated and undifferentiated tissues of plants including, but not limited to, roots, shoots, leaves, pollen, seeds, tumor tissue and various types of cells in culture (e.g., single cells, protoplasts, embryos, callus and other types of cells). Plant tissue may be in planta, in organ culture, tissue culture, or cell culture. Similarly, “plant cells” may be cells in culture or may be part of a plant.

The term “transfection” into a cell or “transfected” in the context of nucleic acid (e.g. vectors) is intended to include what the art calls “transformation” or “transduction”. Transformation of a cell may be stable or transient. The present invention comprises introduction of vectors under conditions where, on the one hand, there is stable expression, and on the other hand, where there is only transient expression.

The term “transient transformation” or “transiently transformed”refers to the introduction of one or more transgenes into a cell in the absence of integration of the transgene into the host cell's genome. Transient transformation may be detected by, for example, enzyme-linked immunosorbent assay (ELISA) which detects the presence of a polypeptide encoded by one or more of the transgenes. Alternatively, transient transformation may be shown by detecting the activity of the protein (e.g. antigen binding of an antibody) encoded by the transgene (e.g. the anti-body gene). The term “transient transformant” refers to a cell which has transiently incorporated one or more transgenes. In contrast, the term “stable transformation” or “stably transformed” refers to the introduction and integration of one or more transgenes into the genome of a cell. Stable transformation of a cell may be detected by Southern blot hybridization of genomic DNA of the cell with nucleic acid sequences which are capable of binding to one or more of the transgenes. Alternatively, stable transformation of a cell may also be detected by the polymerase chain reaction (PCR) of genomic DNA of the cell to amplify transgene sequences. The term “stable transformant” refers to a cell which has stably integrated one or more trans-genes into the genomic DNA. Thus, a stable transformant is distinguished from a transient transformant in that, whereas genomic DNA from the stable transformant contains one or more trans-genes, genomic DNA from the transient transformant does not contain a transgene.

The term “sequence identity” refers to identity between two sequences, usually amino acid sequences, which can be determined by sequence alignment programs like BLAST, PSI-BLAST (Altschul et al., 1990, J. Mol. Biol. 215: 403-410; Altschul et al., 1997, Nucleic Acids Res. 25: 3389-3402; http://www.ncbi.nlm.nih.gov/blast/) or ClustalW (Chema et al., 2003, Nucleic Acids Res. 31: 3497-500; e.g. http://www.ebi.ac.uk/clustalw/). These algorithms calculate the best match for the selected sequences, and line them up so that the identities, similarities and differences can be seen.

The term “stringent condition” relates to a condition that allows the specific pairing of two DNA molecules that is influenced by selection of the temperature and ionic strength of the sample. By stringent conditions, according to the invention conditions are understood which allow for a stringent binding with no significant background. For instance, the DNA molecules are hybridized in 7% sodium dodecyl sulfate (SDS), 0.5M sodium phosphate, pH 7.2, 1 mM EDTA, 1% bovine serum albumine at 65° C., and washed with 40 mM sodium phosphate buffer, pH 7.2, 1 mM EDTA, 1% SDS at 65° C. Higher stringency is e.g. a hybridization in 5×SSC at 50° C. or 5×SSC at 65° C. (very high stringency). To identify genes that share >65% sequence identity a hybridization at low stringency can be performed according to Sambrook et al. 2001.

Hybridizations may be performed in a buffer containing 30% deionized formamide, 0.6M NaCl, 0.04 M sodium phosphate, pH 7.4, 2.5 mM EDTA, 1% SDS at 42° C. At the end of the hybridization washing is performed with 2×SSC/0.1% SDS at room temperature and in 2×SSC/0.1% SDS at 55° C.

The invention also discloses a nucleic acid molecule comprising a sequence according to SEQ ID NO: 1 with an open reading frame from base pair 1 to base pair 1932 or being at least 50% identical to the above sequence or hybridizing with the above indicated sequence under stringent conditions, or comprising a sequence which has degenerated to the above nucleic acid sequence due to the genetic code, the sequence coding for a plant protein, which has plant Lewis-type β1,3-galactosyltransferase activity or is complementary thereto. With the knowledge of its activity such a nucleic acid molecule derived from SEQ ID NO: 1 can be used especially for the inactivation or suppression of the plant Lewis-type β1,3-galactosyltransferase activity as well as for overexpression and production of the recombinant enzyme. Complementary sequences can be used either to express the protein or to inhibit expression via RNA binding. To express the enzyme or the RNA a promoter has to be placed at the right position and direction for expression from the (in vivo formed) double strand.

In further embodiments the present invention provides a plant protein having a plant Lewis-type β1,3-galactosyltransferase activity, a nucleic acid encoding the protein, a recombinant DNA containing the DNA, a transformant containing the recombinant DNA, a process for producing a protein having a plant Lewis-type β1,3-galactosyltransferase activity using the transformant and a method for producing a galactose-containing carbohydrate using the transformant. The present invention further relates to a method for inactivation or inhibition of the plant Lewis-type β1,3-galactosyltransferase activity and a method for elimination of the Lewis-A carbohydrate epitope from plant glycoptroteins and plant-produced glycoproteins that are particularly useful as therapeutic agents.

Alternative strategies, which aim at the inactivation of glycosylation steps, that take place earlier and are a prerequisite for β1,3-galactosyltransferase have the disadvantage that they affect not only the formation of the Lewis-A epitope but also change the overall glycosylation machinery of the host plant. For example the inactivation of N-acetylglucosaminyltransferase I leads to the production of only oligo-mannosidic N-linked glycan structures (von Schaewen et al., 1993, Plant Physiol. 102: 1109-1118; Strasser et al., 2005, Biochemical J. 387: 385-391). As a consequence recombinant glycoproteins produced in such plants contain oligo-mannosidic N-glycans and no complex-type N-glycans (Downing et al., 2006, Plant Biotechn. J., 4: 169-181). Such glycans cannot be further modified in vivo and represent no improvement towards the humanisation of the plant N-glycosylation pathway, since these oligomannosidic structures are not common on mammalian glycoproteins and thus represent “non-authentic” N-glycosylation. Other alternative strategies to circumvent the attachment of unwanted potentially immunogenic or allergenic sugar-residues to glycoproteins include the retention in the ER (e.g. by adding an ER retention signal; Gomord et al., 2005, Trends in Biotechnology, 23: 559-565) or Golgi bypassing by a direct transport of a recombinant protein from the ER to a protein storage vacuole (Wang et al., 2006, Trends in Biotechn., 24: 147-149). However, all these strategies prevent the formation of N-glycan processing and lead to the formation of oligo-mannosidic N-glycan structures (e.g. Man₇GlcNac₂), which are far from being a mammalian-like N-glycan pattern found on glycoproteins intended for therapeutic use. Besides being non-authentic these oligo-mannosidic structures affect also the quality of the product, e.g. reduced half-life due to rapid uptake by high-mannose receptors present on macrophages and endothelial cells.

For the specific suppression or inactivation of proteins it is best to carry this out at the level of transcription and translation, respectively. For this it is necessary to isolate and characterize the nucleotide sequence which codes for the active protein. It is an object of the present invention to clone and to sequence the whole gene, which codes for a plant Lewis-type β1,3-galactosyltransferase and to prepare vectors comprising this gene or an altered DNA or a DNA derived therefrom, to transfect plants as well as cells thereof with one of these vectors, to produce glycoproteins that do not contain the normally occurring β1,3-linked galactose attached to N-glycans, as well as to provide corresponding methods thereof.

Therefore, the present invention provides a method for elimination of the Lewis-A epitope from glycoproteins produced in plants. To block specifically the formation of the Lewis-A epitope linked to N-glycans without also interfering in other glycosylation steps, solely that enzyme would have to be inactivated which is directly responsible for this specific glycosylation, i.e. the Lewis-type β1,3-galactosyltransferase. This glycosyltransferase would have to be inactivated on purpose or suppressed so that human or other mammalian proteins which are produced in plants or in plant cells, respectively, would no longer contain this potentially immune-reaction-triggering epitope, as has been the case so far. This inactivation (or at least reduction of activity) of Lewis-type β1,3-GalT can be done in many ways on many levels by a skilled man in the art on the basis of the information contained herein. For example, expression (or transcription, translation, etc.) of the Lewis-type β1,3-GalT may be hindered or made impossible by recombinant DNA/RNA technology. On the other hand, the activity of the enzyme may be reduced or abolished by introduction of mutations in the coding sequence of the gene of this protein or by inactivating the enzyme by inhibitors (by specific antibodies of the Lewis-type β1,3-GalT, etc.) The inactivation of the Lewis-type β1,3-galactosyltransferase will also block the transfer of α1,4-linked fucose, since the type 1 chain structure is required as a substrate for the plant Lewis-type α1,4-fucosyltransferase.

Also provided is the use of a nucleic acid molecule comprising a sequence A as defined above for expressing a plant Lewis-type β1,3-galactosyltransferase.

In another embodiment the present invention provides for a nucleic acid molecule comprising a sequence derived from sequence A further comprising a disruption, deletion, insertion and/or substitution mutation, preferably in the galactosyltransferase domain as defined in claim 3, encoding an inactive Lewis-type β1,3-galactosyltransferase, preferably encoded by a sequence as defined in claims 1 to 3. The number of mutant nucleotides is variable and varies from a single one to several deleted, inserted or substituted nucleotides. It is also possible that the reading frame is shifted by the mutation. In such a “knockout gene” it is merely important that the expression of a β1,3-galactosyltransferase is disturbed, and the formation of an active, functional enzyme is prevented. In doing so, the site of the mutation is variable, as long as expression of an enzymatically active protein is prevented. Preferably, the mutation is in the catalytic region of the enzyme which is located in the C-terminal region. The method of inserting mutations in DNA sequences are well known to the skilled artisan, and therefore the various possibilities of mutageneses need not be discussed here in detail. Coincidental mutageneses as well as, in particular, directed mutageneses, e.g. the site-directed mutagenesis, oligonucleotide-controlled mutagenesis or mutagenesis by aid of restriction enzymes may be employed in this instance. Of course, all nucleic acid and protein molecules according to the present invention are referred to herein in isolated form (if not explicitly disclosed otherwise).

Preferably the nucleic acid molecule with the inactive enzyme hybridizes to SEQ ID NO: 1 under stringent conditions. A hybridization reaction in vivo can result in an disruption, deletion, insertion and/or substitution mutation of the endogenous Lewis-type β1,3-galactosyltransferase. Preferably the organism is a plant or plant cell, especially preferred Arabidopsis or tobacco.

Preferably the nucleic acid molecule comprising a disrupted Lewis-type β1,3-galactosyltransferase gene has a disruption by insertion of an exogenous sequence into said gene such that the disruption prevents expression of functional β1,3-galactosyltransferase, wherein the gene, prior to disruption, codes for a plant Lewis-type β1,3-galactosyltransferase with an amino acid sequence of SEQ ID NO: 2 or with at least 50% identity to SEQ ID NO: 2. The exogeneous sequence can be of any size, e.g. 1, 2, 3, up to 10, up to 50, up to 100, up to 200 nucleotides or also up to 5,000 nucleotides or up to 10,000 nucleotides in length, and can be inserted, for example at any position e.g. in the galactosyltransferase domain of SEQ ID NO: 1. Preferably the exogeneous sequence is an transformation marker and a successful transfection can thus be easily detected.

The invention further provides a nucleic acid molecule which codes for a ribozyme which comprises two sequence sections, each of which has a length of at least 10 to 15 base pairs each, which are complementary to sequence sections of an inventive DNA molecule as described above so that the ribozyme complexes and cleaves the mRNA which is transcribed from a natural plant Lewis-type β1,3-galactosyltransferase DNA molecule. The publication by John M. Burke “Clearing the way for ribozymes” (Nature Biotechnology 15: 414-415; 1997) relates to the general mode of function of ribozymes. The ribozyme will recognize the mRNA of the plant Lewis-type β1,3-galactosyltransferase by complementary base pairing with the mRNA. Subsequently, the ribozyme will cleave and destroy the RNA in a sequence-specific manner, before the enzyme is translated. After dissociation from the cleaved substrate, the ribozyme will repeatedly hybridize with RNA molecules and act as specific endonuclease. In general, ribozymes may specifically be produced for inactivation of a certain mRNA, even if not the entire DNA sequence which codes for the protein is known. Ribozymes are particularly efficient if the ribosomes move slowly along the mRNA. In that case it is easier for the ribozyme to find a ribosome-free site on the mRNA. For this reason, slow ribosome mutants are also suitable as a system for ribozymes (Burke, 1997, Nature Biotechnology; 15: 414-415).

One possible way is also to use a varied form of a ribozyme, i.e. a minizyme. Minizymes are efficient particularly for cleaving larger mRNA molecules. A minizyme is a hammer head ribozyme which has a short oligonucleotide linker instead of the stem/loop II. Dimer-minizymes are particularly efficient (Kuwabara et al., 1998, Nature Biotechnology, 16: 961-965).

If the vector comprising the nucleic acid molecule coding for a ribozyme is transfected, the active ribozyme will be expressed in the host cell. The ribozyme complexes the complementary mRNA sequence of the Lewis-type β1,3-galactosyltransferase at least at a certain site, cleaves this site, and in this manner it can inhibit the translation of the enzyme. In this host cell as well as in cell lines, or optionally, plant, respectively, derived therefrom, Lewis-type β1,3-galactosyltransferase will not be expressed.

A further aspect of the invention relates to a biologically functional vector which comprises one of the above indicated nucleic acid molecules, preferably DNA, as well as the method of using the vector to transfect an organism, preferably suitable to transfect a plant or a plant cell. For transfection into host cells, an independent vector capable of amplification is necessary, wherein, depending on the host cell, transfection mechanism, task and size of the nucleic acid molecule, a suitable vector can be used. Since a large number of different vectors is known, an enumeration thereof would go beyond the limits of the present application and therefore is done without here, particularly since the vectors are very well known to the skilled artisan (as regards the vectors as well as all the techniques and terms used in this specification which are known to the skilled artisan, cf. also Maniatis). Ideally, the vector has a small molecule mass and should comprise selectable genes so as to lead to an easily recognizable phenotype in a cell so thus enable an easy selection of vector-containing and vector-free host cells. To obtain a high yield of DNA and corresponding gene products, the vector should comprise a strong promoter, as well as an enhancer, gene amplification signals and regulator sequences. For an autonomous replication of the vector, furthermore, a replication origin is important. Polyadenylation sites are responsible for correct processing of the mRNA and splice signals for the RNA transcripts. If phages, viruses or virus particles are used as the vectors, packaging signals will control the packaging of the vector DNA. For instance, for transcription in plants, Ti plasmids are suitable.

If the above described inventive vector is inserted into a plant or into a plant cell, a post-transcriptional suppression of the gene expression of the endogenous Lewis-type β1,3-galactosyltransferase gene is attained by transcription of a transgene homologous thereto or of parts thereof, in sense orientation. For this sense technique, furthermore, reference is made to the publications by Baulcombe 1996, Plant. Mol. Biol., 9: 373-382, and Brigneti et al., 1998, EMBO J., 17: 6739-6746. This strategy of “gene silencing” is an effective way of suppressing the expression of the plant Lewis-type β1,3-galactosyltransferase gene (see also Waterhouse et al., 1998, Proc. Natl. Acad. Sci. USA, 95: 13959-13964).

Furthermore, the invention relates to a biologically functional vector comprising a DNA molecule according to one of the above described embodiments, being inversely orientated with respect to the promoter. If this vector is transfected in a host cell, an “antisense mRNA” will be read, which is complementary to the mRNA of the β1,3-galactosyltransferase and complexes the latter. This bond will either lead to the cleavage of the mRNA or hinder correct processing, transportation, stability or, by preventing ribosome annealing, it will hinder translation and thus the normal gene expression of the Lewis-type β1,3-galactosyltransferase. Although the entire sequence of the DNA molecule could be inserted into the vector, partial sequences thereof because of their smaller size may be advantageous for certain purposes. Different sequence regions of the DNA molecule may be inserted into the vector. One possibility consists, e.g., in inserting into the vector only that part which is responsible for ribosome annealing. Blocking in this region of the mRNA will suffice to stop the entire translation (e.g. microRNAs can suppress translation). A particularly high efficiency of the anti-sense molecules also results for the 5′- and 3′-untranslated regions of the gene.

For a particularly effective inhibition of the expression of an active Lewis-type β1,3-galactosyltransferase, a combination of the sense technique and the antisense technique is suitable (Waterhouse et al., 1998, Proc. Natl. Acad. Sci., USA, 95: 13959-13964). Especially the use of “double-stranded RNA” (dsRNA), e.g. generated from transgenes that produce dsRNA (inverted repeats constructs or hairpin constructs), is suitable for inhibition of the expression of an active Lewis-type β1,3-galactosyltransferase. The potent gene silencing process using dsRNA, known also as RNA interference (RNAi), mainly induces silencing through mRNA breakdown in plants. The design of such gene silencing constructs is well known to the artisan and described in several publications (e.g. Smith et al., 2000, Nature, 407: 319-320; Wesley et al., 2001, Plant J., 27: 581-590; Douchkov et al., 2005, Mol. Plant. Microbe Interact., 18: 755-761).

According to a specific embodiment of the invention the inhibition of the Lewis-type β1,3-galactosyltransferase is performed by double-stranded “small interfering RNAs” (siRNAs), which are e.g. delivered biolistically into plants cells (Vanitharani et al., 2003, Proc. Natl. Acad. Sci. USA., 100: 9632-9636). SiRNA molecules or microRNAs are provided which have a base sequence of 10 to 100 base length, preferably 15 to 40 bases, more preferred 18 to 30 bases or double-stranded hairpin-RNA comprising a base sequence of 10 to 1000, preferably 50 to 500, especially 100 to 300 bases of SEQ ID NO: 1 or being complementary to SEQ ID NO: 1. For this purposes also sequences not being 100% identical to SEQ ID NO: 1 also with certain base exchanges silencing is still enabled. For this purpose, accordingly homologous sequences of other organisms can be used. The use of siRNAs mainly leads to cleavage of the target mRNA but can also affect the chromatin structure of the target gene, resulting in transcriptional inhibition (Lippman and Martienssen, 2004, Nature 431: 364-370).

Preferably the inhibition of the Lewis-type β1,3-galactosyltransferase according to the present invention is performed by “microRNAs”, which are processed in the nucleus from a precursor, and either lead to microRNA-mediated cleavage of the target mRNA or to microRNA-mediated translation prevention (e.g. reviewed by Bartel, 2004, Cell 116: 281-297).

A further embodiment of the present invention relates to the inhibition of Lewis-type β1,3-GalT being performed by “artificial micro RNAs”. The design principle and use for artificial micro RNAs is described in detail by Schwab et al., 2006, Plant Cell 118: 1121-1133.

Preferably, the inhibition of the Lewis-type β1,3-GaltT by these molecules (sense, antisense, dsRNA, siRNAs, microRNAs, artificial microRNAs, etc.) is performed by vectors comprising the respective molecules, whereby the vectors will be used to trans-form plants or plant cells.

According to another preferred embodiment of the invention the inhibition of the Lewis-type β1,3-galactosyltransferase by these molecules (sense, antisense, dsRNA, siRNAs) is performed by virus comprising the respective molecules (virus induced gene silencing: e.g. Lu et al., 2003, Methods, 30: 296-303).

According to another aspect of the invention a method of reducing or preventing the expression of an endogenous plant Lewis-type β1,3-galactosyltransferase in an organism, preferably a plant or plant cell, is provided comprising the step of (at least transiently) transfecting the organism with a nucleic acid molecule or a vector or treating the organism with a RNA as disclosed herein. The inhibition of the Lewis-type β1,3-galactosyltransferase is e.g. performed by insertional mutagenesis, whereby a T-DNA from Agrobacteria can be inserted into the target gene (Krysan et al., 1999, Plant Cell, 11: 2283-2290) or a transposon element (e.g. Kumar et al., 2005, Plant J., 44: 879-892) can be utilized for insertion into the target gene. The method includes, but is not limited to insertion into an exon of the target gene, into an intron of the target gene or into regulatory sequence, like the promoter region of the target gene. The insertion leads to disruption of the open reading frame or interferes with the regulation of gene expression and thus blocks or reduces the expression of the target gene. For example, RNA according to the present invention can preferably be obtained by using vectors containing an expression cassette. This vector DNA can be transferred into the plant or plant cell and expresses the RNA.

According to a preferred embodiment of the invention the inhibition of the Lewis-type β1,3-galactosyltransferase is performed by insertional mutagenesis either directed or by random insertion into the plant genome.

The inhibition of the Lewis-type β1,3-galactosyltransferase according to the present invention may also be performed by physical or chemical mutagenesis. Physical mutagenesis includes, but is not limited to gamma and fast neutron irradiation of seeds. Chemical mutagenesis includes, but is not limited to treatment of seeds with ethyl-methanesulfonate leading to point mutations, which inactivate glycosyltransferases (Strasser et al., 2005, Biochem. J., 387: 385-391).

According to another preferred embodiment of the invention the inhibition of the Lewis-type β1,3-galactosyltransferase is performed by “Targeting Induced Local Lesions IN Genomes” (TILLING), a method whereby chemical mutagenesis followed by screening for single-base changes to discover induced mutations that alter protein function is used (described by Henikoff et al., 2004, Plant Physiol. 135: 630-636).

According to another aspect of the present invention, there is provided a method of preparing a cDNA comprising the nucleic acid molecule, in particular the DNA of the invention, wherein RNA is isolated from a plant cell, in particular from stem or silique cells, by means of which a reverse transcription is carried out after the addition of a reverse transcriptase and primers. The individual steps of this method are carried out according to protocols known per se. For the reverse transcription, on the one hand, it is possible to produce the cDNA of the entire mRNA with the help of oligo (dT) primers, and only then to carry out a PCR by means of selected primers so as to prepare nucleic acid molecules comprising the Lewis-type β1,3-galactosyltransferase. On the other hand, the selected primers may directly be used for the reverse transcription so as to obtain short, specific cDNA. The suitable primers may be prepared e.g. synthetically according to the pattern of cDNA sequences of the transferase.

The invention furthermore relates to a method of cloning a plant Lewis-type β1,3-galactosyltransferase, characterized in that the nucleic acid molecule of the invention is cloned into a vector which subsequently is transfected into a host cell or host, respectively, wherein, by selection and amplification of transfected host cells, cell lines are obtained which express the active Lewis-type β1,3-galactosyltransferase. The DNA molecule is inserted into the vector by aid of restriction endonucleases. For the vector, there applies what has already been said above. What is important in this method is that an efficient host-vector system is chosen. To obtain an active enzyme, eukaryotic host cells are particularly suitable. One possible way is to transfect the vector in insect cells. In doing so, in particular an insect virus would have to be used as vector, such as, e.g., baculovirus.

Of course, plants or plant cells, human or other vertebrate cells, generally “organisms”, can also be transfected, in which case the latter would express an enzyme foreign to them.

Preferably, a method of preparing recombinant host cells, in particular plant cells or plants, respectively, with a suppressed or completely stopped Lewis-type β1,3-galactosyltransferase production is provided, which is characterized in that at least one of the vectors according to the invention, i.e. that one comprising the inventive nucleic acid molecule, the mutant nucleic acid molecule or the nucleic acid molecule coding for ribozymes or the one comprising the nucleic acid molecule in inverse orientation to the promoter, or the one comprising the nucleic acid molecule in sense and antisense orientation is inserted into the host cell or plant, respectively. What has been said above for the transfection also is applicable in this case.

Also provided is the method of producing a plant or plant cells having blocked or reduced expression of endogenous Lewis-type β1,3-galactosyltransferase present in the wildtype plant or plant cell at the transcription or translation level, wherein the plant or plant cell is stably or transiently transfected by a method as mentioned herein. The plant or plant cell is then cultivated. Preferably, the reduction is in amounts of at least 10%, at least 20%, at least 50%, as compared to the wildtype plant or plant cell. Specifically preferred are plant or plant cells which do not exhibit the endogenous Lewis-type β1,3-GalT activity of the wild type plant or plant cell (i.e. a reduction of the endogenous Lewis-type β1,3-GalT activity to under 10%, preferably to under 1%, of the wild type). Even more preferred are plants or plant cells wherein the reduction in Lewis-type β1,3-GalT activity results in no activity at all (i.e. an activity which is below the detection limit). This may be achieved e.g. with “knock-out” mutants where this gene is completely silenced by methods using recombinant nucleic acid technology.

This method can also be used, to prevent the expression of the Lewis-A epitope in the plant or plant cell which is capable of producing complex N-glycans after the transfection.

As host cells, plant cells may, e.g., be used, wherein, e.g., the Ti plasmid with the agrobacterium system is eligible. With the agrobacterium system it is possible to transfect a plant directly: agrobacteria cause root stem galls in plants. If agrobacteria infect an injured plant, the bacteria themselves do not get into the plant, but they insert the recombinant DNA portion, the so-called T-DNA, from the annular, extrachromosomal, tumor-inducing Ti-plasmid into the plant cells. The T-DNA, and thus also the DNA molecule inserted therein, are installed in the chromosomal DNA of the cell in a stable manner so that the genes of the T-DNA will be expressed in the plant. There exist numerous known, efficient transfection mechanisms for different host systems. Some examples are electroporation, the calcium phosphate method, microinjection and the liposome method. Subsequently, the transfected cells are selected, e.g. on the basis of antibiotic resistances for which the vector comprises genes, or other marker genes. Then the transfected cell lines are amplified, either in small amounts, e.g. in Petri dishes, or in large amounts, e.g. in fermentors. Furthermore, plants have a particular characteristic, i.e. they are capable to redevelop from one (transfected) cell or from a protoplast; respectively, to a complete plant which can be grown. Depending on the vector used, processes will occur in the host so that the enzyme expression will be suppressed or completely blocked: If the vector comprising the DNA molecule with the deletion, insertion or substitution mutation is transfected, a homologous recombination will occur: the mutant DNA molecule will recognize the identical sequence in the genome of the host cell despite its mutation and will be inserted exactly on that place so that a “knockout gene” is formed. In this manner, a mutation is introduced into the gene for the Lewis-type β1,3-galactosyltransferase which is capable of inhibiting the faultless expression of the Lewis-type β1,3-galactosyltransferase enzyme. As has been explained above, with this technique it is important that the mutation suffices to block the expression of the active protein. After selection and amplification, the gene may be sequenced as an additional check so as to determine the success of the homologous recombination or the degree of mutation, respectively.

In case the vector comprises the inventive DNA molecule in sense or inverse direction to the promoter, a sense or antisense-mRNA will be expressed in the transfected cell (or plant, respectively). The antisense mRNA is complementary at least to a part of the mRNA sequence of the Lewis-type β1,3-galactosyltransferase and may likewise inhibit translation of the enzyme or lead to degradation of the target mRNA. As an example of a method of suppressing the expression of a gene by antisense technique, reference is made to the publication by Smith et al., 1990, Mol. Gen. Genet., 224: 477-481, wherein in this publication the expression of a gene involved in the maturing process of tomatoes is inhibited. Double-stranded RNA (dsRNA) has recently been shown to trigger sequence-specific gene silencing in a wide variety of organisms, including plants (for review see Watson et al., 2005, FEBS Letters, 579: 5982-5987).

In all the systems, expression of the Lewis-type β1,3-galactosyltransferase is at least suppressed, preferably even completely blocked. The degree of the disturbance of the gene expression will depend on the degree of complexing, homologous recombination, on possible subsequent coincidental mutations and on other processes in the region of the genome. The transfected cells are checked for Lewis-type β1,3-galactosyltransferase activity and selected.

Moreover, it is possible to still further increase the above described suppression of the expression of the Lewis-type β1,3-galactosyltransferase by introducing into the host a vector comprising a gene coding for a β1,4-galactosyltransferase (e.g. from human or any other source) in addition to the insertion of an above described vector. The attachment of β1,3-galactose may be reduced by the action of other enzymes from any other source, the combination of the inhibition of the expression of an active Lewis-type β1,3-galactosyltransferase by means of the inventive vector and by means of a mammalian enzyme vector being particularly efficient.

Preferably, the transformation of plants, which lack an active Lewis-type β1,3-galactosyltransferase with mammalian glycosylation enzymes includes in particular but not exclusively human β1,4-galactosyltransferase, α2,6-sialyltransferase, α2,3-sialyltransferase, β1,4-N-acetylglucosaminyltransferase III, β1,4-N-acetylglucosaminyltransferase IV, β1,6-N-acetylglucosaminyltransferase V and β1,4-N-acetylglucosaminyltransferase VI or any combination of these enzymes. Any plant species may be used for transfection, including but not limited to alfalfa, apple, Arabidopsis thaliana, cotton, common bean, maize, mung bean, poplar, potato, rice, soybean, tobacco, grapewine, tomato plant, wheat, barley, Nicotiana benthamiana, lemna, lettuce, spinach, sugarcane, banana, cowpea, papaya, carrot or Brassica.

In a preferred embodiment normal expression of the Lewis-A epitope is prevented in at least one organ or tissue of the plant, and the plant or plant cell comprises a dysfunctional β1,2-xylosyltransferase and/or a dysfunctional α1,3-fucosyltransferase. The Lewis-type β1,3-galactosyltransferase is at least suppressed, preferably even completely blocked in plants that have reduced amounts of complex N-glycans carrying β1,2-linked xylose and/or core α1,3-linked fucose, preferably those plants even lack completely complex N-glycans with β1,2-linked xylose and/or core α1,3-linked fucose. The generation of such plants has been described (Strasser et al., 2004, FEBS Lett., 561: 132-136).

A specifically preferred embodiment according to the present invention is a plant or plant cell wherein the β1,3-GalT activity is reduced or erased in addition to a reduction or complete inactivation of β1,2-xylosyltransferase and core α1,3-fucosyltransferase and wherein at least one, preferably at least two, especially at least three mammalian glycosyltransferase activities have been introduced.

In yet another embodiment of the invention the Lewis-A epitope is at least reduced, preferably even completely absent from plants that have in addition reduced amounts of complex N-glycans carrying β3,2-linked xylose and core α1,3-linked fucose, preferably those plants even lack completely complex N-glycans with β3,2-linked xylose and core α1,3-linked fucose. The generation of such plants, which can still produce complex N-glycans with terminal GlcNAc residues, has been described (Strasser et al., 2004, FEBS Lett., 561: 132-136) and the generation of such plants by knockout or gene silencing is known to the skilled artisan.

Provided in another aspect is a transformed plant or plant cell with an increased expression of a Lewis-type β1,3-galactosyltransferase, comprising a DNA molecule with a sequence of an active Lewis-type β1,3-galactosyltransferase as described above.

Alternatively a transformed plant or plant cell with a reduced or no expression of a Lewis-type β1,3-galactosyltransferase is provided, comprising a DNA molecule, a vector or a RNA which interfere with the expression of a Lewis-type β1,3-galactosyltransferase or are used to knock-out the Lewis-type β1,3-galactosyltransferase.

Another advantageous method of producing recombinant host cells, in particular plant cells, or plants, respectively, consists in that the DNA molecule comprising the mutation is inserted into the genome of the host cell, or plant, respectively, in the place of the non-mutated homologous sequence (Schaefer et al., 1997, Plant J.; 11: 1195-1206). This method thus does not function with a vector, but with a pure DNA molecule. The DNA molecule is inserted into the host e.g. by gene bombardment, microinjection or electroporation, to mention just three examples. As has already been explained, the DNA molecule binds to the homologous sequence in the genome of the host so that a homologous recombination and thus reception of the deletion, insertion or substitution mutation, respectively, will result in the genome: Expression of the Lewis-type β1,3-galactosyltransferase can be suppressed or completely blocked, respectively.

Preferably, recombinant plants or plant cells, respectively, are provided which have been prepared by one of the methods described above, their Lewis-type β1,3-galactosyltransferase production being suppressed or completely blocked, respectively. Preferably, their Lewis-type β1,3-galactosyltransferase activity is less than 50%, in particular less than 20% or less than 10%, particularly preferred 0%, of the Lewis-type β1,3-galactosyltransferase activity occurring in natural plants or plant cells, respectively. The advantage of these plants or plant cells, respectively, is that the glycoproteins produced by them do not comprise any or hardly comprise any β1,3-galactose linked to glycoprotein N-glycans and do not comprise any or hardly comprise any Lewis-A type containing N-glycans. If products of these plants are taken up by or delivered to human or vertebrate bodies, there will be no immune reaction due to the plant Lewis-A epitope or due to the β1,3-linked galactose.

Preferably, recombinant plants or plant cells, respectively, are provided which have been prepared by one of the methods described above, their Lewis-type β1,3-galactosyltransferase production being suppressed or completely blocked, respectively. Preferably, their proportion of Lewis-A epitope containing N-glycans is less than 50%, in particular less than 20%, particularly preferred 0%, of the Lewis-A epitope containing N-glycans occurring in natural plants or plant cells, respectively. The advantage of these plants or plant cells, respectively, is that the glycoproteins produced by them do not comprise any or hardly comprise any β1,3-galactose linked to glycoprotein N-glycans and do not comprise any or hardly comprise any Lewis-A type containing N-glycans. Further advantage of these plants compared to other glycosylation mutants (e.g. von Schaewen et al., 1993, Plant Physiol., 102: 1109-1118) is that the glycosylation is not blocked at an early stage in the pathway and complex N-glycans can be formed and elongated by the introduction of mammalian glycosyltransferases as described above (e.g. β1,4-galactosyltransferase, sialyltransferase). Glycoproteins from these plants comprise a N-glycosylation pattern, which is more comparable to mammalian-type glycosylation than glycoproteins from wildtype plants or plant cells.

A further aspect relates to a method of preparing plants or plant cells, respectively, which comprise changes in β1,3-galactose linked to glycoprotein N-glycans due to interfering with the protein targeting and localization of the Lewis-type β1,3-galactosyltransferase within the plant cell, e.g. by retention of the Lewis-type β1,3-galactosyltransferase in the endoplasmic reticulum due to overexpression or elimination of proteins, which are required for proper transport to the site of action (e.g. Golgi apparatus) (e.g. Nilson et al., 1994, 13: 562-574). For example prevention of proper targeting can be achieved by overexpression of the cytoplasmic tail of either the β1,3-galactosyltransferase or of another glycosyltransferase, which interacts with the Lewis-type β1,3-galactosyltransferase (e.g. Milland et al., 2002, J. Biol. Chem., 277: 10374-10378).

The transformed plant or plant cell with increased or reduced expression of the Lewis-type β1,3-galactosyltransferase preferably comprises a nucleotide sequence linked to an exogenous promoter that drives expression in said plant or plant cell, respectively, wherein said nucleotide sequence encodes a functional mammalian galactosyltransferase that is expressed in the plant or plant cell, respectively. The mammalian galactosyltransferase is preferably a β1,4-galactosyltransferase, especially preferred a human β1,4-galactosyltransferase.

Furthermore in another embodiment the transformed plant or plant cell comprises a nucleotide sequence linked to an exogenous promoter that drives expression in said plant or plant cell, respectively, wherein said nucleotide sequence encodes a functional mammalian N-acetylglucosaminyltransferase that is expressed in the plant or plant cell, respectively. Preferably, the mammalian N-acetylglucosaminyltransferase is a β1,2-N-acetylglucosaminyltransferase I or II, preferably a human β1,2-N-acetylglucosaminyltransferase I or II, or a β1,4-N-acetylglucosaminyltransferase III or IV, preferably a human β1,4-N-acetylglucosaminyltransferase III or IV, or a β1,6-N-acetylglucosaminyltransferase V, preferably a human β1,6-N-acetylglucosaminyltransferase V.

Additionally the transformed plant or plant cell may comprise a nucleotide sequence linked to an exogenous promoter that drives expression in said plant or plant cell, respectively, wherein said nucleotide sequence encodes a functional mammalian sialyltransferase that is expressed in the plant or plant cell, respectively. Preferably, the mammalian sialyltransferase is a human or rat α2,6-sialyltransferase or a human or rat α2,3-sialyltransferase.

In another aspect the invention provides a method of producing recombinant mammalian glycoproteins or glycosylated polypeptides, wherein a transformed plant or plant cell expresses the glycoprotein or polypeptide and a plant is transfected with a nucleic acid that encodes the glycoprotein or polypeptide. The plant can be transfected with the nucleic acid encoding the glycoprotein or polypeptide before or after the plant or plant cell is transformed for increased or reduced expression of the Lewis-type β1,3-galactosyltransferase. Preferably the glycoprotein is a human antibody or a fragment thereof, preferably a human immunoglobulin G (IgG) and wherein the fragment is preferably a Fab, Fab′, Fab₂ or scFv (single chain variable fragment) fragment.

Provided is the method of preparing recombinant glycoproteins which is characterized in that the inventive, recombinant plants or plant cells, respectively, whose Lewis-type β1,3-galactosyltransferase production is suppressed or completely blocked, respectively, or plants or cells, respectively, in which the Lewis-A epitope formation is partially or completely blocked according to the method of the invention, are transfected with the gene coding for any glycoprotein of interest so that the recombinant glycoproteins are expressed. In doing so, as has already been described above, vectors comprising genes for the desired proteins are transfected into the host plant or host plant cells, respectively, as has also already been described above. The transfected plant cells will express the desired proteins, and they have no or hardly any β1,3-galactose linked to glycoprotein N-glycans. Thus, they do not trigger potentially immune reactions directed against β1,3-galactose linked to glycoprotein N-glycans or against the Lewis-A epitope linked to N-glycans and provide a mammalian-like N-glycosylation, which is preferably for therapeutic proteins. Any proteins may be produced in these systems.

Advantageously, a method of preparing recombinant human glycoproteins is provided which is characterized in that the recombinant plants or plant cells, respectively, whose Lewis-type β1,3-galactosyltransferase production is suppressed or completely blocked, respectively, or plants or cells, respectively, in which the Lewis-A epitope formation is partially or completely blocked according to the method of the invention, are transfected with the gene that expresses the glycoprotein so that the recombinant glycoproteins are expressed. By this method it becomes possible to produce human or other mammalian proteins in plants (plant cells) which, if taken up by the human or other mammalian body, do not trigger any immune reaction directed against β1,3-galactose linked to glycoprotein N-glycans or against the Lewis-A epitope linked to N-glycans. There, it is possible to utilize plant types for producing the recombinant glycoproteins which serve as food stuffs, e.g. banana, potato and/or tomato.

The tissues of this plant comprise the recombinant glycoprotein so that, e.g. by extraction of the recombinant glycoprotein from the tissue and subsequent administration, or directly by eating the plant tissue, respectively, the recombinant glycoprotein is taken up in the human or other mammalian body.

Preferably, a method of preparing recombinant human glycoproteins for medical or veterinary medical use is provided, wherein the inventive, recombinant plants or plant cells, respectively, whose Lewis-type β1,3-galactosyltransferase production is suppressed or completely blocked, respectively, or plants or cells, respectively, in which the Lewis-A epitope formation is partially or completely blocked according to the method of the invention, are transfected with the gene that expresses the glycoprotein so that the recombinant glycoproteins are expressed. In doing so, any protein can be used which is of medical or veterinary medical interest including, but not limited to antibodies, hormones, growth factors. It is also possible to use non-glycoproteins in which a N-glycosylation site has been introduced (e.g. by recombinant DNA technology) into the wildtype sequence and which therefore have been transformed from non-glycosylated proteins to a glycoprotein (“artificial glycoproteins”).

Preferably the glycoprotein or glycosylated polypeptide is selected from the group consisting of human insulin, preproinsulin, proinsulin, glucagon, interferons such as alpha-interferon, beta-interferon, gamma-interferon, blood-clotting factors selected from Factor VII, VIII, IX, X, XI, and XII, fertility hormones including luteinising hormone, follicle stimulating hormone growth factors including epidermal growth factor, platelet-derived growth factor, granulocyte colony stimulating, prolactin, oxytocin, thyroid stimulating hormone, adrenocorticotropic hormone, calcitonin, parathyroid hormone, somatostatin, erythropoietin (EPO), haemoglobin, serum albumin, collagen and enzymes such as beta-glucocerebrosidase and human and non-human proteins selected from amidases, amylases, carbohydrases, cellulase, dextranase, esterases, glucanases, glucoamylase, lactase, lipases, pepsin, peptidases, phytases, proteases, pectinases, casein, whey proteins, soya proteins, gluten and egg albumin. Of course, the present invention is preferably applied on glycoproteins which are native glycoproteins (i.e. which are glycosylated in their physiological or physiologically active form).

Moreover, the present invention relates to recombinant glycoproteins according to a method described above, wherein they have been prepared in plant systems and wherein their peptide sequence comprises less than 50%, in particular less than 20%, particularly preferred 0%, of the β1,3-bound galactose residues occurring in proteins expressed in non-Lewis-type β1,3-galactosyltransferase-reduced plant systems. Naturally, glycoproteins which do not comprise β1,3-linked galactose residues are to be preferred. The amount of the β1,3-linked galactose and the amount of the Lewis-A epitope will depend on the degree of the above described suppression of the Lewis-type β1,3-galactosyltransferase.

Preferably, the invention relates to recombinant human glycoproteins which have been produced in plant systems according to a method described above and whose peptide sequence comprises less than 50%, in particular less than 20%, particularly preferred 0%, of the β1,3-bound galactose residues occurring in the proteins expressed in natural, non-Lewis-type β1,3-galactosyltransferase-reduced, plant systems.

Preferably, the invention relates to recombinant human glycoproteins which have been produced in plant systems according to a method described above and whose peptide sequence comprises less than 50%, in particular less than 20%, particularly preferred 0%, of the Lewis-A epitope occurring in the proteins expressed in natural, non-Lewis-type β1,3-galactosyltransferase-reduced, plant systems.

A particularly preferred embodiment relates to recombinant human glycoproteins for medical or veterinary medical use which have been prepared in plant systems according to a method described above and whose peptide sequence comprises less than 50%, in particular less than 20%, particularly preferred 0%, of the β1,3-bound galactose residues occurring in the proteins expressed in non-Lewis-type β1,3-galactosyltransferase-reduced plant systems.

A particularly preferred embodiment relates to recombinant human glycoproteins for medical or veterinary medical use which have been prepared in plant systems according to a method described above and whose peptide sequence comprises less than 50%, in particular less than 20%, particularly preferred 0%, of the Lewis-A epitope occurring in the proteins expressed in natural, non-Lewis-type β1,3-galactosyltransferase-reduced, plant systems.

The glycoproteins according to the invention may include other bound oligosaccharide units specific for plants, whereby in the case of human glycoproteins they differ from these natural glycoproteins. Nevertheless, by the glycoproteins according to the invention, a slighter immune reaction or no immune reaction at all, respectively, is triggered in the human or other mammalian body, since, as has already been explained in the introductory portion of the specification, the β1,3-linked galactose residue, together with α1,4-linked fucose residues, could lead to immune reactions or cross immune reaction, respectively.

The glycoproteins according to the invention may include other bound oligosaccharide units specific for plants, whereby—in the case of human glycoproteins—they differ from these natural glycoproteins. Nevertheless, by the glycoproteins according to the invention, a better efficacy is triggered in the human body, since, as has already been explained in the introductory portion of the specification, the β1,3-linked galactose residue, together with α1,4-linked fucose residues, are usually not present on mammalian N-linked glycans.

A further aspect comprises a pharmaceutical composition comprising the glycoproteins according to the invention. In addition to the glycoproteins of the invention, the pharmaceutical composition comprises further additions common for such compositions.

These are, e.g., suitable diluting agents of various buffer contents (e.g. Tris-HCl, acetate, phosphate, pH and ionic strength), additives, such as tensides and solubilizers (e.g. Tween 80, Polysorbate 80), preservatives (e.g. Thimerosal, benzyl alcohol), adjuvants, antioxidants (e.g. ascorbic acid, sodium metabisulfite), emulsifiers, fillers (e.g. lactose, mannitol), covalent bonds of polymers, such as polyethylene glycol, to the protein, incorporation of the material in particulate compositions of polymeric compounds, such as polylactic acid, polyglycolic acid, etc. or in liposomes, auxiliary agents and/or carrier substances which are suitable in the respective treatment. Such compositions will influence the physical condition, stability, rate of in vivo liberation and rate of in vivo excretion of the glycoproteins of the invention.

The invention also relates to a method of preparing “plantified” carbohydrate units of human and other vertebrate glycoproteins or other glycoconjugates, wherein UDP-galactose as well as plant Lewis-type β1,3-galactosyltransferase encoded by an above-described DNA molecule are added to a sample that comprises a carbohydrate unit or a glycoprotein, respectively, so that galactose in β1,3-position is transferred by the plant Lewis-type β1,3-galactosyltransferase to the carbohydrate unit or to the glycoprotein, respectively. By the method according to the invention for cloning of plant Lewis-type β1,3-galactosyltransferase it is possible to produce large amounts of purified enzyme. To obtain a fully active transferase, suitable reaction conditions are provided.

The invention also relates to a method of overexpression of the plant Lewis-type β1,3-galactosyltransferase in plants. The overexpression of the plant Lewis-type β1,3-galactosyltransferase leads to increased amounts of Lewis-A epitopes in plants. Overexpression includes but is not limited to expression in tissues, like leaves, which in natural plants have limited amounts of Lewis-A epitopes. Overexpression in plants can be achieved by using a constitutive promoter, which includes but is not limited to the cauliflower mosaic virus 35S (CaMV35S) promoter and the nopaline synthase (nos) promoter, or the use of tissue-specific promoter like for example the seed-specific promoter arcelin5-I from common bean (DeJaeger et al., 2002, Nature Biotechn., 20: 1265-1268). These techniques are well known to the skilled artisan. Overexpression of the Lewis-A epitope can be detected on glycoproteins from plants by immunoblots using JIM84 antibodies (Horsley et al., 1993, Journal of Exp. Botany 44, Supplement: 223-229), which are specific for the plant Lewis-A epitope (Fitchette et al., 1999, Plant Physiol. 121: 333-343) and subsequently by total N-glycan analysis using mass spectrometry (Wilson et al., 2001, Glycobiology 11: 261-274).

The invention also relates to a method of overexpression of Lewis-type β1,3-galactosyltransferase in plants, whereby the Lewis-type β1,3-galactosyltransferase is derived from bryophytes such as Physcomitrella patens. The overexpression of the bryophyte Lewis-type β1,3-galactosyltransferase leads to increased amounts of Lewis-A epitopes in plants. For overexpression the same promoters as described above are suitable.

The invention also relates to a method of overexpression of animal β1,3-galactosyltransferase in plants. The overexpression of the animal (vertebrate or non-vertebrate) β1,3-galactosyltransferase leads to increased amounts of Lewis-A epitopes in plants. Preferably, a mammalian β1,3-galactosyltransferase is expressed in plants, which includes but is not limited to β1,3-galactosyltransferase I, β1,3-galactosyltransferase II, β1,3-galactosyltransferase III, β1,3-galactosyltransferase IV, β1,3-galactosyltransferase V, β1,3-galactosyltransferase VI and β1,3-galactosyltransferase VII (Amado et al., 1999, Biochim Biophys Acta., 1473: 35-53; Hennet, 2002, Cell. Mol. Life. Sci. 59: 1081-1095). In another embodiment of the invention preferably, an invertebrate β1,3-galactosyltransferase is expressed in plants, which includes but is not limited to core 1 galactosyltransferases (Muller et al., 2005, FEBS J.; 272: 4295-4305). For overexpression the same promoters as described above are suitable. In another embodiment of the invention preferably a bacterial β1,3-galactosyltransferase (Appelmelk et al., 2000, Infect. Immun. 68: 5928-5932; Gilbert et al., 2000, J. Biol. Chem. 275, 3896-3906) is expressed in plants.

The invention also provides a method of producing at least a plant or a plant cell wherein the plant Lewis-type β1,3-galactosyltransferase activity is increased that comprises introducing into the plant or plant cell a nucleic acid sequence encoding a plant Lewis-type β1,3-galactosyltransferase nucleotide sequence, mammalian β1,3-galactosyltransferase nucleotide sequence, or bryophyte Lewis-type β1,3-galactosyltransferase nucleotide sequence.

The invention further relates to a transformed plant or plant cell with increased expression of the Lewis-A epitope, characterized in that the plant or plant cell comprises a mammalian β1,3-galactosyltransferase nucleotide sequence, preferably selected from β1,3-galactosyltransferase 1, β1,3-galactosyltransferase 2 or β1,3-galactosyltransferase 5, a bryophyte Lewis-type β1,3-galactosyltransferase nucleotide sequence or a plant Lewis-type β1,3-galactosyltransferase nucleotide sequence.

The invention will be explained in more detail by way of the following examples and drawing figures to which, of course, it shall not be restricted. In detail, in the drawings,

FIG. 1: shows typical plant Lewis-A type N-glycans and type 1 chain containing N-glycan structures;

FIG. 2: shows a schematic presentation of the plant N-glycosylation pathway;

FIG. 3: shows a schematic presentation of the enzymatic reaction catalysed by a plant Lewis-type β1,3-galactosyltransferase;

FIG. 4: shows the cDNA (SEQ ID NO: 1) encoding a plant Lewis-type β1,3-galactosyltransferase from A. thaliana.

FIG. 5: shows the amino acid sequence (SEQ ID NO: 2) of the plant Lewis-type β1,3-galactosyltransferase derived therefrom;

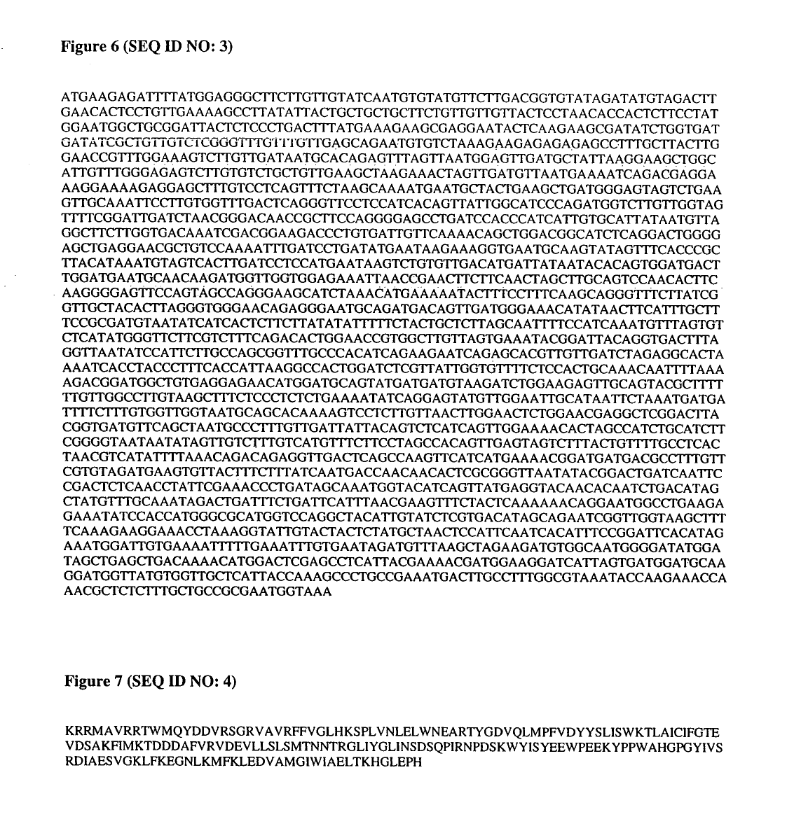

FIG. 6: shows the genomic sequence (SEQ ID NO: 3) of the plant Lewis-type β1,3-galactosyltransferase;

FIG. 7: shows the amino acid sequence (aa 406-597 of SEQ ID No: 2) of the putative catalytic domain (SEQ ID NO: 4) of plant Lewis-type β1,3-galactosyltransferase from A. thaliana;

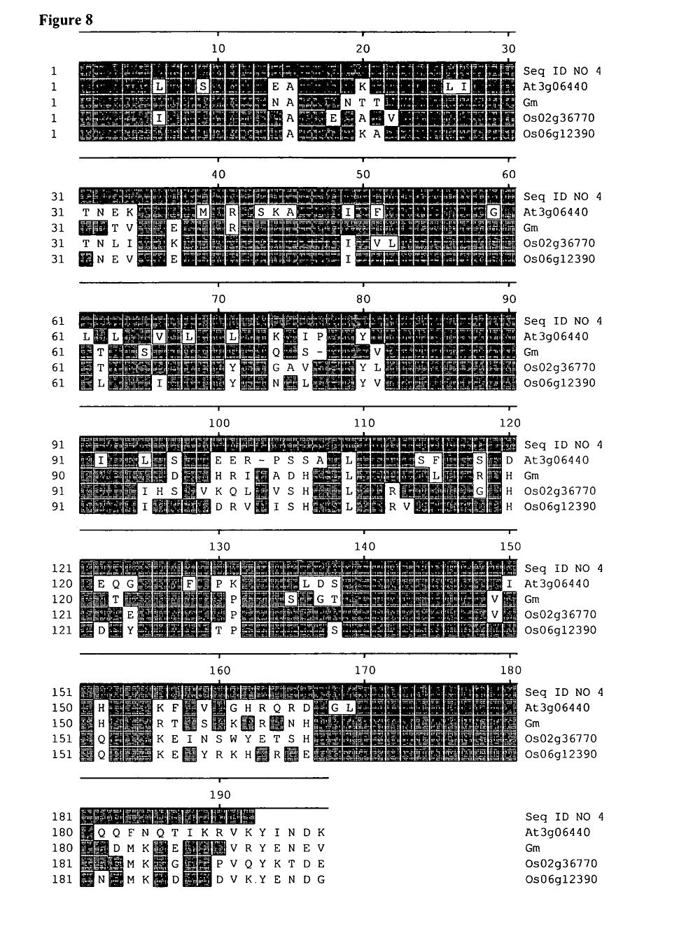

FIG. 8: shows the amino acid alignment of SEQ ID NO: 4 with one homologous sequence from A. thaliana (At3g06440) and two rice (Os02g36770, Os06 g12390) and one soybean (Gm) sequence;

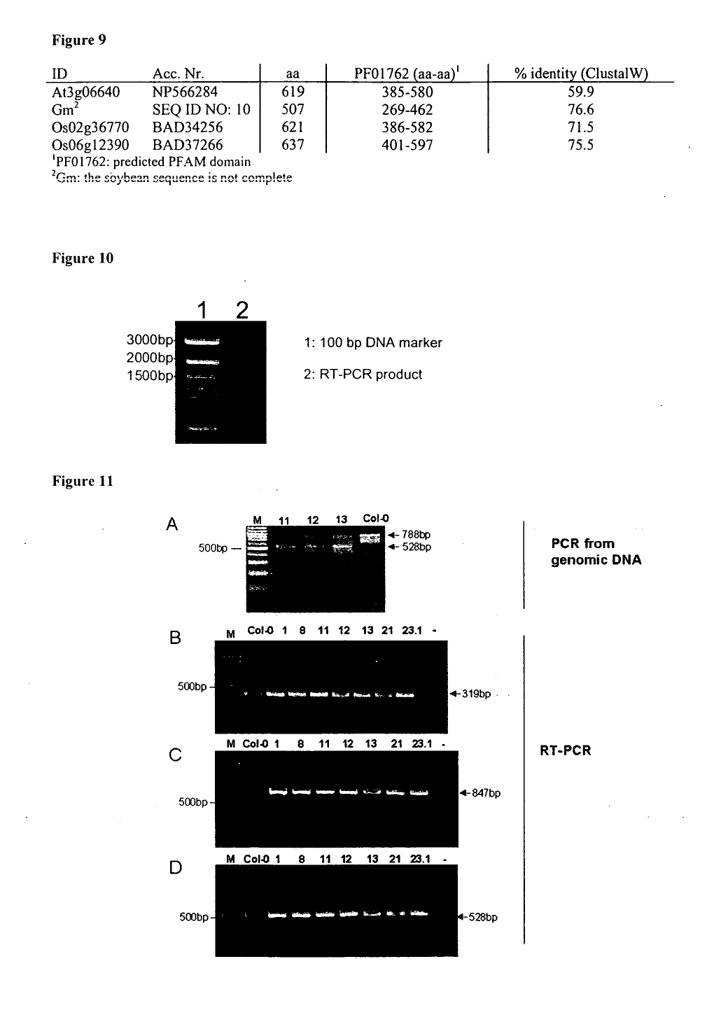

FIG. 9: shows a table, which displays the amino acid sequence identity between SEQ ID NO: 4 and the predicted catalytic domain of putative plant Lewis-type β1,3-galactosyltransferases;

FIG. 10: shows the RT-PCR amplification product of SEQ ID NO: 1 from A. thaliana siliques;

FIG. 11: shows the PCR amplification from genomic DNA isolated from plants transformed with the plant Lewis-type β1,3-galactosyltransferase and RT-PCR from RNA extracted from those plants (Col-0: wildtype);

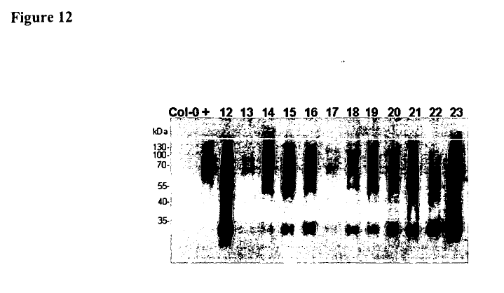

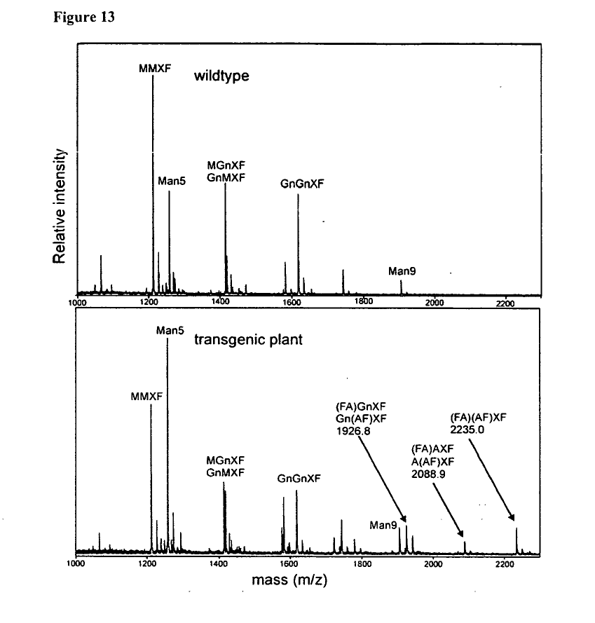

FIG. 12: shows a comparison by immunoblot analysis of the plant Lewis-type β1,3-galactosyltransferase activity of plants expressing the plant Lewis-type β1,3-galactosyltransferase from A. thaliana with that of a control (untransformed wildtype plant: Col-0);

FIG. 13: shows a comparison by mass (mass spectra) of plants expressing the plant Lewis-type β1,3-galactosyltransferase from A. thaliana (transgenic plant) with that of a control (wildtype);

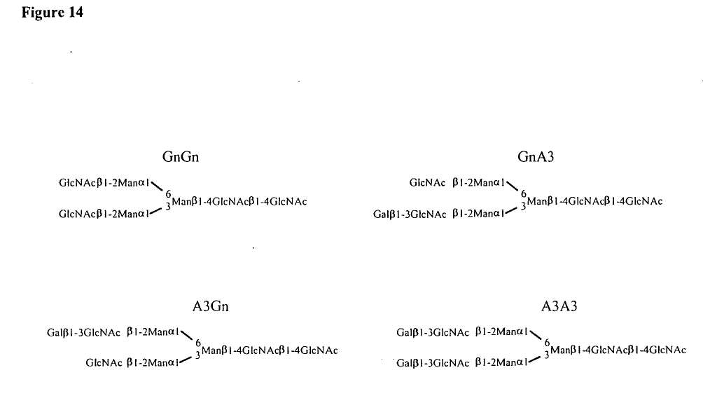

FIG. 14: shows the structure of the acceptor substrate (GnGn) and products (A3Gn/GnA3/A3A3) of the plant Lewis-type β1,3-galactosyltransferase from A. thaliana suitable for an assay;

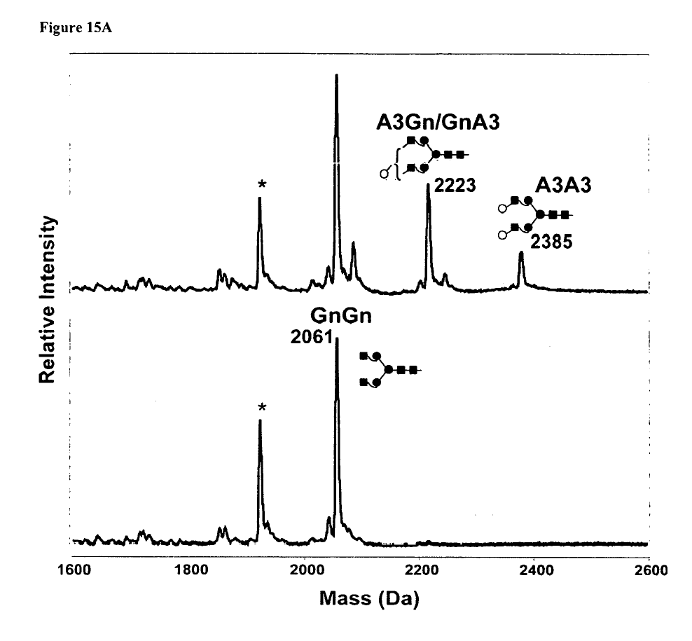

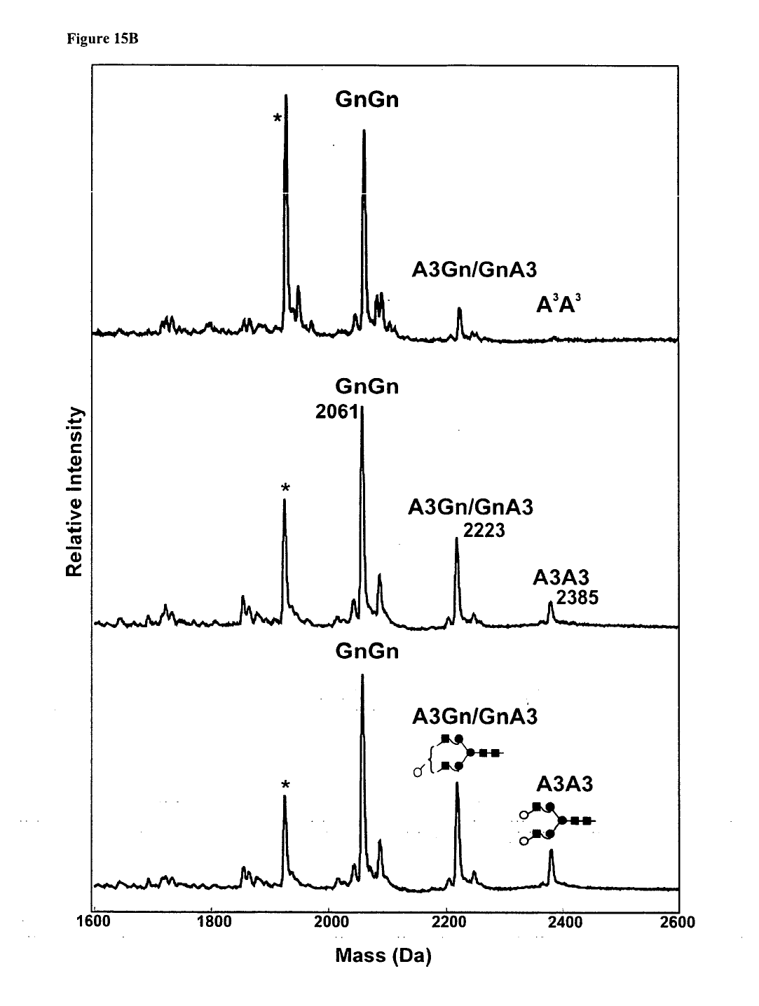

FIG. 15: shows the analysis of the plant Lewis-type β1,3-galactosyltransferase product by mass spectrometry: (A) lower panel: only substrate (GnGn see FIG. 14 for detailed structures), upper panel product after incubation with Lewis-type β1,3-galactosyltransferase; (B) treatment with different β-galactosidases, lower panel: untreated, middle panel treatment with β-galactosidase from Aspergillus oryzae, upper panel: digestion with β-galactosidase from bovine testis;

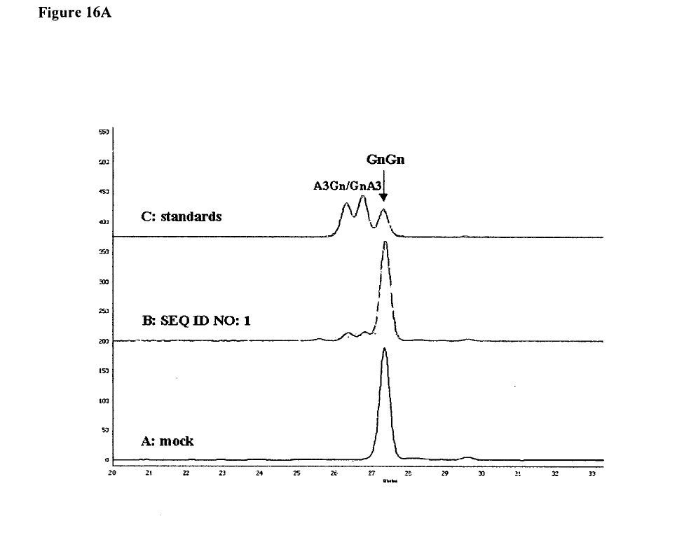

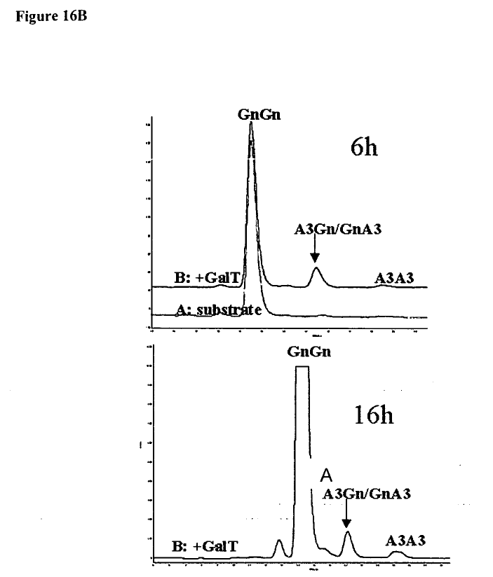

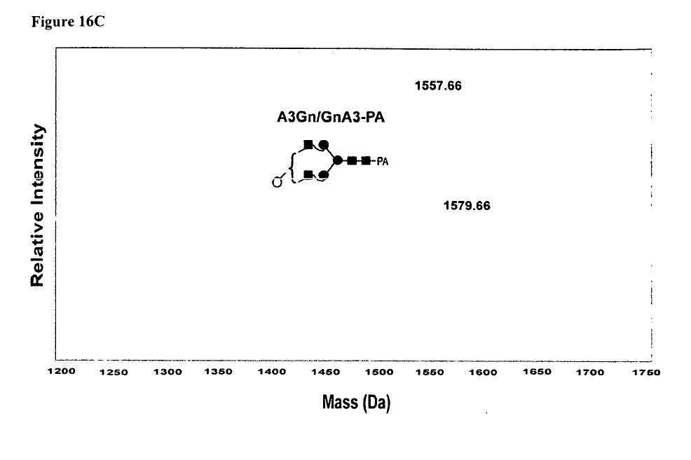

FIG. 16: shows the analysis of the plant Lewis-type β1,3-galactosyltransferase product by reversed-phase HPLC (A) and normal phase HPLC (B), the additional peak from the normal phase HPLC was collected and analyzed by MALDI-TOF MS (C);

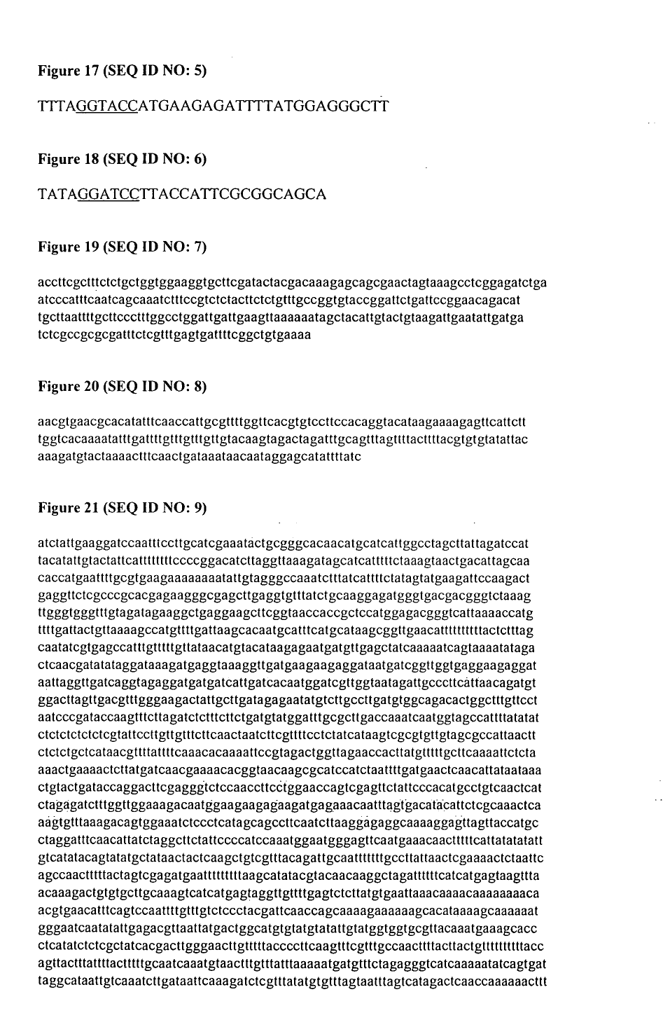

FIG. 17: shows the forward primer for ORF amplification (SEQ ID NO: 5);

FIG. 18: shows the reverse primer for ORF amplification (SEQ ID NO: 6);

FIG. 19: shows the 5′ untranslated region (SEQ ID NO: 7) of the Arabidopsis Lewis-type β1,3-galactosyltransferase;

FIG. 20: shows the 3′ untranslated region (SEQ ID NO: 8) of the Arabidopsis Lewis-type β1,3-galactosyltransferase;

FIG. 21: shows the 2481 bp promoter region (SEQ ID NO: 9) of the Arabidopsis Lewis-type β1,3-galactosyltransferase;

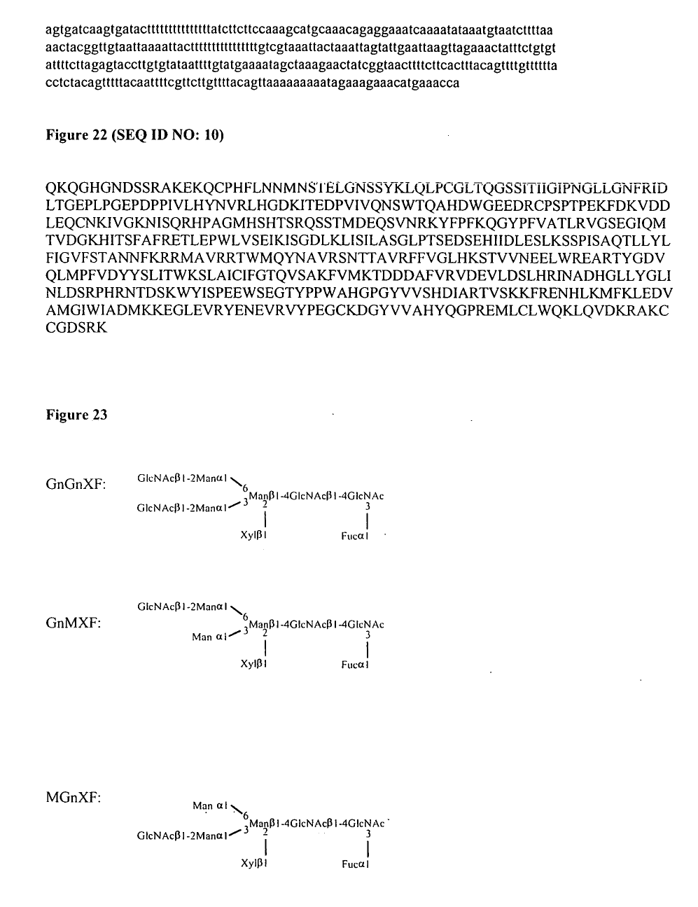

FIG. 22: shows the C-terminal amino acid sequence of a putative soybean (Glycine max) Lewis-type 1,3-galactosyltransferase (SEQ ID NO: 10);

FIG. 23: shows typical complex-type N-glycans structures;

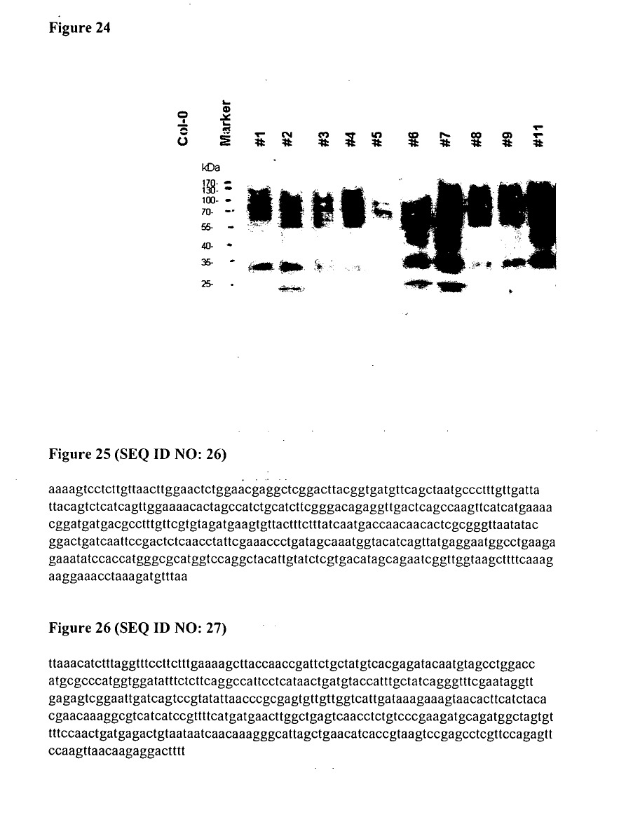

FIG. 24: shows a comparison by immunoblot analysis of the β1,3-galactosyltransferase activity of plants expressing a murine Lewis-type β1,3-galactosyltransferase with that of a control;

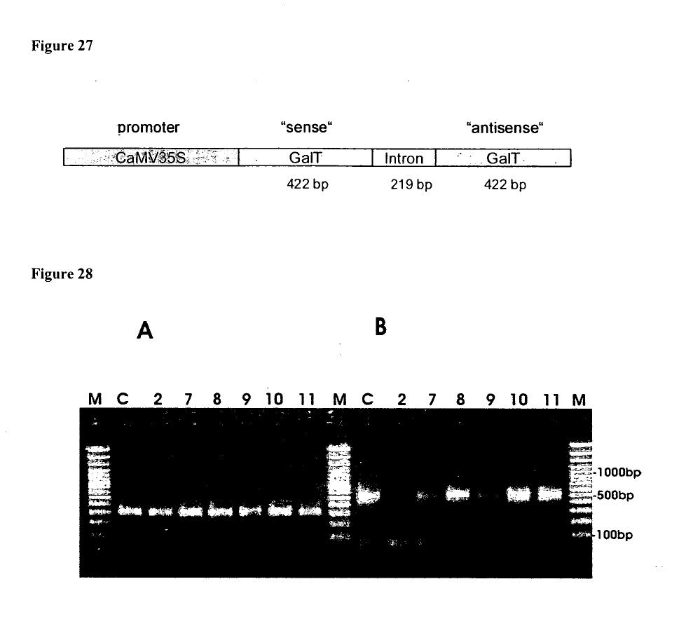

FIG. 25: shows the sense cDNA (SEQ ID NO: 26), which is present in the RNAi construct as shown in FIG. 27;

FIG. 26: shows the antisense cDNA (SEQ ID NO: 27), which is present in the RNAi construct as shown in FIG. 27;

FIG. 27: shows a diagram of the hairpin construct used for gene silencing of Lewis-type β1,3-galactosyltransferase in A. thaliana plants;

FIG. 28: shows a semi-quantitative RT-PCR of RNA extracted from transgenic plants expressing the gene silencing construct as shown in FIG. 27;

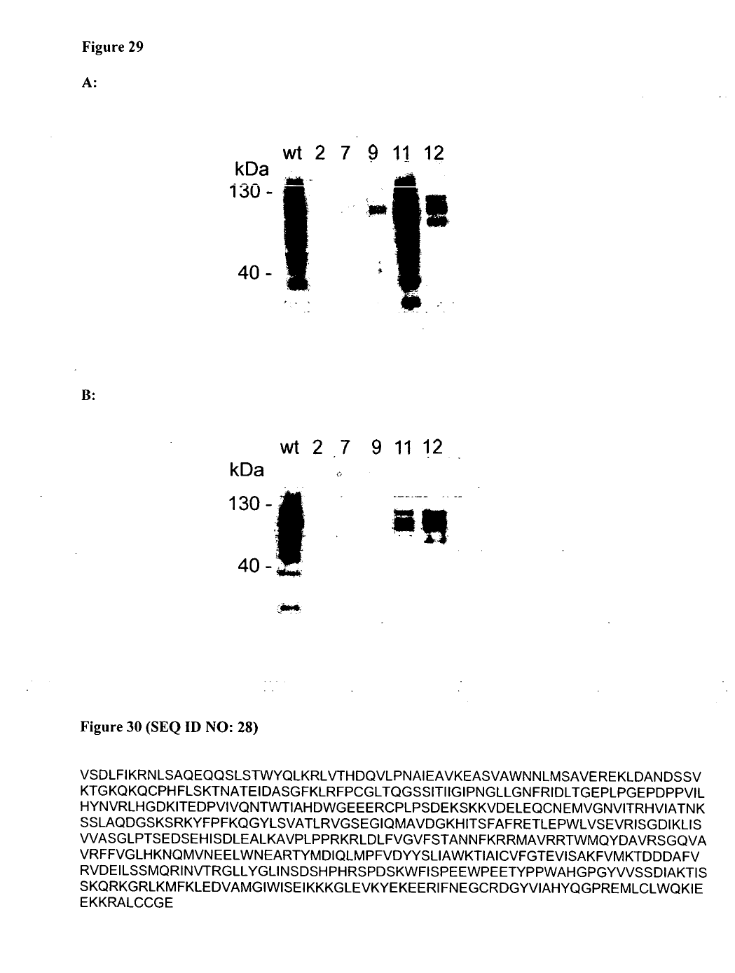

FIG. 29: shows an immunoblot of transgenic plants expressing the gene silencing construct (A): stems, (B): siliques;

FIG. 30: shows the partial amino acid sequence of a putative tobacco (Nicotiana tabacum) Lewis-type β1,3-galactosyltransferase (SEQ ID NO: 28);

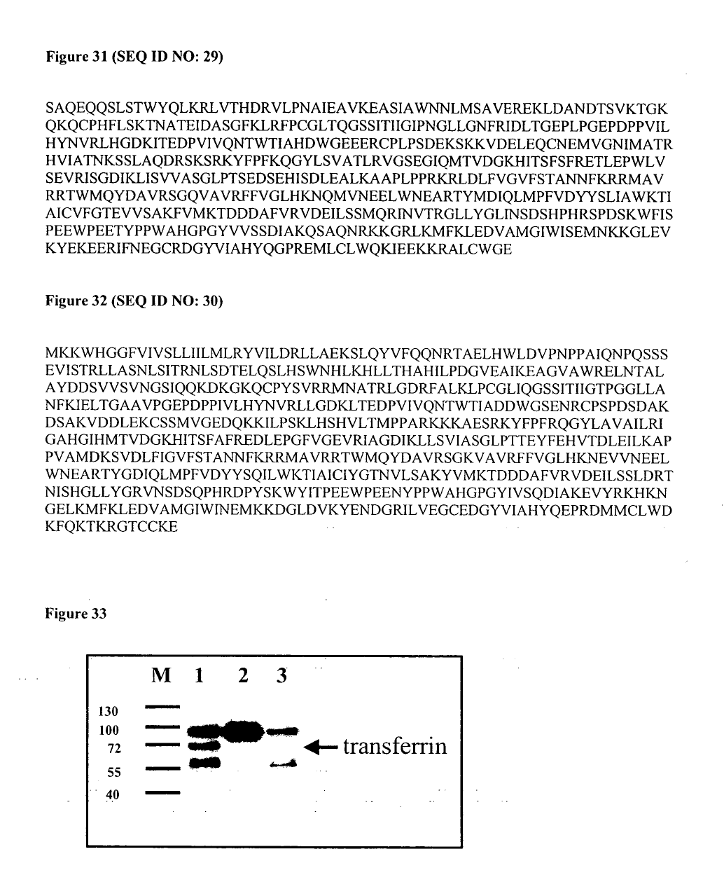

FIG. 31: shows the partial amino acid sequence of a putative Nicotiana benthamiana Lewis-type β1,3-galactosyltransferase (SEQ ID NO: 29);

FIG. 32: shows the amino acid sequences of a putative maize (Zea mays) Lewis-type β1,3-galactosyltransferase (SEQ ID NO: 30);

FIG. 33: shows an immunoblot of human transferrin incubated with the plant Lewis-type β1,3-galactosyltransferase and controls.

### EXAMPLES

The identification of the Lewis-type β1,3-galactosyltransferase from plants was hindered by the fact that the epitope was reported absent from the model plant A. thaliana (Fitchette-Laine et al., 1997, The Plant J., 12: 1411-1417).

In fact, no publication ever described the measurement of the corresponding Lewis-type β1,3-GalT activity in plant cells or extracts from plants.

In addition to that the enzyme, which adds the fucose in α1-4 linkage to the GlcNAc, α1,4-fucosyltransferase from A. thaliana, was expressed in Pichia pastoris, but no activity was detected (see Wilson et al., 2001, Biochimica et Biophysica Acta, 1527: 88-96).

Although recently a publication by Leonard et al., 2002 (Glycobiology 12: 299-306) claimed to have shown the presence of the epitope by Western blots in young A. thaliana seedlings and that the A. thaliana α1,4-fucosyltransferase is an active enzyme, no N-glycan structures carrying Lewis-A epitopes were identified on glycoproteins extracted from A. thaliana so far (no MS-data).

In mammals β1,3-galactosyltransferases occur as a gene family (see Hennet, 2002, Cell. Mol. Life. Sci. 59: 1081-1095; Table I). The detailed function (e.g. in vivo glycosylation of glycoprotein N-glycans) of these enzymes is not completely known (Holgersson and Löfling, 2006, Glycobiology 16: 584-593).

A gene family exists also in A. thaliana. Family GT31 from the CAZY database (http://afmb.cnrs-mrs.fr/CAZY/fam/GT31.html) contains 33 putative galactosyltransferase sequences from A. thaliana. 21 of these sequences contain the PF01762 Galactosyl-T domain and could potentially be candidates for the Lewis-type β1,3-GalT (Table II).

The domain structure of the A. thaliana Lewis type β1,3-GalT is different from the domain structure of mammalian B3GalTs. Mammalian B3GalTs do not contain any Gal-lectin binding domain (PF00337), which is present in the A. thaliana Lewis-type β1,3-GalT (see Tables I and II, appendix 1). Lectin binding domains are very untypical for glycosyltransferases and only found in rare cases (see for example Kubota et al., 2006, J. Mol. Biol. 359: 708-727).

All known mammalian B3GalTs are type II membrane proteins composed of cytoplasmic, transmembrane, stem and catalytic domains as typical for Golgi localised glycosyltransferases. In contrast to that the majority of transmembrane prediction programs predict no transmembrane domain for the A. thaliana Lewis-type β1,3-GalT (AGI code: At1g26810). e.g. Aramemnon database: At1g26810 not predicted as membrane protein http://aramemnon.botanik.uni-koeln.de/seg view.ep?orgm=0&search=At1g26810&cat=0&term=1 Furthermore At1g26810 is not annotated as a transmembrane domain containing protein: http://mips.gsf.de/cqi-bin/proj/thal/search_gene?code=At1q26810 http://www.tigr.org/tigr-scripts/euk_manatee/shared/ORF_infopage.cqi?db=ath1&orf=At1q26810 http://www.plantenergy.uwa.edu.au/applications/suba/flatfile.php?id=AT1G26810.1 http://www.arabidopsis.org/servlets/TairObject?type=locus&name=At1g26810

The A. thaliana Lewis type β1,3-GalT is not included on the available A. thaliana microarrays (e.g. NASCarray. http://affymetrix.arabidopsis.info/; Genevestigator: https://www.genevestigator.ethz.ch/at/), which makes analysis of the function more difficult (e.g. development- or organ-specific expression). Co-expression analysis with the α1,4-fucosyltransferase is not possible (e.g. co-response database: http://csbdb.mpimp-golm.mpg.de/csbdb/dbcor/ath.html#transcor).

TABLE I
Mammalian B3GalTs used to search for A. thaliana orthologues.
The mouse B3GalT1 (MmB3GalT1) was used instead of the
human B3GalT1 (Q9Y5Z6) in all subsequent in silico comparisons,
because it was used by us to generate the Lewis-A epitope in
vivo in A. thaliana leaves (see example 7). Mouse and human B3GalT1
have 100% amino acid sequence identity.

| | | | | | TMD² | | Pfam 01762³ |
|---|---|---|---|---|---|---|---|
| | Swiss-prot | Length (aa) | MW kDa | PI¹ | No. | aa | (aa) |
| MmB3GalT1 | O54904 | 326 | 37.9 | 9.3 | 1 | 7-26 | 92-283 |
| HsB3GalT2 | O43825 | 422 | 49.2 | 9.5 | 1 | 25-43 | 165-359 |
| HsB3GalT5 | Q9Y2C3 | 310 | 36.2 | 8.8 | 1 | 8-28 | 71-261 |

¹Calculated PI DNAstar software (Lasergene)
²Transmembrane domain (TMD) prediction: TMHMM (http://www.cbs.dtu.dk/services/TMHMM/)
³Pfam domain prediction: PF01762: Galactosyltransferase (http://www.sanger.ac.uk/Software/Pfam/)

TABLE II
21 A. thaliana sequences present in CAZY family GT31,
which contain the GalT Pfam domain. The members of subfamily 1
(see FIG. 1) are shown underlined, and the Lewis-type β1,3-GalT
is shown in bold.

| | | | | | | | | Pfam | Pfam |
|---|---|---|---|---|---|---|---|---|---|
| | GenBank | Swissprot | Length | MW | | TMD² | | 01762³ | 00337⁴ |
| AGI code⁵ | Accession | Accession | (aa) | kDa | PI¹ | No. | aa | (aa) | (aa) |
| At1g05170 | NM_100395 | | 404 | 45.6 | 9.0 | 1 | 13-35 | 149-347 | — |
| At1g11730 | NM_101045 | | 384 | 43.6 | 9.1 | 1 | 21-43 | 129-327 | — |
| At1g22015 | NM_102051 | | 398 | 45.2 | 8.3 | 1 | 13-32 | 140-338 | — |
| At1g26810 | NM_102445 | Q8L7F9 | 643 | 72.3 | 5.0 | 0 | — | 406-597 | 170-363 |
| At1g27120 | NM_102474 | Q8GXG6 | 673 | 77.0 | 7.3 | 1 | 13-32 | 439-633 | 184-393 |
| At1g32930 | NM_103026 | | 399 | 44.6 | 7.2 | 1 | 13-35 | 144-342 | — |
| At1g33430 | NM_103068 | | 395 | 44.7 | 8.8 | 1 | 5-27 | 138-336 | — |
| At1g53290 | NM_104207 | | 346 | 38.7 | 9.0 | 1 | 21-43 | 100-295 | — |
| At1g74800 | NM_106138 | Q8RX55 | 672 | 77.3 | 8.6 | 1 | 27-49 | 437-625 | 190-391 |
| At1g77810 | NM_106430 | | 393 | 44.6 | 7.1 | 1 | 9-31 | 132-336 | — |
| At2g25300 | NM_128087 | | 341 | 39.1 | 8.3 | 0 | — | 133-330 | — |
| At2g26100 | NM_128168 | | 371 | 41.2 | 8.4 | 1 | 46-68 | 76-319 | — |
| At2g32430 | NM_128802 | | 409 | 46.5 | 9.1 | 1 | 20-42 | 154-352 | — |
| At3g06440 | NM_111519 | Q9ASW1 | 619 | 70.6 | 7.1 | 0 | — | 385-580 | 165-343 |
| At3g14960 | NM_112358 | | 343 | 38.7 | 8.0 | 1 | 21-43 | 98-293 | — |
| At4g21060 | NM_118224 | Q9SUA8 | 741 | 84.3 | 8.5 | 1 | 70-92 | 507-696 | 247-461 |
| At4g26940 | NM_118828 | | 407 | 46.0 | 7.3 | 0 | — | 153-351 | — |
| At4g32120 | NM_119364 | | 345 | 39.3 | 8.5 | 0 | — | 132-329 | — |
| At5g53340 | NM_124713 | | 338 | 37.8 | 8.4 | 1 | 13-32 | 124-322 | — |
| At5g57500 | NM_125131 | | 318 | 37.0 | 7.5 | 1 | 12-29 | 83-239 | — |
| At5g62620 | NM_125657 | Q9LV16 | 681 | 77.7 | 8.6 | 1 | 26-48 | 446-637 | 186-400 |

¹Calculated PI (http://www.arabidopsis.org/)
²Transmembrane domain (TMD) prediction: TMHMM (http://www.cbs.dtu.dk/services/TMHMM/)
³Pfam domain prediction: PF01762: Galactosyltransferase (http://www.sanger.ac.uk/Software/Pfam/)
⁴Pfam domain prediction: PF00337: Gal-binding-lectin (http://www.sanger.ac.uk/Software/Pfam/)
⁵Unique gene id: http://mips.gsf.de/proj/thal/db/about/codes.html

A comparison of Table I and II reveals that the A. thaliana Lewis-type β1,3-GalT (At1g26810) is different from the mammalian B3GalTs with respect to length of the protein, PI, number of TMDs and domain structure. There are many other A. thaliana proteins which are based on the listed parameters better candidates (e.g. At1g05170, At1g11730, At1g22015, At1g33430, At1g53290, . . . ).

TABLE III
Amino acid sequence identities among B3GalT1-like
proteins:
The full-length coding sequences (Met-Stop) were used in
pair-wise BLAST alignments (Matrix: Blossum62) to obtain the
identity (similarity) values in %
(http://www.ncbi.nlm.nih.gov/blast/bl2seq/).

| | | | | | | Hs | Hs | Mm |
|---|---|---|---|---|---|---|---|---|
| | At1g27120 | At1g74800 | At3g06440 | At4g21060 | At5g62620 | B3GalT5 | B3GalT2 | B3GalT1 |
| At1g26810 | 34 (52) | 33 (50) | 43 (59) | 32 (48) | 35 (50) | 28 (46) | 25 (43) | 28 (46) |
| At1g27120 | — | 62 (75) | 34 (51) | 51 (66) | 58 (70) | 25 (44) | 23 (41) | 28 (45) |
| At1g74800 | | — | 34 (52) | 55 (72) | 66 (76) | 22 (41) | 25 (44) | 31 (51) |
| At3g06440 | | | — | 33 (50) | 34 (51) | 29 (49) | 27 (46) | 33 (54) |
| At4g21060 | | | | — | 54 (69) | 27 (45) | 21 (44) | 29 (55) |
| At5g62620 | | | | | — | 27 (45) | 23 (43) | 33 (52) |
| HsB3GalT5 | | | | | | — | 41 (61) | 43 (60) |
| HsB3GalT2 | | | | | | | — | 52 (69) |
| MmB3GalT1 | | | | | | | | — |

TABLE IV
Amino acid sequence identities among B3GalT1-like proteins:
Only the GalactosylT-domain sequences (pfam01762) of the A. thaliana
candidate proteins (see Table II, subfamily 1) and pfam
domains of the mammalian B3GalTs (see Table I) were used in
pair-wise BLAST alignments to obtain the identity (similarity)
values in % (http://www.ncbi.nlm.nih.gov/blast/bl2seq/).

| | | | | | | Hs | Hs | Mm |
|---|---|---|---|---|---|---|---|---|
| | At1g27120 | At1g74800 | At3g06440 | At4g21060 | At5g62620 | B3GalT5 | B3GalT2 | B3GalT1 |
| At1g26810 | 40 (61) | 40 (59) | 57 (72) | 39 (59) | 44 (61) | 33 (52) | 30 (48) | 31 (54) |
| At1g27120 | — | 70 (80) | 43 (64) | 62 (79) | 71 (84) | 30 (53) | 27 (48) | 32 (52) |
| At1g74800 | | — | 40 (62) | 60 (75) | 73 (84) | 27 (49) | 27 (46) | 31 (51) |
| At3g06440 | | | — | 41 (64) | 45 (67) | 33 (55) | 29 (50) | 32 (54) |
| At4g21060 | | | | — | 63 (83) | 28 (50) | 23 (45) | 29 (55) |
| At5g62620 | | | | | — | 31 (52) | 25 (48) | 30 (52) |
| HsB3GalT5 | | | | | | — | 48 (67) | 51 (65) |
| HsB3GalT2 | | | | | | | — | 58 (74) |
| MmB3GalT1 | | | | | | | | — |

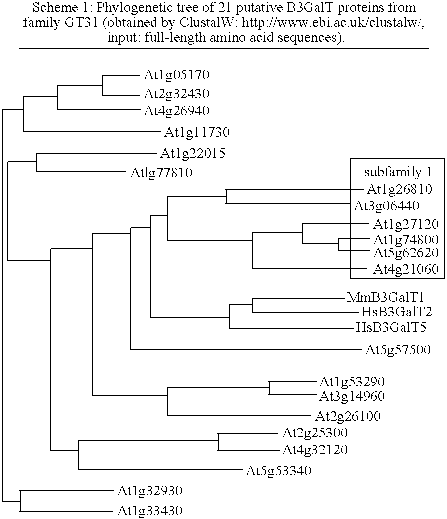

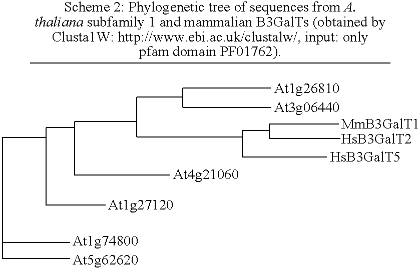

As shown in Scheme 1 A. thaliana contains a subfamily (subfamily 1) consisting of six sequences, which display homology to the mammalian B3GalTs. An alignment of the putative galactosyltransferase domain reveals that two sequences (At1g26810 and At3g06440) are closest to the mammalian B3GalTs based on the analysis of the primary amino acid sequence. Interestingly, for both A. thaliana proteins no transmembrane domain is predicted. All six members of subfamily 1 contain in addition to the GalT domain a Gal-lectin binding domain (PF00337).

A pairwise BLAST comparison (Tables III and IV) and ClustalW analysis (Scheme 2) shows that the members of A. thaliana subfamily 1 share a similar degree of identity to the mammalian B3-GalTs (e.g. 29-32% identity to MmB3GalT1 (Table IV). From these data it cannot be deduced that the At1g26810 protein is the obvious A. thaliana Lewis-type β1,3-GalT. The comparison of conserved motifs reveals also no obvious A. thaliana Lewis-type β1,3-GalT protein (Table V).

A BLASTP search in the A. thaliana database (AGI proteins; http://www.arabidopsis.org/Blast/) with the MmB3GalT1 or HsB3-GalT2 sequences as query lists At1g26810 as the first hit. A search using HsB3GalT5 lists At3g06440 as the first hit and At1g26810 as the second hit. These searches reveal that the E-values of the first six hits (=all six members of A. thaliana subfamily 1) are in a similar low range, which makes all six of them good candidates for a Lewis-type β1,3-GalT acting on N-glycan substrates (highly similar results were obtained by FASTA and WU-BLAST2 searches, http://www.arabidopsis.org/).

Scheme 3: Alignment of sequences from A. thaliana subfamily 1 with
mammalian B3GalTs. (obtained by ClustalW: http://www.e-
bi.ac.uk/clustalw/, input: only pfam domain PF01762).

CLUSTAL W (1.83) multiple sequence alignment

| | | |
|---|---|---|
| At1g26810 | KRRMAVRRTWMQYDDVRSGRVAVRFFVGLHKSP--LVNLELWNEARTYGDVQLMPFVDYY | 58 |
| At3g06440 | KRRMALRRSWMQYEAVRSGKVAVRFLIGLHTNE--KVNLEMWRESKAYGDIQFNPFVDYY | 58 |
| At1g74800 | SERMAVRKSWMQHVLITSAKVVARFFVALHGRK--EVNVELKKEAEYFGDIVLVPYMDSY | 58 |
| At5g62620 | AERMAVRRSWMQHKLVKSSKVVARFFVALHSRK--EVNVELKKEAEFFGDIVIVPYMDSY | 58 |
| At1g27210 | AERMAVRKSWMQQKLVRSSKVVARFFVALHARK--EVNVDLKKEAEYFGDIVIVPYMDHY | 58 |
| At4g21060 | SERMAVRKTWMQHPSIKSSDVVARFFVALNPRK--EVNAMLKKEAEYFGDIVILPRMDRY | 58 |
| MmB3GalT1 | DARQAIRETWGDENNFKGIKIATLFLLGKNADP--VLNQMVEQESQIFHDIIVEDFIDSY | 58 |
| HsB3GalT2 | EARRAIRQTWGNESLAPGIQITRIFLLGLSIKLNGYLQRAILEESRQYHDIIQQEYLDTY | 60 |
| HsB3GalT5 | AERMAIRQTWGKERMVKGKQLKTFFLLGTTSSA--AETKEVDQESQRHGDIIQKDFLDVY | 58 |
| | * *:*.:* . . : *::. : .*:. . *: ::* * | |
| At1g26810 | SLILWKTLA-ICIFGTEVDSAKFIMKTDDDAFVRVDEVLLSLSM--TMNTRGLIYGLINS | 115 |
| At3g06440 | GLLSLKTVA-LCILGTKVIPAKYIMKTDDDAFVRIDELLSSLEE--RPSS-ALLYGLISF | 114 |
| At1g74800 | DLVVLKTVA-ICEHGALAFSAKYIMKCDDDTFVKLGAVINEVKK--VPEGRSLYIGNMNY | 115 |
| At5g62620 | DLVVLKTVA-ICEYGAHQLAAKFIMKCDDDTFVQVDAVLSEAKK--TPTDRSLYIGNINY | 115 |
| At1g27120 | DLVVLKTVA-ICEYGVNTVAAKYVMKCDDDTFVRVDAVIQEAEK--VKGRESLYIGNINF | 115 |
| At4g21060 | ELVVLKTIA-ICEFGVQNVTAPYIMKCDDDTFIRVESILKQIDG--VSPEKSLYMGNLNL | 115 |
| MmB3GalT1 | HNLTLKTLMGMRWVATFCSKAKYVMKTDSDIFVNMDNLIYKLLKPSTKPRRRYFTGYVIN | 118 |
| HsB3GalT2 | YNLTIKTLMGNWWVATYCPHIPYVMKTDSDMFVNTEYLINKLLKPDLPPRHNYFTGYLMR | 120 |
| HsB3GalT5 | YNLTLKTMMGIEWVHRFCPQAAFVMKTDSDMFINVDYLTELLLKKNRTTR--FFTGFLKL | 116 |
| | : **: : ::** *.* *:. : * : | |
| At1g26810 | DSQPIRNPDSKWYISYEEWPEEKYPPWAHGPGYIVSRDIAESVGKLFKEGNLKMFKLEDV | 175 |
| At3g06440 | DSSPDREQGSKWFIPKEEWPLDSYPPWAHGPGYIISHDIAKFVVKGHRQRDLGLFKLEDV | 174 |
| At1g74800 | YHKPLRG--GKWAVTYEEWPEEDYPPYANGPGYVLSSDIARFIVDKFERHKLRLFKMEDV | 173 |
| At5g62620 | YHKPLRQ--GKWSVTYEEWPEEDYPPYANGPGYILSNDISRFIVKEFEKHKLRMFKMEDV | 173 |
| At1g27120 | NHKPLRT--GKWAVTFEEWPEEYYPPYANGPGYILSYDVAKFIVDDFEQKRLRLFKMEDV | 173 |
| At4g21060 | RHRPLRT--GKWTVTWEEWPEAVYPPYANGPGYIISSNIAKYIVSQNSRHKLRLFKMEDV | 173 |
| MmB3GalT1 | G-GPIRDVRSKWYMPRDLYPDSNYPPFCSGTGYIFSADVAELIYK--TSLHTRLLHLEDV | 175 |
| HsB3GalT2 | GYAPNRNKDSKWYNPPDLYPSERYPVFCSGTGYVFSGDLAEKIFK--VSLGIRRLHLEDV | 178 |
| HsB3GalT5 | NEFPRIQPFSKWFVSKSEYPWDRYPPFCSGTGYVFSGDVASQVYN--VSKSVPYIKLEDV | 174 |
| | * * .** :. . :* ** :. *.**:.* ::: : . :::*** | |
| At1g26810 | AMGIWIAELTKHGLEPH------ | 192 |
| At3g06440 | AMGIWIQQFNQTIKRVKYINDK- | 196 |
| At1g74800 | SVGMWVEHFKNTTNPV------- | 189 |
| At5g62620 | SVGMWVEQFNNGTKPVDYI---- | 192 |
| At1g27120 | SMGMWVEKFN-ETRPVAVVHSLK | 195 |
| At4g21060 | SMGLWVEQFNASMQPVE------ | 190 |
| MmB3GalT1 | YVGLCLRKLGIHPFQNX------ | 192 |
| HsB3GalT2 | YVGICLAKLRIDPVPPP------ | 195 |
| HsB3GalT5 | FVGLCLERLNIRLEELH------ | 191 |
| | :*: : .: | |

Comparison A. thaliana and Rice Sequences

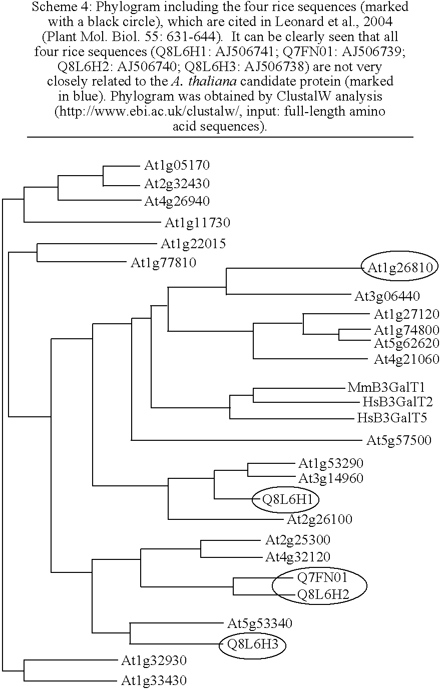

### These 4 Cited Rice Sequences Could be Excluded as Lewis-Type β1,3-GalTs:

Other rice sequences (like Q67X5J and Q69JV6, see Scheme 5 and Table V) display a higher sequence identity to the A. thaliana Lewis-type β1,3-GalT protein.

The domain structure of the four proteins is different from the A. thaliana Lewis-type β1,3-GalT protein. They do not contain any lectin binding domain (PF00337).

Overexpression of A. thaliana protein At1g53290, which seems to be an orthologue of rice protein Q8L6H1 in A. thaliana leaves, showed no increase in Lewis-A epitope expression.

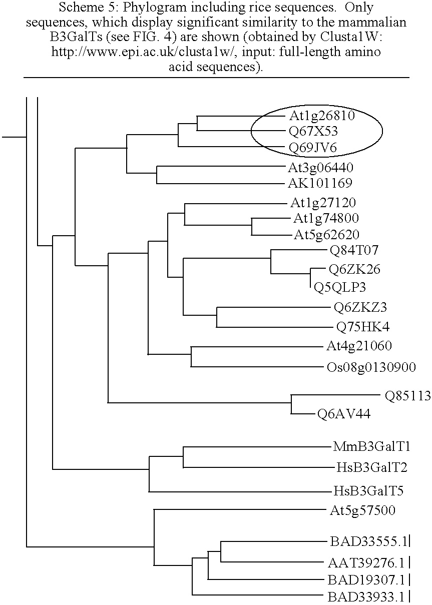

TABLE V
Rice Lewis-type β1,3-GalT candidate proteins:

| Unirprot | Genbank | Length | MW | | PF01762 | PF00337 | | |
|---|---|---|---|---|---|---|---|---|
| ID | Acc. Nr. | aa | kDa | PI | (aa-aa) | (aa-aa) | % identity¹ | % similarity¹ |
| Q69JV6 | BAD34256 | 621 | 69.0 | 6.22 | 386-582 | 157-343 | 72 | 84 |
| Q67X53 | BAD37266 | 637 | 72.0 | 6.43 | 401-597 | 163-358 | 76 | 86 |

¹The identity and similarity values are obtained by pairwise BLAST alignment of the PF01762 galactosyltransferase domains with the PF01762 domain of At1g26810. http://www.ncbi.nlm.nih.gov/blast/bl2seq/wblast2.cgi)

Q69V6 and Q67X53 PF01762 domains display 76% identity (87% similarity) to each other.

Based on the sequence identity and predicted domain structure the rice genome could contain two sequences, which represent functional Lewis-type β1,3-GalTs.

Comparison of A. thaliana and Sequences from Other Species

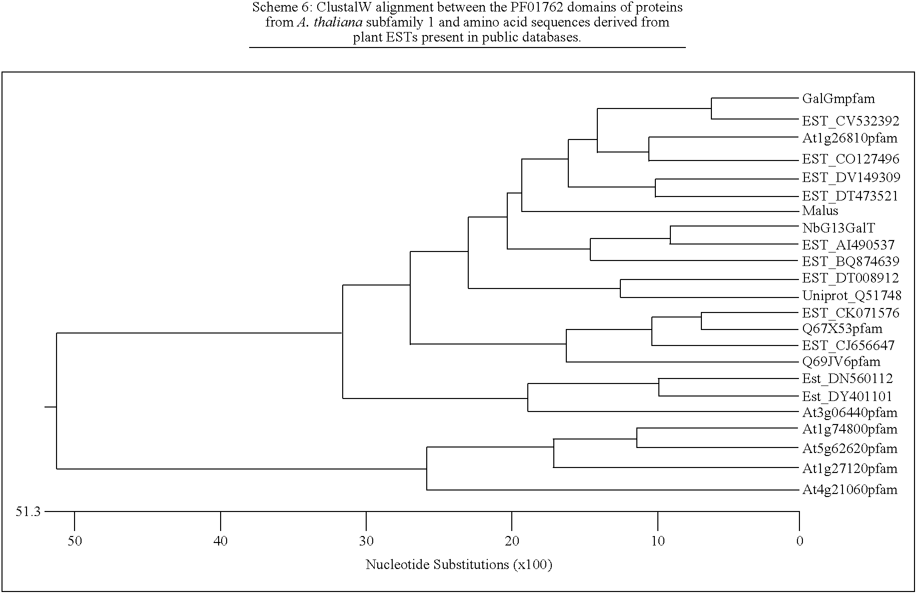

Sequences with More than 75% Identity (to At1q26810) in the PF01762 Domain:

| | species |
|---|---|
| Name(sequences are listed | |
| in appendix 2) | |
| GalGm | Glycine max (soybean) sequence assembled |
| | from several ESTs |
| Malus | Malus domestica (apple) assembled from |
| | several ESTs |
| Nb13GalT | Nicotiana benthamiana partial cDNA |
| Genbank accession | |
| CO127496 | Gossypium raimondii (cotton) EST |

EST Sequences with More than 70% Identity to At1q26810 (Full-Length Amino Acid Sequence):

| Genbank accession | species |
|---|---|
| CV532292 | Phaseolus vulgaris (common bean) EST |
| DV149309 | Euphorbia esula (leafy spurge) EST |
| DT473521 | Populus trichocarpa (poplar) EST |
| AI490537 | Lycopersicon esculentum (tomato) EST |
| BQ874639 | Lactuca sativa (lettuce) EST |
| DT008912 | Vitis vinifera (grapewine) EST |
| Q5I748 | Fortunella hindsii swissprot (kumquat) par- |
| | tial cDNA |
| CK071576 | Oryza sativa indica cultivar-group (rice) partial |
| | cDNA |
| CJ656647 | Triticum aestivum (wheat) partial cDNA |

An important part of the invention was therefore the surprising results showing unambiguously the presence of N-linked glycans with Lewis-A structures in A. thaliana by immunoblots and mass spectroscopy. This result implied the presence of a Lewis-type β1,3-galactosyltransferase in A. thaliana and enabled to perform a search in the A. thaliana genome for candidate genes. Based on homology searches using mammalian β1,3-galactosyltransferases (Hennet, 2002, Cell. Mol. Life. Sci. 59: 1081-1095) it was possible to identify 21 putative candidate genes. The biological function of all of these genes was completely unknown. To identify the plant Lewis-type β1,3-galactosyltransferase the mRNA expression level of the 21 candidate genes was analysed and correlated with the expression pattern of the Lewis-A epitope in different organs of the plants. Based on the data derived from homology searches and mRNA expression transgenic A. thaliana plants were generated, which overexpressed the candidate genes in tissues normally lacking the Lewis-A epitope. Screening of the transgenic overexpression lines resulted in the identification of the complete open reading frame (SEQ ID NO: 1) coding for the Lewis-type β1,3-galactosyltransferase (SEQ ID NO: 2) from A. thaliana. The β1,3-galactosyltransferase activity was also shown by in vitro assays using heterologously expressed recombinant plant Lewis-type β1,3-galactosyltransferase. In the databases SEQ ID NO: 1 was annotated as coding for an “unknown protein” (GenBank Acc. Nr. AAM91658) and as “galactosyltransferase family protein” (GenBank Acc. Nr.: NP_{—}174003), but was neither annotated as β1,3-galactosyltransferase, nor as Lewis-type galactosyltransferase, nor as Lewis type β1,3-galactosyltransferase.

In the rice genome database (http://rgp.dna.affrc.go.jp/) two rice amino acid sequences (GenBank Acc. Nr. BAD37266 and BAD34256), which have more than 50% identity to SEQ ID NO: 2 (based on ClustalW analysis), were classified as “putative beta-1,3-galactosyltransferase”. This classification is based on homology search only and is not verified by experimental data. None of the two sequences was classified as a Lewis-type β1,3-galactosyltransferase. Furthermore the A. thaliana as well as the rice databases contain other sequences, which have been annotated as either “putative beta-1,3-galactosyltransferase” (e.g. GenBank Acc. Nrs. AAQ77230, AAQ77230) or “beta1,3-galactosyltransferase-like protein” (e.g. GenBank Acc. Nrs. CAD44836, CAD44837, CAD44838, CAD44839). All of these sequences have in common that the sequence identity to SEQ ID NO: 2 is below 50%. None of these sequences have been annotated as a Lewis-type β1,3-galactosyltransferase and no experimental data are shown which describe a corresponding activity.

### Example 1

### RT-PCR and cDNA Cloning of Lewis-Type β1,3-Galactosyltransferase

The entire RNA was isolated from siliques of A. thaliana ecotype Columbia using the SV Total RNA isolation kit (Promega). RNA was treated with DNase to remove traces of genomic DNA. First-strand cDNA was synthesized from 500 ng of total RNA at 42° C. using oligo(dT) primers and AMV reverse transcriptase (Promega).

The first strand cDNA was subjected to a PCR, wherein a forward primer (SEQ ID NO: 5; FIG. 16), a reverse primer (SEQ ID NO: 6; FIG. 17) and a pfu-Polymerase (Stratagene) containing a proofreading activity were used. The primers for amplification contained KpnI and BamHI restriction enzyme cleavage sites to facilitate subsequent cloning steps. The amplified PCR product was a DNA fragment comprising the putative open reading frame and additional base pairs for the cleavage sites on each end (FIG. 10, lane 2). Subsequently the PCR product was purified from an agarose-gel using a gel extraction kit (Promega), subcloned into a Zero Blunt Topo-cloning vector (Invitrogen) and sequenced by means of the didesoxynucleotide method (BIG Dye termination cycle sequencing kit, Applied Biosystems). The sequence of the amplified open reading frame has a size of 1932 bp (SEQ ID NO: 1) and codes for a protein of 643 amino acids (SEQ ID NO: 2) having a calculated molecular weight of 72.3 kDa and a theoretical pI value of 5.0. Apart from a base pair change (at position 1206: “G” instead of “A”) SEQ ID NO: 1 is identical to the annotated genomic sequence (SEQ ID NO: 3) after removing of 6 introns. The nucleotide change from G to A does not lead to a change of the respective amino acid (GCA and GCG code for alanine).

Comparison of SEQ ID NO: 2 with other amino acid sequences from A. thaliana reveals no protein with identity of more than 50%. Comparison of SEQ ID NO: 2 with sequences from rice reveals two proteins with more than 50% identity. A BLAST search in databases accessible via NCBI (http://www.ncbi.nlm.nih.gov/BLAST/) or present in the Plant Genome DataBase (http://www.plantgdb.org/) showed sequences homologous to SEQ ID NO: 2 in other plant species (e.g. tomato: AI490537; onion: CF436881; soybean: BM085070; potato: BG599392; tobacco: EB682269; cotton: C0127496; bean: CV532292; lettuce: BQ874639; poplar: DT473521; wheat: CJ656647). An alignment of amino acid sequences homologous to SEQ ID NO: 4 is shown in FIG. 8. The alignment was done using ClustalW (http://www.ebi.ac.uk/clustalw/) and residues, which are identical to SEQ ID NO: 4 are boxed and shaded. For each protein the aligned sequence stretch corresponds to the putative “galactosyltransferase domain” (PF01762) as predicted by the Pfam protein family database (http://www.sanger.ac.uk/Software/Pfam/Sanger; Bateman et al., 2004, Nucleic Acids Res., Database Issue 32: D138-D141; see also FIG. 9).

### Example 2

### Expression of Recombinant Plant Lewis-Type β1,3-Galactosyltransferase in Leaves of A. thaliana Plants

The entire coding region for the assumed Lewis-type β1,3-galactosyltransferase (SEQ ID NO: 1) was removed from the Topo-cloning vector by KpnI and BamHI digestion and subcloned into KpnI/BamHI digested and dephosphorylated plant expression vector pPT2 (Strasser et al., 2005, Biochem. J. 387: 385-391). Correct cloning was confirmed by sequencing. In the vector pPT2, the recombinant Lewis-type β1,3-galactosyltransferase protein will be expressed from the strong constitutive cauliflower mosaic virus 35S promoter. The Agrobacterium tumefaciens strain UIA143 containing the pMP90 plasmid (Hamilton, 1997, Gene, 200: 107-116) was used to transform A. thaliana ecotype Columbia plants by the floral dip procedure (Clough and Bent, 1998, The Plant J. 16: 735-743). Seeds were selected on MS-medium (Sigma) (Murashige and Skoog, 1962, Physiol. Plant 15: 473-497) containing 100 mg/l kanamycin and subsequently transferred to soil. Genomic DNA was isolated by following the procedure of the GenElute Plant Genomic DNA Purification Kit (Sigma).

The integration of the heterologous SEQ ID NO: 1 sequence was confirmed by PCR from genomic DNA using forward primer 5′-AAGGCCACTGGATCTCGTTATTG-3′ (SEQ ID NO: 13) and reverse primer 5′-TTACCAACCGATTCTGCTATGTCAC-3′ (SEQ ID NO: 14), which amplify a 528 bp fragment from SEQ ID NO: 1 (FIG. 11, panel A). This PCR-fragment is derived from the integrated transgene since a PCR-product from the endogenous gene SEQ ID NO: 3 results in a larger 788 bp product due to the presence of an intron. To confirm the expression of the integrated transgene corresponding to SEQ ID NO: 1 RNA was isolated from leaves of transgenic plants using the SV Total RNA isolation kit (Promega). Total RNA was treated with DNAse (Promega) to remove any traces of genomic DNA. cDNA was made from 500 ng of total RNA using oligo(dT) primers and AMV-reverse transcriptase (Promega) in a total volume of 20 μl. To analyse the quality of the cDNA a control reaction was performed using forward primer 5′-GGTGCTCGGAATTTTCGTCTACA-3′ (SEQ ID NO: 15) and reverse primer 5′-TCATTCTCTTTGCAACTGGCTCTA-3′ (SEQ ID NO: 16), which amplify a 319 bp fragment of the α1,3-fucosyltransferase B cDNA (FIG. 11, panel B). This fragment is detectable in A. thaliana wildtype plants and in transgenic lines containing the stable integration of the SEQ ID NO: 1 T-DNA construct. To detect the expression of the transcript derived from the transgene in the transgenic plants the forward gene-specific primer SEQ ID NO: 13 and a reverse pPT2 vector-specific primer 5′-ATTGCCGTAGATGAAAGACTGA-3′ (SEQ ID NO: 17) (FIG. 11, panel C) were used for PCR. As a result a fragment of the expected size of 847 bp was detected in all trans-genic lines. In the wildtype control plant, which is not trans-genic, no corresponding transcript could be detected. In addition to the amplification of the cDNA with primers SEQ ID NO: 13 and SEQ ID NO: 14 resulted in the detection of increased amounts of product (SEQ ID NO: 1 expression) compared to wildtype plants (FIG. 11, panel D).

In order to detect changes in the proportion of glycoproteins carrying Lewis-A epitopes leaves from transgenic plants and non-transgenic plants (wildtype plants ecotype Columbia) were analysed by immunoblot and the intensity of the signal was compared. Leaves of A. thaliana wildtype plants are very much suitable for that screening approach since they only contain trace amounts of proteins having Lewis-A epitopes (Fitchette-Laine et al., 1997, The Plant J. 12: 1411-1417; FIG. 12). Leaves were ground in liquid nitrogen and proteins were extracted in extraction buffer containing 10 mM Tris pH 8.0, 1 mM EDTA and 1% (w/v) SDS.

Plant material was ground in liquid nitrogen, resuspended in phosphate-buffered saline buffer (137 mM NaCl, 2.7 mM KCl, 10 mM Na2HPO4, 2 mM KH2PO4, pH 7.4) supplemented with 1% Triton X100 (10 μl per mg of plant material) and cleared by centrifugation (two times 3 min at 16 000 g). The protein content in the samples was determined using the BCA protein assay kit (Pierce) and bovine serum albumin as a standard.

10 μg of each sample were mixed with SDS-polyacrylamide gel electrophoresis (PAGE) loading buffer, denatured at 95° C. for 5 min and subjected to 12.5% SDS-PAGE under reducing conditions. Separated proteins were blotted onto Hybond enhanced chemiluminescence (ECL) nitrocellulose membranes (Amersham Biosciences). The blot was blocked in 3% (w/v) bovine serum albumin in phosphate-buffered saline buffer for 1 h and incubated in a 1:40 dilution of the rat monoclonal JIM84 antibody (Horsley et al., 1993, Journal of Exp. Bot. 44, Supplement: 223-229) in phosphate-buffered saline buffer supplemented with 0.1% (v/v) Tween 20. In plants the JIM84 antibody recognizes specifically the Lewis-A epitope on glycoproteins (Fitchette et al., 1999, Plant Physiol. 121: 333-343). The detection was performed after incubation in a 1:20000 dilution of a horseradish peroxidase-conjugated goat anti-rat IgM antibody (Jackson) in phosphate-buffered saline buffer with 0.1% (v/v) Tween 20 with Supersignal West Pico chemiluminescent substrate (Pierce). As shown in FIG. 12 a clear signal is detected in transgenic plants (#12-#23), which overexpress the plant Lewis-type β1,3-galactosyltransferase in A. thaliana leaves. In contrast to that the control (Col-0) does not show any Lewis-A epitope.

To confirm the increase of the Lewis-A epitope containing N-glycans in the transgenic plants plant material (leaves, stems, siliques) was subjected to total N-glycan analyses performed according to Kolarich and Altmann, 2000 (Anal. Biochem. 285: 64-75). FIG. 13 shows mass spectra of N-glycans isolated from wildtype leaves compared to leaves from transgenic plants. The presence of galacotsylated peaks in the transgenic plant is marked by arrows.

### Example 3

### Expression of Recombinant Plant Lewis-Type β1,3-Galactosyltransferase in Insect Cells

The coding region of the assumed plant Lewis-type β1,3-galactosyltransferase lacking the putative cytoplasmatic and transmembrane region was amplified by PCR from the Topo-cloning vector using the forward primer 5′-TATACTGCAGAAGCCTTATATTACTGCTGCT-3′ (SEQ ID NO: 11) and the reverse primer 5′-TAAAGGTACCTTACCATTCGCGGCAGCAAAGAG-3′ (SEQ ID NO: 12), digested with PstI and KpnI and cloned into PstI/KpnI digested and dephosphorylated baculovirus transfer vector pVTBacHis1 (Sarkar et al., 1998; Glycoconj. J. 15: 193-197). After the final cloning step, the expression construct was subjected to DNA sequencing to rule out any artifactual mutation. DNA sequencing was performed in a thermocycler using the BigDye Terminator 3.1 Cycle Sequencing kit and a Prism 3100 genetic analyzer (Applied Biosystems). Sequence data were obtained using gene-specific or vector derived sense and antisense oligonucleotides as the primers.

Spodoptera frugiperda Sf9 and Sf21 cells (both obtained from A.T.C.C., Manassas, Va., U.S.A.) were grown in IPL-41 (Sigma, St. Louis, Mo., U.S.A.) medium containing 5% (v/v) heat inactivated fetal bovine serum (Invitrogen). Each recombinant baculovirus transfer vector (1 μg) was co-transfected with 200 ng of Baculo-Gold viral DNA (BD PharMingen, Erembodegem, Belgium) into Sf9 cells using Lipofectin (Invitrogen) according to the manufacturer's instructions. After maintaining for 5 days at 27° C., the supernatant containing recombinant baculovirus was used for the infection of Sf21 cells, which were cultivated in IPL-41 medium containing 5% (v/v) heat inactivated fetal bovine serum. After 4 days at 27° C. cells and conditioned media were harvested. Cells were washed 2× with phosphate-buffered saline solution and resuspended in the following buffer (1 ml per 10⁷ cells): 100 mM MES buffer, pH 7.0, with 1% Triton X-100, 1 mM DTT, 1 mM PMSF, 5 μg/ml Leupeptin (Sigma), 5 μg/ml E-64 (Serva) and incubated on ice for 30 min.

### Example 4

### Assay for the Plant Lewis-Type β1,3-Galactosyltransferase

Recombinant expressed plant Lewis-type β1,3-galactosyltransferase was purified from the medium of Sf9 insect cells infected with recombinant virus harboring the plant enzyme using a column of chelating Sepharose (Amersham Biosciences) charged with Ni2+ ions (according to Bencur et al., 2005, Biochem. J. 388: 515-525). Purified plant Lewis-type β1,3-galactosyltransferase protein was assayed using a dabsylated glycopeptide substrate and analyzed by MALDI-TOF MS (as described in Strasser et al., 2000, FEBS LETT. 472: 105-108). Briefly, the assay mixture contained, in a total volume of 20 μl, 2 μg of purified protein, 2 nmol dabsylated GnGn-peptide as acceptor substrate (FIG. 14), 1 mM UDP-galactose as donor substrate, 10 mM ATP, 20 mM MnCl2 and 1 mM 2-acetamido-1,2-dideoxy-nojirimycin was included to prevent degradation of the product by N-acetylglucosaminidase. The samples were incubated for 3 and 16 hours at 30° C. and analyzed by MALDI-TOF mass spectrometry. Protein purified from mock infected insect cells was analyzed as a negative control.

### Example 5

### Mass Spectrometry of the Plant Lewis-Type β1,3-Galactosyltransferase Product

Mass spectrometry was performed on a DYNAMO (BioAnalysis, Santa Fe, N. Mex.), a MALDI-TOF MS, which is capable of dynamic extraction (synonym for late extraction). After addition of 2% 2,5-dihydroxy benzoic acid, the samples containing dabsylated glycopeptides were immediately dried by applying vacuum.

FIG. 15A shows the measured enzyme activity of the recombinant Lewis-type β1,3-galactosyltransferase as well as of the negative control, when dabsylated GnGn-peptide was used as a substrate. The detected increase in mass (162 Da for a single galactose residue) corresponds well with the expected value, which confirms the cloning of an active galactosyltransferase. To rule out that the galactose was added in β1,4-linkage to the glycopeptide, the galactosylated GnGn-peptide was incubated overnight with Aspergillus oryzae β-galactosidase, which was shown to remove β1,4-linked and β1,6-linked galactose (Zeleny et al., 1997, Anal. Biochem. 246: 96-101). As shown in FIG. 15B the galactosylated peak is resistant to Aspergillus oryzae β-galactosidase treatment. In contrast to that, treatment with bovine testes galactosidase, which removes both β1,3- and β1,4-linked, yielded again a GnGn-peptide and incubation with β1,3-galactosidase (New England Biolabs) resulted in the removal of the terminal galactose residues. These data show the presence of terminal β1,3-linked galactose.

### Example 6

### HPLC-Analysis of the Plant Lewis-Type β1,3-Galactosyltransferase Product

Galactosyltransferase assays were performed as described above under example 4 using the recombinant plant Lewis-type β1,3-galactosyltransferase except that 10 nmol of GnGn-pyridylamine (GnGn-PA) were used as the acceptor substrate. After 20 h of incubation the sample was analyzed by normal-phase and reverse-phase HPLC. FIG. 16 shows the measured enzyme activity of the recombinant Lewis-type β1,3-galactosyltransferase as well as of the negative control, when GnGn-pyridylamine was used as a substrate. Normal-phase and reverse-phase HPLC data are shown. A3Gn-PA/GnA3-PA standards were derived from bovine fibrin.

The presumed product peak eluting slightly after the substrate GnGn-PA was collected (FIG. 16B). By MALDI-TOF MS the product's mass was determined to be 1557.66 which is in good agreement with being A3Gn-PA/GnA3-PA (FIG. 16C). Upon digestion with β1,3-galactosidase (New England Biolabs) in 50 mM sodium citrate buffer of pH 4.5 for 20 h at 37° C. the glycan eluted with about the retention of GnGn-PA.

### Example 7

### Expression of a Recombinant Mammalian Lewis-Type β1,3-Galactosyltransferase in Leaves of A. thaliana

Expression of the murine β1,3-galactosyltransferase 1 (NP_{—}064679) was performed as described for the plant enzyme in Example 2. Murine β1,3-galactosyltransferase 1 was amplified from mouse genomic DNA using the primers 5′-TTTATCTAGATGGCTTCAAAGGTCTCCTGCC-3 (SEQ ID NO: 18) and 5′-TTCTGGATCCTAACATCTCAGATGCTTCTTG (SEQ ID NO: 19), subcloned into Topo vector and sequenced. Subsequently the entire open reading frame was excised from the Topo vector by XbaI/BamHI digestion and ligated into XbaI/BamHI digested binary plant expression vector pPT2. Trans-genic plants were prepared and analyzed as described for the plant Lewis-type β1,3-galactosyltransferase in Example 2. FIG. 24 shows the immunoblot analysis of transgenic A. thaliana lines (#1-#11) expressing the murine β1,3-GalT1 in leaves. The Lewis-A specific antibody produced a strong signal in the transgenic plants, which was not present in untransformed wildtype plants (Col-0) and showed the presence of increased levels of Lewis-A epitope.

### Example 8

### Analysis of A. thaliana Mutants with Reduced Amounts of Plant Lewis-Type β1,3-Galactosyltransferase

Gene silencing of plant Lewis-type β1,3-galactosyltransferase was performed using a hairpin construct to mediate efficient knockdown of SEQ ID NO 1 expression in A. thaliana plants. For this purpose a binary plant expression vector, which contains intron 2 of the A. thaliana β1,2-xylosyltransferase gene (Strasser et al., 2000, FEBS LETT. 472: 105-108) was generated. Genomic DNA was isolated from A. thaliana leaves using a GenElute Plant Genomic DNA Purification Kit (Sigma) and used as a template to amplify the intron 2 of the A. thaliana β1,2-xylosyltransferase gene with the primer combination 5′-ATCAGGGATCCACTGCACGGTATGCTCCTC-3 (SEQ ID NO: 20) and 5′-ATCGTGGTACCTAGCTGCGTCTGCAAAAAG-3′ (SEQ ID NO: 21). The resulting product was BamHI/KpnI digested and cloned into cloning vector puc18 to create p18XTI2. A 422 bp fragment from SEQ ID NO. 1 was amplified with primers 5′-TATATCTAGAAAAGTCCTCTTGTTAACTTGGAAC-3′ (SEQ ID NO: 22) and 5′-TATAAGATCTTAAACATCTTTAGGTTTCCTTC-3′ (SEQ ID NO: 23) and cloned into XbaI/BamHI digested vector p18XTI2. To amplify the antisense construct the primers 5′-TATAGGTACCTTAAACATCTTTAGGTTTCCTTC-3′ (SEQ ID NO: 24) and 5′-TATAGAATTCGGATCCAAAAGTCCTCTTGTTAACTTGGAAC-3′ (SEQ ID NO: 25) were used and the resulting PCR product was KpnI/EcoRI digested and cloned into the sense-intron fragment containing vector. The sense-intron-antisense cassette was removed from the cloning vector by XbaI/BamHI digestion and cloned into the binary plant expression vector pPT2. A schematic presentation of the resulting construct is shown in FIG. 27. The resulting binary plant expression vector, which harbours the Lewis-type β1,3-galactosyltransferase sense-intron-antisense cassette was transferred into Agrobacterium tumefaciens and transgenic plants were generated by floral dipping of wildtype A. thaliana plants as described in Example 2. Transgenic plants were selected on kanamycin and subsequently transferred to soil. Total RNA and total protein extracts were isolated from siliques and stems as described in Example 2. Reverse transcriptase reaction was carried out using oligodT primers and AMV-RT (Promega). A control PCR reaction was performed from cDNA to monitor the expression of A. thaliana core α1,3-fucosyltransferase B (FucTB) mRNA. As shown in FIG. 28A FucTB transcript can be detected in all tested lines (c: wild type control; transgenic lines 2, 7, 8, 9, 10, 11). To analyse changes in the expression of the Lewis-type β1,3-galactosyltransferase mRNA the same cDNA was amplified with primers SEQ ID NO: 13 and SEQ ID NO: 14. Clearly reduced amounts of transcripts can be detected in some transgenic lines (especially lines 2, 7 and 9)(FIG. 28B), which indicates a successful gene silencing of the target gene, Lewis-type β1,3-galactosyltransferase.

Analysis of the Lewis-A epitope expression was performed by immunoblots as described in Example 2. Instead of leaves, which do not produce detectable amounts of Lewis-A epitope a reduction was monitored in protein extracts from stems (shown in FIG. 29A) and siliques (FIG. 29B), which display significant amounts of Lewis-A carbohydrate structures in wildtype A. thaliana plants. Reduction of Lewis-A epitope formation was clearly visible in transgenic lines, which contain the Lewis-type β1,3-galactosyltransferase hairpin gene silencing construct.

### Example 9

### Assay for the Plant Lewis-Type β1,3-Galactosyltransferase Using a Human Glycoprotein as Acceptor Substrate

Human transferrin (Sigma-Aldrich) was converted to the GnGn-gly-coform by neuraminidase and β-galactosidase treatment as described (Bencurova et al., 2004, Glycobiology, 14: 457-466). The GnGn-transferrin was incubated with recombinantly expressed and purified plant Lewis-type β1,3-GalT and α1,4-fucosyltransferase to generate the Lewis-A epitope on transferrin. 2 μg of human GnGn-transferrin was first incubated overnight at 30° C. with 10 mM MnCl2, 0.1 M MES buffer pH 7.0, 0.5 mM UDP-galactose and purified A. thaliana Lewis-type β1,3-GalT. Control reactions were performed either without UDP-galactose or without acceptor substrate GnGn-transferrin. Subsequently, samples were incubated overnight with 2 μl of purified A. thaliana α1,4-fucosyltransferase in 10 mM MnCl2, 0.1 M MES buffer pH 7.0 and 0.5 mM GDP-fucose at 30° C. Samples were separated on 12% SDS-PAGE and blotted onto a ECL-membrane (Amersham Biosciences). The membrane was blocked with 3% (w/v) bovine serum albumin in PBST (phosphate buffer saline with 0.1% (v/v) Tween 20.) pH 7.4 and subsequently incubated for 16 h at 4° C. with JIM84 antibody as described in Example 2. As shown in FIG. 33 a specific signal on the transferrin can only be detected in lane 1, which contains all substrates. Lane 2 (no transferrin in the assay) and lane 3 (no UDP-galactose in the assay) show only unspecific bands.

### Example 10

### Identification and Sequences of Lewis-Type β1,3-GalT of Important Plants

1. rice (2× complete sequences)
2. poplar (1× complete)
3. maize (3× complete sequences)
4. tobacco (N. tabacum, partial sequence)
5. N. benthamiana (partial sequence)
6. apple (Malus domestica, partial sequence)
7. cotton (partial sequence)
8. common bean (Phaseolus vulgaris, partial sequence)
9. leafy spurge (partial sequence)
10. lettuce (partial sequence)
11. tomato (partial sequence)
12. grapewine (partial sequence)
13. wheat (partial sequence)
14. potato (partial sequence)
15. Medicago (partial sequence)

>Ricel
SEQ ID NO 31:
NKKWHGGFVIVSLFIILMLRYVILDSPLAEKSLQYVFQQNNTTPLHWLDV
PNPPAVQNPQNISQVISTELLASNLSITRNFSDRELQSLHSWNHLKDLVS
HAHILPDGVEAIKEAGVAWRELNAALEYDESAVSVNGSTHQKSKEKQCPY
SIRRMNATRSGDRFVLKIPCGLIQGSSITIIGTPGGLLGSFKIELTGATV
PGEPDPPIVLHYNVRLLGDKLTEDPVIVQNTWTIADDWGSEDRCPSPGSD
AKDIAKVDELEKCGSMVGNDQKQAWATKLKSNVSSIQPAWKKNTEPKKYF
PFRQGYLAIAILRVGAHGIHMTVDGKHVTSFAFREDLEPGFVGEVRIAGD
ILKKSVIASGLPTTEDFEHVTDLEILKAPPVPMDKPLDLFIGIFSTANNF
KRRMAVRRTWMQYDAVRSGKAAVRFFVGLHKNEVVNEELWNEARTYGDIQ
LMPFVDYYSLILWKTIAICIYGTNVLSAKYVMKTDDDAFVRVDEILLSLD
RVNISHGLLYGRVNSDSQPHRDPYSKWYITPEEWPEESYPPWAHGPGYIV
SQDIAEKVYRKHKRGELKMFKLEDVAMGIWINEMKKGDLDVKYENDGRIL
VEGCEEGYVVAHYQEPRDMMCLWDKFLKTKRGTCCKE
SEQ ID NO 32:
ATGAAGAAATGGCACGGTGGTTTTGTCATAGTATCTTTGTTCATTATATT
GATGCTAAGATATGTTATATTGGATAGTCCACTTGCTGAAAAATCTCTTC
AGTATGTCTTCCAACAGAATAACACAACCCCACTACATTGGCTGGATGTT
CCAAACCCGCCTGCAGTTCAGAATCCACAGAACATCTCTCAAGTAATATC
AACTGAATTGTTAGCTTCAAACCTTTCCATAACAAGAAATTTCTCCGATA
GAGAACTGCAGTCATTGCATTCTTGGAATCATTTAAAAGACCTAGTAAGT
CATGCGCACATCTTACCAGATGGAGTAGAAGCAATCAAGGAAGCTGGAGT
AGCATGGAGGGAGCTAAATGCAGCCCTTGAATACGATGAATCTGCTGTTT
CTGTCAATGGTAGCACTCATCAAAAAAGTAAAGAGAAGCAATGCCCATAT
TCCATTCGAAGAATGAATGCTACAAGATCAGGTGATAGATTTGTCTTAAA
GATTCCTTGTGGACTGATTCAGGGCTCTTCAATAACTATTATCGGTACTC
CTGGTGGTCTTCTTGGTAGTTTTAAGATAGAATTAACTGGGGCTACTGTA
CCTGGTGAACCAGACCCTCCAATTGTGCTTCATTACAATGTCCGTCTTCT
TGGTGATAAACTTACAGAAGATCCTGTAATAGTCCAAAATACATGGACTA
TAGCTGACGACTGGGGTTCTGAAGATCGTTGCCCATCTCCTGGTTCAGAT
GCTAAGGACATTGCAAAAGTGGATGAGTTAGAAAAATGCGGTAGCATGGT
GGGCAATGATCAAAAACAAGCATGGGCCACTAAGTTAAAGTCCAATGTTT
CAAGCATACAACCTGCCTGGAAAAAGAACACAGAACCTAAGAAATATTTT
CCCTTCAGACAAGGATATCTTGCTATAGCAATTCTTCGTGTTGGAGCACA
TGGGATCCATATGACTGTAGATGGGAAACATGTCACTTCATTTGCCTTTC
GGGAGGATTTGGAGCCTGGATTTGTTGGTGAAGTAAGAATTGCAGGAGAT
ATTAAATTGTTGTCTGTGATAGCAAGTGGTTTGCCTACAACAGAGGACTT
TGAACATGTGACTGACTTAGAAATTTTGAAGGCTCCACCAGTTCCCATGG
ACAAACCCCTGGATCTTTTTATTGGGATCTTTTCTACAGCTAACAATTTC
AAGCGCAGAATGGCAGTTCGCAGAACTTGGATGCAGTATGATGCTGTCCG
CTCAGGAAAGGCTGCAGTTCGATTCTTTGTTGGCCTGCATAAAAATGAGG
TGGTGAATGAGGAACTTTGGAATGAAGCACGAACATATGGAGACATCCAA
TTGATGCCCTTTGTTGATTATTATAGCCTGATTCTATGGAAGACTATAGC
CATCTGCATCTATGGGACAAACGTCTTGTCAGCCAAGTATGTGATGAAAA
CAGATGATGATGCTTTTGTCCGTGTGGATGAGATACTTTTATCTTTAGAT
CGCGTGAACATCAGTCATGGGTTACTGTACGGTCGTGTCAATTCTGATTC
TCAGCCTCATCGAGACCCATATAGCAAGTGGTACATAACCCCAGAGGAAT
GGCCTGAGGAGAGTTATCCTCCATGGGCACATGGTCCGGGATACATTGTA
TCACAAGACATAGCAAAAGAAGTTTACAGGAAACACAAGAGAGGAGAACT
TAAGATGTTCAAACTAGAAGATGTCGCAATGGGAATATGGATCAATGAGA
TGAAGAAGGATGGGTTGGATGTAAAGTATGAAAATGATGGGAGGATTTTG
GTCGAAGGCTGTGAGGAAGGATATGTGGTTGCACATTACCAGGAGCCAAG
GGACATGATGTGCCTATGGGACAAATTTCTGAAAACAAAACGAGGAACAT
GTTGCAAGGAGTAA
>Rice2
SEQ ID NO 33:
MWVTKRLGITVLIVLFPLLIVHHLIVNSPVSGPSRYQVIHSNLLGWLSDS
LGNSVAQNPDNTPVEVIPADASASNSSDSGNSSLEGFQWLNTWNHMKQLT
NISDGLPHANEAIDNARTAWENLTISVHNSTSKQIKKERQCPYSIHRMNA
SKPDTGDFTIDIPCGLIVGSSVTIIGTPGSLSGNFRIDLVGTELPGGSGK
PIVLHYDVRLTSDELTGGPVIVQNAFTASNGWGYEDRCPCSNCNNATQVD
DLERCNSMVGREEKRAINSKQHLNAKKDEHPSTYFPFKQGHLAISTLRIG
LEGIHMTVDGKHVTSFPYKAGLEAWFVTEVGVSGDFKLVSAIASGLPTSE
DLENSFDLAMLKSSPIPEGKDVDLLIGIFSTANNRKRRMAIRRTWMQYDA
VREGAVVVRFFVHLHTNLIVNKELWNEARTYGDIQVLPFVDYYSLITWKT
LAICIYGTGAVSAKYLMKTDDDAFVRVDEIHSSVKQLNVSHGLLYGRINS
DSGPHRNPESKWYISPEEWPEEKYPPWAHGPGYVVSQDIAKEINSWYETS
HLKMFKLEDVAMGIWIAEMKKGGLPVQYKTDERINSDGCNDGCIVAHYQE
PRHMLCMWEKLLRTNQATCCN
SEQ ID NO 34:
ATGTGGGTGACAAAGCGACTCGGAATTACGGTTCTAATCGTCCTCTTCCC
TCTGCTGATTGTGCATCATCTGATAGTGAATAGCCCAGTGTCAGGGCCAT
CTCGATACCAAGTCATACATTCTAATCTCCTTGGTTGGTTGAGCGACTCA
TTGGGAAATTCAGTGGCTCAAAATCCAGATAATACACCAGTAGAAGTGAT
ACCAGCAGATGCTTCAGCTTCAAACAGCTCGGATTCCGGAAATTCCTCTC
TTGAAGGATTTCAGTGGTTAAATACATGGAATCACATGAAGCAGTTGACC
AACATCTCCGATGGCCTTCCTCACGCAAATGAGGCTATAGACAATGCAAG
AACTGCATGGGAAAACCTGACGATATCAGTTCACAATTCAACCTCTAAAC
AGATTAAGAAAGAGAGGCAGTGTCCATATTCTATCCACAGAATGAATGCC
TCCAAGCCAGACACTGGAGATTTTACCATCGATATACCTTGTGGGCTGAT
TGTAGGTTCTTCAGTAACTATTATAGGCACTCCAGGTAGCCTCTCCGGTA
ACTTCAGGATTGATCTTGTTGGGACAGAATTGCCAGGGGGATCTGGGAAA
CCCATCGTACTGCACTATGATGTTCGTCTAACAAGTGATGAACTTACTGG
AGGTCCAGTCATAGTGCAGAATGCCTTCACTGCAAGTAATGGCTGGGGTT
ATGAGGATAGATGTCCCTGCAGTAACTGTAATAATGCAACTCAAGTGGAT
GATTTGGAGCGATGTAATTCCATGGTGGGTAGAGAAGAGAAGAGAGCTAT
AAACTCAAAACAGCACCTGAATGCCAAGAAAGATGAACACCCAAGCACAT
ATTTTCCATTTAAGCAGGGACACCTTGCTATCTCAACTCTTCGGATAGGG
CTAGAAGGGATTCACATGACAGTTGATGGAAAACATGTTACCTCATTTCC
ATATAAAGCGGGCTTGGAAGCATGGTTCGTTACTGAAGTAGGAGTATCTG
GTGATTTCAAGCTAGTAAGTGCAATTGCAAGTGGCCTGCCCACATCAGAA
GACCTGGAGAACAGCTTCGATCTTGCAATGTTGAAGTCGTCACCTATTCC
AGAGGGAAAAGATGTGGACCTTTTGATTGGTATCTTTTCAACAGCAAACA
ACTTCAAACGAAGGATGGCGATCCGACGGACATGGATGCAATATGATGCG
GTGCGCGAAGGAGCAGTAGTGGTCCGTTTTTTCGTTGGCCTGCATACAAA
CCTCATTGTAAACAAGGAACTCTGGAATGAAGCACGCACATATGGCGACA
TTCAAGTGCTGCCCTTTGTTGATTACTACAGTCTGATAACATGGAAGACA
CTGGCAATATGCATCTACGGGACTGGTGCTGTATCAGCGAAATATCTGAT
GAAAACAGACGATGATGCTTTTGTCCGTGTGGACGAGATACACTCCTCAG
TAAAACAACTGAATGTCAGTCACGGCCTACTTTATGGTCGTATCAATTCT
GACTCAGGTCCTCACAGAAATCCTGAGAGCAAATGGTACATAAGCCCAGA
GGAATGGCCTGAAGAGAAATACCCACCATGGGCACACGGACCAGGATATG
TGGTATCACAAGACATTGCAAAGGAAATCAACAGTTGGTATGAAACAAGT
CATTTGAAGATGTTCAAACTAGAAGATGTAGCAATGGGTATATGGATTGC
TGAAATGAAGAAGGGTGGGTTACCTGTCCAATACAAAACAGATGAAAGAA
TTAACAGTGATGGCTGCAATGATGGGTGCATCGTTGCTCACTACCAAGAA
CCAAGGCACATGCTATGCATGTGGGAGAAACTCCTAAGGACAAATCAAGC
AACGTGCTGCAACTAA
>Poplar (Populus trichocarpa)
SEQ ID NO 35:
MKKWYGGVLVASLFMLLILRYGLLKNPIGGNILLNPSSNASKPLEWVHPA
VPPAVQNPETSSQVFSTDTIVSSLFAPRNISNEEHKSLQTWNLLKHLIDH
AQVFANGVEAIKEAGNAWSSLMASAEEERLSYTNESSSRKVKEKQCPHFL
NKMNATELDNSGYKLWLPCGLTQGSSITIISIPDGLLGNFRIDLTGEALP
GEPDPPIILHYNVRLHGDKITEDPVIVQNTWTVAHDWGEEERCPSPSPEK
IKKVDELDQCNKMVGRNDTRVTSMHSDHSRRSSLQEGTKARRYFPFKQGQ
LSVATLRVGMEGIQMTVDGKHITSFAYRETLEPWLVSEVRISGDLNLISV
VASGLPTSEDSEHAVDLEVLKSAPLSPKKTLDLFIGVFSTANNFKRRMAV
RRTWMQYAAVRSGAVAVRFFVGLHKNQIVNEELWNEARTYGDIQLMPFVD
YYNLITFKTLAICIFGTEVASAKYVMKTDDDAFVRVDEVLASLKRIKVSH
GLLYGLINSDSRPHRSTESKWYISPEEWSEETYPPWAHGPGYVVSRDIAK
AVYKRYKEGRLKMFKLEDVAMGIWIAEMKKEGLEVKYEMEGRVHNEGCRD
GYVVAHYQAPREMLCLWQKLQEGNVARCCGDR
SEQ ID NO 36:
ATGAAGAAGTGGTACGGTGGTGTTCTGGTTGCATCCTTGTTCATGTTACT
GATCCTTAGATATGGTCTCCTGAAAAATCCAATCGGAGGAAATATTCTGT
TGAATCCCTCCTCCAATGCAAGCAAGCCTCTTGAATGGGTGCATCCTGCT
GTTCCACCTGCAGTTCAAAATCCAGAGACTTCTTCTCAAGTTTTTTCTAC
AGATACCATAGTCTCCAGTCTGTTTGCTCCGAGAAATATTTCCAATGAAG
AGCACAAGTCTCTGCAGACATGGAATCTACTGAAACACTTGATTGATCAT
GCTCAGGTTTTCGCAAATGGAGTAGAAGCTATTAAGGAAGCTGGAAATGC
GTGGAGCAGCCTGATGGCTTCAGCAGAAGAGGAAAGACTTAGTTATACAA
ATGAAAGTTCAAGTAGGAAAGTGAAAGAGAAACAATGTCCTCACTTTCTT
AACAAAATGAATGCCACAGAGCTTGATAATAGTGGTTATAAATTGTGGCT
TCCTTGTGGCTTGACTCAGGGTTCGTCCATCACAATTATCAGCATTCCAG
ATGGTCTTCTCGGTAATTTTCGGATCGACTTAACCGGGGAAGCACTTCCA
GGGGAGCCTGATCCACCCATCATTTTACATTACAATGTTAGGCTTCATGG
TGATAAGATTACAGAGGATCCAGTAATAGTCCAAAACACCTGGACTGTGG
CTCATGACTGGGGTGAAGAGGAGCGTTGTCCTTCTCCATCTCCTGAAAAA
ATTAAGAAAGTGGACGAGTTGGATCAATGTAATAAGATGGTGGGCAGAAA
CGATACTCGGGTGACTAGCATGCATTCTGATCATTCGAGAAGGTCATCAC
TTCAGGAAGGAACTAAAGCAAGAAGATATTTTCCATTTAAGCAAGGTCAG
CTATCTGTCGCAACGCTTAGAGTGGGAATGGAAGGAATCCAGATGACTGT
TGATGGAAAGCACATAACATCCTTTGCATATCGTGAAACTTTGGAGCCAT
GGCTAGTTAGTGAAGTTAGGATTTCTGGGGACCTAAATCTAATTTCAGTT
GTGGCTAGTGGTCTACCTACATCAGAGGATTCAGAGCATGCAGTTGATCT
AGAAGTACTCAAATCAGCTCCTCTCTCTCCAAAAAAAACACTGGATCTCT
TTATTGGTGTCTTTTCTACGGCAAACAATTTTAAGCGGAGGATGGCTGTT
CGAAGAACATGGATGCAGTATGCTGCAGTGCGATCAGGGGCAGTTGCAGT
GCGCTTTTTTGTTGGTCTGCATAAAAATCAAATAGTGAATGAGGAACTCT
GGAATGAAGCACGGACATATGGAGACATACAGCTGATGCCTTTTGTTGAC
TACTATAACCTCATCACCTTTAAAACTTTAGCTATCTGCATATTTGGGAC
AGAGGTTGCATCAGCTAAGTATGTCATGAAGACAGATGACGATGCGTTTG
TTCGTGTGGATGAAGTGTTAGCTTCTTTAAAGAGGATCAAAGTGAGTCAT
GGGTTGCTCTATGGATTAATCAATTCAGACTCTCGGCCTCATAGGAGTAC
TGAAAGCAAGTGGTATATTAGCCCTGAGGAATGGTCTGAAGAGACCTATC
CCCCCTGGGCACATGGTCCTGGTTACGTTGTGTCTCGTGACATTGCCAAG
GCAGTTTACAAGAGATACAAAGAAGGCCGTTTAAAGATGTTTAAGCTAGA
GGATGTAGCAATGGGTATCTGGATTGCTGAGATGAAAAAGGAGGGTTTAG
AAGTCAAATATGAGATGGAAGGGAGGGTCCATAATGAAGGGTGCAGGGAT
GGCTATGTTGTAGCCCACTACCAAGCTCCTAGGGAAATGCTCTGTCTGTG
GCAGAAACTTCAAGAAGGGAATGTTGCTAGATGCTGCGGTGATCGATAA
>Maizel
SEQ ID NO 37:
MKKWHGGFVIVSLLIILMLRYVILDRLLAEKSLQYVFQQNRTAELHWLDV
PNPPAIQNPQSSSEVISTRLLASNLSITRNLSDTELQSLHSWNHLKHLLT
HAHILPDGVEAIKEAGVAWRELNTALAYDDSVVSVNGSIQQKDKGKQCPY
SVRRMNATRLGDRGALKLPCGLIQGSSITIIGTPGGLLANFKIELTGAAV
PGEPDPPIVLHYNVRLLGDKLTEDPVIVQNTWTIADDWGSENRCPSPDSD
AKDSAKVDDLEKCSSMVGEDQKKILPSKLHSHVLTMPPARKKKAESRKYF
PFRQGYLAVAILRIGHAGIHMTVDGKHTISFAFREDLEPGFVGEVRIAGD
IKLLSVIASGLPTTEYFEHVTDLEILKAPPVAMDKSVDLFIGVFSTANNF
KRRMAVRRTWMQYDAVRSGKVAVRFEVGLHKNEVVNEELWNEARTYGDIQ
LMPFVDYYSQILWKTIAICIYGTNVLSAKYVMKTDDDAFVRVDEILSSLD
RTNISHGLLYGRVNSDSQPHRDPYSKWYITPEEWPEENYPPWAHGPGYIV
SQDIAKEVYRKHKNGELKMFKLEDVAMGIWINEMKKDGLDVKYENDGRIL
VEGCEDYVIAHYQEPRDMMCLWDKFQKTKRGTCCKE
SEQ ID NO 38:
ATGAAGAAATGGCATGGAGGCTTTGTCATTGTATCTTTGCTCATCATCTT
GATGTTAAGATATGTGATATTGGATAGGCTACTTGCAGAAAAATCGCTTC
AGTATGTTTTCCAGCAAAACAGGACAGCAGAATTACATTGGTTGGATGTT
CCAAACCCACCTGCAATCCAGAATCCACAGAGTTCTTCTGAAGTAATATC
AACCAGACTCTTGGCATCCAACCTTTCCATTACCAGAAATCTCTCTGATA
CAGAACTTCAATCTTTACATTCTTGGAATCATTTGAAACACCTTTTAACG
CATGCCCATATCCTACCAGATGGAGTAGAGGCAATCAAGGAAGCTGGAGT
TGCATGGAGGGAACTCAATACAGCCCTTGCATATGATGATTCAGTTGTTT
CTGTTAATGGCAGCATTCAGCAGAAGGACAAAGGAAAGCAATGCCCTTAT
TCTGTCCGACGGATGAATGCCACAAGATTAGGGGATAGGTTTGCTTTAAA
ACTTCCATGTGGATTGATTCAGGGCTCTTCAATAACAATTATTGGTACTC
CTGGTGGTCTTCTGGCTAATTTTAAGATAGAGTTAACTGGAGCTGCAGTT
CCTGGTGAACCAGACCCTCCAATTGTCCTTCATTATAATGTCCGCCTTCT
TGGTGATAAACTCACAGAAGATCCTGTAATAGTCCAAAATACATGGACGA
TAGCTGATGATTGGGGTTCTGAAAACCGTTGCCCATCTCCTGATTCAGAT
GCTAAGGACAGTGCAAAAGTGGATGATCTAGAAAAGTGTAGTAGCATGGT
AGGTGAGGACCAGAAGAAAATCCTTCCCTCTAAGTTACATTCACATGTTT
TGACCATGCCACCTGCAAGGAAGAAGAAAGCAGAATCTAGAAAGTATTTT
CCATTCAGGCAGGGATATCTTGCTGTAGCAATTCTTCGTATCGGAGCACA
TGGGATCCATATGACTGTAGATGGCAAACATATCACTTCGTTTGCATTTC
GAGAAGATCTGGAACCAGGGTTTGTTGGTGAAGTAAGAATTGCAGGAGAC
ATTAAATTGCTATCTGTGATAGCAAGTGGCCTGCCAACAACAGAGTACTT
CGAGCATGTCACTGATTTGGAAATTTTGAAGGCTCCACCTGTTGCCATGG
ATAAATCTGTTGATCTTTTCATTGGGGTCTTCTCTACAGCAAATAATTTC
AAACGCCGAATGGCAGTTCGCAGAACTTGGATGCAGTATGATGCTGTGCG
CTCAGGAAAAGTTGCAGTTCGATTCTTTGTTGGCCTGCACAAAAACGAGG
TGGTCAATGAGGAACTCTGGAATGAAGCACGAACATATGGAGACATCCAG
TTGATGCCGTTTGTGGATTACTATAGCCAGATTCTTTGGAAGACCATAGC
GATCTGCATCTATGGGACAAATGTCCTGTCAGCTAAGTATGTGATGAAAA
CTGATGATGATGCTTTTGTTCGAGTAGATGAAATACTTTCTTCTCTCGAC
CGAACCAATATCAGCCATGGACTGTTGTATGGTCGTGTCAATTCTGATTC
CCAGCCTCATCGAGATCCCTATAGCAAGTGGTACATAACCCCTGAGGAAT
GGCCTGAGGAGAATTATCCTCCATGGGCGCATGGTCCAGGATATATTGTT
TCACAGGACATAGCTAAAGAAGTTTACAGGAAACACAAGAATGGGGAATT
AAAGATGTTCAAGCTGGAGGATGTCGCGATGGGAATATGGATCAATGAGA
TGAAGAAGGATGGCTTGGATGTCAAGTATGAGAATGATGGGAGGATCTTG
GTTGAAGGATGCGAGGATGGGTATGTGATTGCTCACTACCAAGAACCAAG
GGATATGATGTGCCTCTGGGATAAGTTTCAGAAAACAAAACGAGGAACAT
GTTGCAAAGAATAA
>Maize2
SEQ ID NO 39:
MKKWHGGFVIVSLFIILMLRYVILDRLLAEKSLQYVFQQNRTAELHWLDV
PNPPAIQNPQSSSEVISTRLLASNLSITRNLSDTELQSLHSWNHLHKLLT
HAHILPDGVEAIKEAGVAWRELNTALAYDDSVVSVNGSIQQKDKGKQCPY
SVRRMNATRLGDRFALKLPCGLIQGSSITIIGTPGGLLINAKIELTGAAV
PGEPDPPIVLHYNVRLLGDKLTEDPVIVQNTWTIADDWGSENRCPSPDSD
AKDSAKVDDLEKCSSMVGEDQKEILPSKLHSHVLTMPPARKKKAESRKYF
PFRQGYLAVAILRIGAHGIHMTVDGHKITSFAFREDLEPGFVGEVRIAGD
IKLLSVIAGGLPTTEYFEHVTDLEILKAPPVAMDKSVDLFIGVFSTANNF
KRRMAVRRTWMQYDAVRSGKVAVRFFVGLHKNEVVNEELWMEARTYGDIQ
LMPFVDYYSLILWKTIAICIYGTNVLSAKYVMKTDDDAFVRVDEILSSLD
RTNISHGLLYGRVNSDSQPHRDPYSKWYITPEEWPEENYPPWAHGPGYIV
SQDIAKEVYRKHKNGELKMFKLEDVAMGIWINEMKKDGLDVKYENDGRIL
VEGCEDGYVIAHYQEPRDMMCLWDKFQKTKRGTCCKE
SEQ ID NO 40:
ATGAAGAAATGGCATGGAGGCTTTGTCATTGTATCTTTGTTCATCATCTT
GATGTTAAGATATGTGATATTGGATAGGCTACTTGCAGAAAAATCGCTTC
AGTATGTTTTCCAGCAAAACAGGACAGCAGAATTACATTGGTTGGATGTT
CCAAACCCACCTGCAATCCAGAATCCACAGAGTTCTTCTGAAGTAATATC
AACCAGACTCTTGGCATCCAACCTTTCCATTACCAGAAATCTCTCTGATA
CAGAACTTCAATCTTTACATTCTTGGAATCATTTGAAACACCTTTTAACG
CATGCCCATATCCTACCAGATGGAGTAGAGGCAATCAAGGAAGCTGGAGT
TGCATGGAGGGAACTCAATACAGCCCTTGCATATGATGATTCAGTTGTTT
CTGTTAATGGCAGCATTCAGCAGAAGGACAAAGGAAAGCAATGCCCTTAT
TCTGTCCGACGGATGAATGCCACAAGATTGGGGGATAGGTTTGCTTTAAA
ACTTCCATGTGGATTGATTCAGGGCTCTTCAATAACTATTATTGGTACTC
CTGGCGGTCTTCTGGCTAATTTTAAGATAGAGTTAACTGGAGCTGCAGTT
CCTGGTGAACCAGACCCTCCAATTGTCCTTCATTATAATGTCCGCCTTCT
TGGTGATAAACTCACAGAAGATCCTGTAATAGTCCAAAATACATGGACGA
TAGCTGATGATTGGGGTTCTGAAAATCGTTGCCCATCTCCTGATTCAGAT
GCTAAGGACAGTGCAAAAGTGGATGATCTAGAAAAGTGTAGTAGCATGGT
AGGTGAGGACCAGAAGGAAATCCTTCCCTCTAAGTTACATTCACATGTTT
TGACCATGCCACCTGCAAGGAAGAAGAAAGCAGAATCTAGAAAGTATTTT
CCATTCAGGCAGGGATATCTTGCTGTAGCAATTCTTCGTATCGGAGCACA
TGGGATCCATATGACTGTAGATGGCAAACATATCACTTCGTTTGCATTTC
GAGAAGATCTGGAACCAGGGTTTGTTGGTGAAGTAAGAATTGCAGGAGAC
ATTAAATTGCTATCTGTGATAGCAGGTGGCCTGCCAACAACAGAGTACTT
CGAGCATGTCACTGATTTGGAAATTTTGAAGGCTCCACCTGTTGCCATGG
ATAAATCTGTTGATCTTTTCATTGGGGTCTTCTCTACAGCAAATAATTTC
AAACGCCGAATGGCAGTTCGCAGAACTTGGATGCAGTATGATGCTGTGCG
CTCAGGAAAAGTTGCAGTTCGATTCTTTGTTGGCCTGCACAAAAACGAGG
TGGTCAATGAGGAACTCTGGAATGAAGCACGAACATATGGAGACATCCAG
TTGATGCCATTTGTGGATTACTATAGCCTGATTCTTTGGAAGACCATAGC
GATCTGCATCTATGGGACAAATGTCCTGTCAGCTAAGTATGTGATGAAAA
CTGATGATGATGCTTTTGTTCGAGTAGATGAAATACTTTCTTCTCTCGAC
CGAACCAATATCAGCCATGGACTGTTGTATGGTCGTGTCAATTCTGATTC
CCAGCCTCATCGAGATCCCTATAGCAAGTGGTACATAACCCCTGAGGAAT
GGCCTGAGGAGAATTATCCTCCATGGGCGCATGGTCCAGGATATATTGTT
TCACAGGACATAGCTAAAGAAGTTTACAGGAAACACAAGAATGGGGAATT
AAAGATGTTCAAGCTGGAGGATGTCGCGATGGGAATATGGATCAATGAGA
TGAAGAAGGATGGCTTGGATGTCAAGTATGAGAATGATGGGAGGATCTTG
GTTGAAGGATGCGAGGATGGGTATGTGATTGCTCACTACCAAGAACCAAG
GGATATGATGTGCCTCTGGGATAAGTTTCAGAAAACAAAACGAGGAACAT
GTTGCAAAGAATAA
>Maize3
SEQ ID NO 41:
MKKWHGGFVIVSLFIILMLRYVILDNPLVENSLQYVFQQNRTAELHWLDV
PNPPAIQNLQISSEVISTGLLASNLSITRNLSDRELQSLHSWNHLKQLLS
HVHILPDGVEAIKEAGVAWRELNTALAYDDAVSVNGSIQQKDKGKQCPYS
IRRMNATRLGDRFALKLPCGLIQGSSITIIGTPGGLLGNFKIELTGAAVP
GEPDPPIVLHYNVRLLGDKLTEDPVIVQNTWTISDDWGSENRCPSPDSAD
KDTTKVDDLEKCSSMVGEDQKEILPSKFHSNVSAMPPASKKKAEPRKYFP
FRQGYLAVAILRIGAHGIHMTVDGHKITSFAFREDLEPGFVGEVRIAGDI
KLLSVIASGLPTTEDFEHVTDLEILKAPPVPMDKPVDLFIGVFSTANNFK
RRMAVRRTWMQYDSVRSGKVTVRFFVGLHKNELVNEELWNEARTYGDIAL
MPFVDYYSLILWKTIAICIYGTNVLSAKYVMKTDDDAFVRVDEILSSLDR
TNISHGLLYGRVNSDSQPHRDPYSKWYITPEEWPEENYPPWAHGPGYIVS
KDIAKEVYRKHKSGELKMFKLEDVAMGIWINEMKKDGLDVKYENDGRILV
EGCEDGYVIAHYQEPRDMMCLWDQFQKTKRGTCCKE
SEQ ID NO 42:
ATGAAGAAATGGCATGGAGGCTTTGTCATTGTATCTTTGTTCATCATCTT
GATGTTAAGATATGTGATATTGGATAACCCACTTGTAGAAAACTCGCTTC
AGTATGTTTTCCAGCAAAACAGGACGGCAGAATTACATTGGTTGGATGTT
CCAAACCCACCTGCAATCCAGAATCTACAGATTTCTTCTGAAGTAATATC
AACTGGACTCTTGGCTTCCAACCTTTCCATTACCAGAAATCTCTCTGATA
GAGAACTTCAATCTTTACATTCTTGGAATCATTTGAAACAGCTATTATCT
CATGTTCATATCCTACCAGATGGAGTAGAGGCAATCAAGGAAGCTGGAGT
TGCATGGAGGGAACTCAATACAGCCCTTGCATATGATGATGCAGTTTCTG
TTAATGGCAGCATTCAGCAGAAGGACAAAGGAAAGCAATGCCCTTATTCT
ATCAGAAGGATGAATGCCACAAGATTAGGGGATAGGTTTGCTTTAAAACT
TCCCTGTGGACTGATTCAGGGCTCTTCAATAACTATTATTGGTACACCTG
GTGGTCTTCTTGGTAATTTTAAGATAGAGTTAACTGGGGCTGCAGTTCCT
GGTGAACCAGACCCTCCAATTGTCCTTCATTATAATGTCCGCCTTCTTGG
TGATAAACTCACAGAAGATCCTGTAATAGTCCAAAATACATGGACAATAT
CTGATGATTGGGGTTCTGAAAACCGTTGCCCATCTCCTGATTCAGATGCT
AAGGACACTACAAAAGTGGATGATCTAGAAAAATGTAGTAGCATGGTAGG
TGAGGACCAGAAAGAAATCCTTCCCTCTAAGTTTCATTCAAATGTTTCGG
CCATGCCACCTGCAAGTAAGAAGAAAGCAGAACCTAGAAAGTATTTTCCA
TTCAGACAGGGATATCTTGCCGTAGCAATTCTTCGGATCGGAGCACATGG
GATCCATATGACTGTAGATGGCAAACATATCACTTCTTTTGCATTCCGAG
AGGATTTGGAACCAGGGTTTGTAGGTGAAGTAAGAATTGCAGGAGACATT
AAATTGTTATCTGTGATAGCAAGTGGCCTGCCTACAACAGAGGACTTCGA
GCATGTCACTGACTTAGAAATATTGAAGGCTCCACCTGTGCCCATGGATA
AACCGGTTGATCTTTTCATTGGGGTCTTCTCTACAGCAAATAATTTCAAA
CGCCGAATGGCAGTTCGCAGAACTTGGATGCAGTATGATTCTGTTCGATC
AGGAAAAGTTACAGTTCGATTCTTTGTTGGCCTGCACAAAAATGAGTTGG
TCAATGAGGAACTCTGGAATGAAGCACGAACATATGGAGACATCCAGTTG
ATGCCATTTGTGGATTACTATAGCCTGATTCTTTGGAAGACCATAGCGAT
CTGCATCTATGGGACAAATGTCCTGTCAGCTAAGTATGTGATGAAAACTG
ATGATGATGCTTTTGTTCGAGTAGATGAAATACTTTCTTCTCTCGACCGA
ACCAATATCAGCCATGGACTGTTGTATGGTCGTGTCAATTCTGATTCCCA
GCCTCATCGAGATCCCTATAGCAAGTGGTACATAACCCCTGAGGAATGGC
CTGAGGAGAATTATCCTCCCTGGGCGCATGGACCAGGATATATTGTTTCA
AAGGACATAGCTAAAGAAGTTTACAGGAAACACAAGAGTGGGGAGTTAAA
GATGTTCAAGCTGGAGGATGTCGCAATGGGAATATGGATCAATGAGATGA
AGAAGGATGGCTTGGATGTCAAGTATGAGAATGATGGGAGGATTTTGGTT
GAAGGATGCGAGGATGGCTATGTGATTGCTCATTACCAAGAACCAAGGGA
TATGATGTGCCTCTGGGATCAGTTTCAGAAAACAAAACGAGGAACATGTT
GCAAAGAATAA
>Nicotiana tabacum
SEQ ID NO 43:
VSDLFIKRNLSAQEQQSLSTWYQLKRLVTHDQVLPNAIEAVKEASVAWNN
LMSAVEREKLDANDSSVKTGKQKQCPHFLSKTNATEIDASGFKLRFPCGL
TQGSSITIIGIPNGLLGNFRIDLTGEPLPGEPDPPVILHYNVRLHGDKIT
EDPVIVQNTWTIAHSWGEEERCPLPSDEKSKKVDELEQCNEMVGNVITRH
VIATNKSSLAQDGSKSRKYFPFKQGYLSVATLRVGSEGIQMAVDGKHITS
FAFRETLEPWLVSEVRISGDIKLISVVASGLPTSEDSEHISDLEALKAVP
LPPRKRLDLFVGVFSTANNFKRRMAVRRTWMQYDAVRSGQVAVRFFVGLH
KNQMVNEELWNEARTYMDIQLMPFVDYYSLIAWKTIAICVFGTEVISAKF
VMKTDDDAFVRVDEILSSMQRINVTRGLLYGLINSDSHPHRSPDSKWFIS
PEEWPEETYPPWAHGPGYVVSSDIAKTISSKQRKGRLKMFKLEDVAMGIW
ISEIKKKGLEVKYEKEERIFNEGCRDGYVIAHYQGPREMLCLWQKIEEKK
RALCCGE
SEQ ID NO 44:
GTCTCTGATCTCTTTATCAAGAGGAACCTGTCAGCTCAGGAGCAACAATC
TTTGTCTACGTGGTATCAGTTGAAACGCTTAGTTACTCATGATCAAGTCT
TACCTAATGCTATAGAGGCAGTTAAGGAAGCCAGTGTTGCATGGAACAAC
CTCATGAGTGCAGTAGAGAGGGAAAAACTTGATGCAAATGATAGTTCAGT
TAAGACCGGGAAACAGAAACAATGCCCTCATTTTCTCAGCAAAACCAATG
CTACAGAAATTGATGCCAGTGGCTTTAAGTTGCGGTTTCCTTGTGGTCTG
ACTCAGGGTTCTTCCATCACAATAATTGGCATTCCAAATGGTCTTCTTGG
GAATTTTCGGATAGATTTGACTGGTGAACCACTTCCAGGTGAACCAGATC
CTCCTGTTATTCTGCATTACAATGTTAGGCTCCATGGTGATAAAATAACT
GAGGATCCCGTAATTGTACAAAACACCTGGACAATTGCACATGACTGGGG
TGAAGAGGAGCGCTGTCCCTTACCATCGGATGAAAAGAGTAAGAAAGTGG
ATGAATTGGAGCAATGCAATGAGATGGTGGGAAATGTCATAACTCGACAT
GTCATTGCAACAAATAAATCTAGCCTGGCTCAGGACGGGTCTAAATCAAG
AAAATATTTTCCtTTCAAGCAAGGATATCTCTCTGTTGCCACTCTGAGAG
TAGGATCTGAAGGAATTCAGATGGCAGTTGATGGAAAACACATAACATCT
TTTGCTTTCCGTGAAACTTTGGAACCGTGGCTTGTAAGTGAAGTGAGGAT
ATCCGGAGACATAAAATTAATTTCTGTTGTCGCGAGTGGTTTGCCAACAT
CTGAGGATTCAGAGCATATAAGTGACTTGGAAGCTCTAAAAGCAGTTCCT
CTTCCTCCTCGGAAAAGACTAGATCTCTTTGTTGGTGTATTTTCTACCGC
AAATAATTTTAAGCGCAGAATGGCTGTCCGTAGAACTTGGATGCAATATG
ATGCAGTGCGATCTGGACAAGTTGCGGTGCGGTTTTTTGTTGGCTTGCAT
AAAAACCAAATGGTGAATGAAGAGCTCTGGAATGAGGCTAGGACATATAT
GGACATCCAGCTGATGCCTTTTGTTGATTACTATAGTCTTATCGCTTGGA
AGACCATTGCCATATGTGTTTTTGGGACGGAGGTCATTTCGGCAAAGTTT
GTCATGAAGACAGATGATGACGCATTTGTTCGAGTGGATGAAATCTTGTC
TTCTATGCAGAGGATTAATGTGACTCGCGGATTGCTCTATGGTCTTATCA
ACTCAGATTCCCATCCTCATAGGAGCCCAGACAGCAAGTGGTTTATCAGT
CCAGAGGAATGGCCTGAAGAAACTTACCCTCCTTGGGCACATGGACCGGG
TTATGTTGTGTCCAGTGATATAGCAAAAACAATCAGCTCAAAACAGAGAA
AAGGCCGCCTAAAGATGTTTAAGCTGGAAGATGTTGCTATGGGCATCTGG
ATTTCAGAAATAAAGAAGAAAGGGTTGGAAGTGAAGTATGAAAAAGAAGA
GAGGATTTTTAATGAAGGTTGCCGAGATGGTTATGTTATTGCACATTACC
AAGGCCCTAGAGAGATGCTCTGTCTTTGGCAAAAGATTGAAGAGAAAAAA
CGGGCTTTATGTTGTGGCGAGTAA
>Nicotiana benthamiana
SEQ ID NO 45:
SAQEQQSLSTWYQLKRLVTHDRVLPNAIEAVKEASIAWNNLMSAVEREKL
DANDTSVKTGKQKQCPHFLSKTNATEIDASGFKLRFPCGLTQGSSITIIG
IPNGLLGNFRIDLTGEPLPGEPDPPVILHYNVRLHGDKITEDPVIVQNTW
TIAHDEGEEERCPLPSDEKSKKVDELEQCNEMVGNIMATRHVIATNKSSL
AQDRSKSRKYFPFKQGYLSVATLRVGSEGIQMTVDGKHITSFSFRETLEP
WLVSEVRISGDIKLISVVASGLPTSEDSEHISDLEALKAAPLPPRKRLDL
FVGVFSTANNFKRRMAVRRTWMQYDAVRSGQVAVRFFVGLHKNQMVNEEL
WNEARTYMDIQLMPFVDYYSLIAWKTIAIVFGGTEVVSAKFVMKTDDDAF
VRVDEILSSMQRINVTRGLLYGLINSDSHPHRSPDSKWFISPEEWPEETY
PPWAHGPGYVVSSDIAKQSAQNRKKGRLKMFKLEDVAMGIWISEMNKKGL
EVKYEKEERIFNEGCRDGYVIAHYQGPREMLCLWQKIEEKKRALCWGE.
SEQ ID NO 46:
TCAGCTCAGGAGCAACAATCTTTGTCTACGTGGTATCAGTTGAAACGCTT
AGTTACTCATGATCGAGTCTTACCTAATGCTATAGAGGCAGTTAAGGAAG
CCAGTATCGCATGGAACAACCTCATGAGTGCAGTAGAGAGGGAAAAACTT
GATGCAAATGATACTTCAGTTAAGACAGGGAAACAGAAACAATGCCCTCA
TTTTCTCAGCAAAACCAATGCTACAGAAATTGATGCCAGTGGCTTTAAGT
TGCGATTTCCTTGTGGTCTGACTCAGGGTTCTTCCATCACAATAATTGGC
ATTCCAAATGGTCTTCTTGGGAATTTTCGGATAGATTTGACTGGTGAACC
ACTTCCAGGTGAACCAGATCCTCCTGTTATTCTGCACTACAATGTTAGAC
TCCATGGTGATAAAATAACTGAGGATCCCGTAATTGTACAAAACACCTGG
ACAATTGCACATGACTGGGGTGAAGAGGAGCGCTGTCCCTTACCATCGGA
TGAAAAGAGTAAGAAAGTGGATGAATTGGAACAATGCAATGAGATGGTGG
GAAATATCATGGCTACTCGACATGTCATTGCAACAAATAAATCTAGCCTG
GCTCAGGACAGGTCTAAATCAAGAAAGTATTTTCCTTTCAAGCAAGGATA
TCTCTCTGTCGCCACTCTGAGAGTAGGATCTGAAGGAATTCAGATGACAG
TTGATGGAAAACACATAACATCTTTTTCTTTCCGTGAAACTTTGGAACCG
TGGCTTGTAAGTGAAGTGAGGATATCCGGAGACATAAAATTAATTTCTGT
TGTCGCAAGTGGTTTGCCAACATCTGAGGATTCAGAGCATATAAGTGATT
TGGAAGCTCTAAAAGCAGCTCCTCTTCCTCCTCGGAAAAGACTAGATCTC
TTTGTTGGTGTATTTTCTACCGCAAATAATTTTAAGCGCAGAATGGCTGT
CCGTAGAACTTGGATGCAATATGATGCAGTGCGATCTGGACAAGTTGCGG
TGCGGTTTTTTGTCGGCTTGCATAAAAACCAAATGGTGAATGAAGAGCTC
TGGAATGAGGCTAGGACATATATGGACATCCAGTTGATGCCTTTTGTTGA
TTACTATAGTCTTATCGCTTGGAAGACCATTGCCATATGTGTTTTTGGGA
CCGAGGTCGTTTCGGCAAAGTTTGTCATGAAGACAGATGATGACGCATTT
GTTCGAGTGGATGAAATCTTGTCTTCTATGCAGAGGATTAACGTGACTCG
CGGATTGCTCTATGGTCTTATCAACTCAGATTCCCATCCTCATAGGAGTC
CAGACAGCAAGTGGTTTATCAGTCCAGAGGAATGGCCTGAAGAAACTTAC
CCTCCTTGGGCACATGGACCGGGTTATGTTGTGTCCAGTGATATAGCAAA
ACAATCAGCTCAAAACAGAAAAAAAGGCCGCCTAAAGATGTTTAAGCTGG
AAGATGTTGCTATGGGCATCTGGATTTCAGAAATGAATAAGAAAGGGTTG
GAAGTGAAGTATGAAAAGGAAGAGAGGATTTTTAATGAAGGTTGCCGAGA
TGGTTATGTTATTGCACATTACCAAGGCCCTAGAGAGATGCTGTGTCTTT
GGCAAAAGATTGAAGAGAAAAAACGGGCTTTATGTTGGGGCGAGTAA
>Apple (Malus domestica)
SEQ ID NO 47:
GGLKLISVLASGLPTSEDSEHIINLQELKSAPLSPHKQMDLFIGVFSTAN
NFKRRVAVRRTWMQYAAVRSGAVSVRFFVGLHKNQIVNEELWKEAQTYGD
IQLMPFVDYYGLITWKTLAICIFGTEVVSAKFVMKTDDDAFVRVDEVLAS
LHRIKVNRGLLYGLINSDSRPHRSPDSKWYISNEEWPEATYPPWAHGPGY
VVSNDIAKAVSQRYKKGRLKMFKLEDVAMGIWIADMKKEGLNVRYEKEEK
VYNEGCKDGYVVAHYQGPREMLCLRQKIPKGQVA
SEQ ID NO 48:
GGAGGCCTGAAATTAATTTCTGTCCTAGCCAGCGGTTTACCCACGTCAGA
GGATTCAGAGCACATAATTAATTTACAAGAACTCAAATCTGCTCCTCTCT
CGCCTCACAAACAAATGGATCTCTTTATTGGTGTTTTCTCTACCGCAAAC
AATTTTAAACGACGGATGGCTGTTCGAAGAACATGGATGCAGTATGCTGC
TGTACGATCAGGGGCAGTTTCAGTGCGCTTTTTTGTTGGTTTGCATAAGA
ACCAAATAGTGAATGAGGAACTTTGGAAAGAGGCACAAACCTATGGAGAC
ATACAGCTGATGCCTTTTGTTGATTACTATGGCCTCATTACCTGGAAAAC
TCTGGCCATCTGCATCTTTGGGACGGAGGTTGTTTCAGCGAAGTTCGTTA
TGAAGACGGATGACGATGCATTTGTTCGTGTGGATGAAGTGCTAGCTTCC
TTACACAGGATCAAAGTGAATCGTGGCTTGCTCTATGGCCTCATTAACTC
AGATTCTCGACCTCATCGAAGTCCAGATAGCAAGTGGTATATCAGCAATG
AGGAATGGCCTGAAGCAACTTACCCACCTTGGGCGCACGGTCCTGGTTAT
GTGGTATCGAATGACATAGCAAAGGCAGTTTCCCAGCGATATAAAAAGGG
CAGATTAAAGATGTTTAAGCTGGAAGACGTGGCGATGGGTATCTGGATCG
CGGATATGAAAAAGGAGGGTCTAAACGTACGGTATGAGAAGGAAGAGAAG
GTCTACAACGAAGGATGCAAGGACGGGTATGTCGTTGCACATTACCAAGG
TCCCAGGGAGATGCTGTGTCTCCGGCAGAAAATTCCTAAAGGGCAAGTTG
CTTGA
>Cotton
SEQ ID NO 49:
DHTVDIEALLSVPLSLQRPVDLFIGVFSTANNFKRRMAVRRTWMQYPEVR
SGTVVVRFFVGLHKSHIVNDELWNEARTYGDIQLMPFVDYYSLITWKTLA
ICTFGTEVVSAKFVMKTDDDAFVRLDEVMASLSRINVTHGLLYGLINSDS
QPHRSTDSKWFISPEEWSEEKYPPWAHGPGYVVSHDIAKAVYKRFNEVRL
KMFKLEDVAMGIWIADMQKDGLEVHYEKEERIFNEGCKDGYVIAHYQGPR
EMMCLWQKLPGDQKS
SEQ ID NO 50:
GACCATACGGTTGATATAGAAGCACTCCTTTCAGTTCCTCTTTCTCTTCA
AAGACCAGTGGATCTCTTTATTGGTGTTTTCTCTACTGCCAATAATTTTA
AAAGAAGAATGGCTGTGAGAAGAACATGGATGCAGTATCCTGAAGTCCGA
TCTGGGACAGTTGTAGTGCGCTTTTTTGTTGGTTTGCATAAAAGCCATAT
AGTGAATGATGAACTCTGGAATGAAGCACGGACATATGGAGACATACAGC
TGATGCCTTTTGTTGATTACTACAGCCTGATCACCTGGAAAACCTTGGCT
ATTTGCACCTTTGGGACAGAAGTTGTTTCTGCAAAGTTTGTCATGAAGAC
AGATGATGATGCGTTTGTTCGTTTGGATGAAGTCATGGCTTCATTAAGCA
GGATAAATGTAACTCATGGATTGCTTTATGGACTCATTAACTCAGATTCC
CAACCTCATCGCAGTACAGATAGCAAGTGGTTTATTAGCCCTGAGGAATG
GTCTGAAGAGAAGTACCCACCTTGGGCGCATGGTCCTGGTTACGTGGTGT
CCCATGACATAGCAAAGGCTGTTTACAAGAGGTTCAATGAAGTGCGCTTA
AAGATGTTTAAACTCGAAGATGTGGCAATGGGCATTTGGATCGCGGATAT
GCAAAAAGATGGTTTAGAAGTCCATTATGAGAAGGAAGAGAGGATCTTTA
ACGAGGGTTGTAAGGATGGTTATGTTATTGCTCACTATCAAGGTCCAAGA
GAGATGATGTGTTTGTGGCAGAAACTCCCAGGAGACCAAAAGAGCTAG
>Common bean (Phaseolus vulgaris)
SEQ ID NO 51:
PFVDYYSLITWKTLAICLFGTQVSAKFVMKTDDDAFVRVDEVLGSLHRIN
VDHGLLYGLINSDSRPHRNTDSKWYISPEEWSEGTYPPWAHGPGYVVSHD
IARTVAKKFRENHLKMFKLEDVAMGIWIADMKKEGVDVRCENEDRVYPEG
CKDGYVVAHYQGPREMLCLWQKLEEGKGAKCCGDRK
SEQ ID NO 52:
CCATTTGTTGACTACTACAGCCTCATCACGTGGAAAACTTTGGCAATCTG
CCTTTTTGGGACACAAGTTTCGGCAAAGTTTGTCATGAAGACGGATGATG
ATGCGTTTGTCAGGGTAGATGAAGTTCTAGGCTCTTTGCACAGGATCAAT
GTTGATCATGGGTTACTGTATGGACTCATCAATTCGGATTCCAGACCTCA
TCGAAATACAGATAGCAAATGGTACATCAGCCCAGAGGAATGGAGCGAAG
GAACTTATCCTCCTTGGGCACATGGTCCCGGCTATGTGGTCTCACACGAT
ATAGCAAGAACGGTTGCCAAGAAATTCAGAGAGAACCATTTGAAGATGTT
TAAATTAGAAGACGTTGCAATGGGAATTTGGATTGCAGACATGAAGAAGG
AAGGTGTAGATGTTCGGTGTGAGAATGAGGACAGAGTGTATCCTGAGGGT
TGTAAGGATGGTTATGTGGTTGCCCATTACCAGGGCCCTAGGGAGATGCT
CTGTTTGTGGCAGAAACTTGAAGAAGGAAAAGGTGCAAAATGCTGTGGTG
ATAGGAAATGA
>Leafy spurge (Euphorbia esula)
SEQ ID NO 53:
TIDGKHITSFAYRETLEPWLVSEVRISGDMNLMSIVASGLPTSEDSEHAI
DLELLKSVPLPSKKTPPPHLFIGVFSTANNFKRRMAVRRTWMQYPHVRSG
TVAVRFFVGLHKNQLVNKELWNEASTYGDIQLMPFVDYYNLITWKTLAIC
IFGTEVASAKYVMKTDDDAFVRVDQVLASLKRIKVNRGLLYGLINSDSQP
HRSTDSKWYISLEEWPEEKYPPWAHGPGYVVSQDI
SEQ ID NO 54:
ACCATTGATGGAAAACACATCACATCTTTTGCGTATCGCGAGACATTGGA
GCCATGGCTGGTTAGCGAAGTTAGAATATCTGGAGACATGAATTTAATGT
CTATAGTGGCAAGTGGTCTACCTACATCAGAGGATTCAGAACATGCAATT
GATCTAGAGCTACTCAAATCGGTCCCTCTCCCCTCGAAGAAAACACCACC
ACCTCATCTCTTTATTGGAGTATTCTCCACAGCCAACAATTTTAAACGTA
GGATGGCTGTTCGAAGAACTTGGATGCAGTATCCTCATGTTCGCTCTGGG
ACTGTTGCTGTGCGCTTTTTTGTTGGGTTGCACAAAAATCAATTAGTTAA
CAAGGAACTTTGGAATGAAGCATCAACATATGGAGACATACAGCTTATGC
CTTTTGTGGACTATTATAACCTCATCACCTGGAAGACTTTAGCTATCTGC
ATCTTTGGGACTGAGGTTGCATCAGCAAAATATGTCATGAAGACAGATGA
TGATGCATTTGTTCGAGTGGATCAAGTTTTAGCTTCTTTAAAACGGATCA
AAGTGAATCGCGGCCTGCTTTATGGACTCATAAATTCAGATTCCCAGCCT
CATAGGAGTACTGATAGCAAATGGTATATTAGCCTTGAGGAATGGCCTGA
AGAGAAATATCCGCCATGGGCACATGGTCCTGGTTATGTGGTGTCTCAAG
ACATAGC
>Lettuce (Lactuca sativa)
SEQ ID NO 55:
MKKWYIYVLASASLLTLTILGYLIIQNPIREAYISSPPYFNTTNPLEWIN
SSSPPVQNQTPNPQIVSTDEIVSTLFIHRNISFNEQQPLHTWNHLRHLIS
HDRVLPNTVEAIKDGGIAWNNLMTTVEDDKRDMNRSSGGKKKEKQCPHFL
NKMNATELNDT
SEQ ID NO 56:
ATGAAGAAATGGTATATCTATGTCCTATCTGCATCTCTATTAACATTAAC
AATACTCGGGTACTTAATCATTCAAAATCCAATAAGAGAAGCTTACATTT
CATCACCTCCATACTTCAACACAACAAACCCTCTTGAATGGATAAATTCC
AGTTCCCCCCCTGTTCAAAACCAAACCCCCAACCCTCAAATTGTCTCCAC
TGATGAAATAGTCTCCACCCTCTTTATCCACAGAAACATCTCATTCAATG
AACAACAACCATTACACACATGGAACCATTTAAGACACTTAATAAGCCAT
GATCGTGTTCTTCCTAACACAGTTGAAGCAATCAAAGATGGTGGAATCGC
ATGGAATAACCTCATGACTACAGTTGAAGATGACAAACGTGACATGAATA
GAAGCTCAGGGGGGAAAAAGAAAGAGAAACAATGTCCCCATTTTCTTAAC
AAGATGAATGCCACAGAGCTTAATGATACT
>Tomato
SEQ ID NO 57:
TMLCVFHETLEPWLISEVRISGDIKLISVVASGLPTSEDSEHISDLEALK
AAPLPPRKRLDLFIGVFSTANNFKRRMAVRRTWMQYDAVRSGQVAVRFFV
GLHKNQMVNEELWNEARTYRDIQLMPFVDYYSLITWKTIAICVFGTEVVS
AKFVMKTDDDAFVRVDEILSSMERVNTTRGLLYGLINADSQPYRSPDSQW
FISPEEWSEET
SEQ ID NO 58:
ACGATGTTATGTGTCTTCCATGAAACTTTGGAACCATGGCTCATAAGTGA
AGTGAGGATATCGGGAGACATAAAATTAATTTCTGTTGTCGCAAGTGGTT
TACCAACATCAGAGGATTCAGAGCATATAAGCGACTTGGAAGCCTTAAAA
GCAGCTCCTCTTCCTCCACGAAAAAGACTAGATCTCTTTATTGGTGTATT
TTCTACAGCAAATAATTTTAAGCGTAGAATGGCTGTCCGTAGAACTTGGA
TGCAATATGATGCAGTGCGGTCTGGACAAGTTGCGGTGCGGTTTTTTGTT
GGCTTGCATAAAAACCAAATGGTGAATGAAGAGCTCTGGAATGAGGCTCG
GACATACAGAGACATCCAGCTGATGCCTTTTGTTGATTACTACAGTCTTA
TCACTTGGAAAACCATTGCCATCTGTGTTTTTGGGACGGAGGTTGTTTCT
GCAAAGTTTGTCATGAAGACAGATGATGATGCATTTGTTCGAGTGGATGA
AATCTTATCTTCTATGGAGAGGGTTAACACGACTCGTGGATTGTTATATG
GACTTATTAACGCAGATTCCCAACCTTACAGGAGTCCAGACAGCCAGTGG
TTTATCAGTCCAGAGGAATGGTCTGAAGAAACTTA
>Grapewine (Vitis vinifera)
SEQ ID NO 59:
GAVAVRFFVGLHKNQMVNEELWKEVQTYGDIQLMPVFVDYYSLITWKTIA
ICIFGTEAVSAKYVMKTDDDSFVRVDEVLASLKKTKVTHGLLYGLINSDA
RPHRSSDSKWYISPEEWAGETYPPWAHGPGYVVSNDIAKTVYKRHKEGHL
KMFKLEDVAMGIWIAEMKKGGMEVSYIKRREVYNEGCNDGYVV
SEQ ID NO 60:
GGGGCAGTTGCCGTGCGCTTTTTTGTTGGTCTGCATAAAAATCAGATGGT
GAATGAGGAACTCTGGAAGGAGGTGCAGACATATGGAGACATTCAGCTGA
TGCCTTTTGTTGACTATTACAGTCTTATAACCTGGAAGACCATAGCAATT
TGCATCTTTGGGACTGAGGCTGTTTCGGCCAAGTATGTGATGAAAACAGA
TGATGATTCATTTGTTCGTGTGGATGAAGTGCTTGCTTCTTTAAAGAAGA
CCAAAGTGACTCATGGGTTGCTGTATGGTCTTATTAACTCTGATGCTCGA
CCTCATCGTAGTTCTGACAGTAAATGGTACATCAGCCCTGAGGAATGGGC
AGGAGAAACTTATCCACCTTGGGCACATGGTCCTGGTTACGTGGTGTCAA
ATGACATAGCAAAAACAGTGTACAAGAGACACAAGGAAGGTCATTTAAAG
ATGTTCAAGCTGGAAGATGTAGCAATGGGGATCTGGATAGCAGAGATGAA
GAAGGGGGGAATGGAGGTTAGTTACATAAAAGAAGAGAGGGTCTATAATG
AGGGGTGCAACGATGGGTACGTTGTTG
>Wheat
SEQ ID NO 61:
AEPKKYYPFKQGNLAIAILRVGAEGIHMTVDGKHVTSFAFREDLEPGFVG
EVRIEGDIKLLSVLASGLPTTEDFEHVTDLEILAKAPPVPKDKSIDLFIG
IFSTANNFKRRMAVRRTWMQYDAVRSGKVAVRFFVGLHKNDVVNKELWNE
ARTYGDIQLMPFVDYYSLILWKTIAICIYGTNVLSAKYVMKTDDDAFVRV
DEILSSLHQVNIXHGLLYGRVNSDSQPHRDPY
SEQ ID NO 62:
gcagaacctaaaaaatattatcccttcaaacaaggaaatcttgctatagc
aattcttcgtgttggagcagaggggatccatatgactgtagatgggaaac
atgtcacttcctttgcatttcgagaggatttggagcctgggtttgttggt
gaagtaaggattgaaggagatattaaattgctgtctgtgctagcaagtgg
cttgcctacaacagaggactttgagcatgtcactgacttggaaatattga
aggctccacctgtccctaaggataaatccattgatctttttattgggata
ttctctacagcaaataattttaaacgcagaatggcggttcgaagaacctg
gatgcagtacgatgctgtccgctcaggaaaagtcgctgttcggttctttg
ttggcctgcataaaaatgatgtggtgaacaaggagctctggaacgaagca
cggacatatggagacatccaattgatgccatttgttgattattacagctt
gattctttggaagaccattgccatttgcatatatggaacaaatgtacttt
cagccaagtatgttatgaaaaccgacgatgacgctttcgttcgtgtagat
gagatactttcgtccctacatcaagtaaatatcanccatggactgctgta
tggtcgcgtcaattctgattctcagcctcaccgagatccgtat
>Potato
SEQ ID NO 63:
NNQVIPAEIVVSDLFIKRNLSAQEQQSLSTWYQLKRLITHDLVLPNAIEA
VKEATVAWNNLMSAVEMDKLDTNDSSIKTGKQKQCPHEFLSKTNATELDA
SGFKLRLPCGLTQGSSITIIGIPNGLLGNFRIDLTGEPLPGEPDPPVILH
YNVRLHGDKITEDPVIVQNTWTIAHDWGEKERCPLPSDEKSKKV
SEQ ID NO 64:
AATAATCAAGTAATACCTGCTGAAATTGTTGTCTCTGATCTCTTTATCAA
GAGGAACCTGTCAGCTCAGGAGCAACAATCTTTGTCTACGTGGTATCAAT
TGAAAAGGTTAATCACTCATGATCTAGTTTTACCTAATGCTATAGAGGCG
GTTAAGGAAGCCACTGTTGCATGGAACAACCTCATGAGTGCAGTAGAGAT
GGACAAACTCGATACAAATGATAGTTCAATTAAGACAGGGAAACAGAAAC
AATGTCCTCATTTTCTTAGCAAAACCAATGCTACAGAACTTGATGCTAGC
GGCTTTAAGTTGCGGCTTCCTTGTGGTCTGACTCAGGGTTCTTCCATCAC
AATAATTGGCATTCCAAATGGTCTTCTTGGGAATTTTCGGATAGATTTGA
CTGGTGAACCACTACCAGGTGAACCAGACCCTCCTGTTATTCTGCACTAC
AATGTTAGGCTCCATGGTGATAAAATAACCGAGGATCCTGTAATCGTACA
AAACACCTGGACTATTGCACATGACTGGGGTGAAAAGGAGCGCTGTCCAT
TACCATCCGATGAAAAGAGTAAGAAAGTGG

The class of Lewis-type β1,3-galactosyltransferase is highly divergent and nucleotide and amino acid sequences may vary considerably. The following sequence comparisons were performed with the EMBOSS Pairwise Alignment Algorithm (http://www.ebi.ac.uk/emboss/align/index.html) using the needle algorithm with the EBLOSUM62 Matrix, a Gap_penalty of 10.0 and a Extend_penalty of 0.5.

### 10.1. SEQ_ID_NO1 Compared to Poplar DNA

### Identity: 1340/1943 (69.0%)

### Similarity: 1340/1943 (69.0%)

### Gaps: 55/1943 (2.8%)

### 10.2. SEQ_ID_NO2 Compared to Poplar Amino Acid Sequence

### Identity: 415/645 (64.3%)

### Similarity: 503/645 (78.0%)

### Gaps: 15/645 (2.3%)

### 10.3. SEQ_ID_NO1 Compared to Rice1 DNA Sequence

### Identity: 1267/1972 (64.2%)

### Similarity: 1267/1972 (64.2%)

### Gaps: 98/1972 (5.0%)

### 10.4. SEQ_ID_NO2 Compared to Rice1 Amino Acid Sequence

### Identity: 379/647 (58.6%)

### Similarity: 486/647 (75.1%)

### Gaps: 14/647 (2.2%)

### 10.5. SEQ_ID_NO1 Compared to Rice2 DNA Sequence

### Identity: 1182/2002 (59.0%)

### Similarity: 1182/2002 (59.0%)

### Gaps: 206/2002 (10.3%)

### 10.6. SEQ_ID_NO2 Compared to Rice2 Amino Acid Sequence

### Identity: 329/656 (50.2%)

### Similarity: 423/656 (64.5%)

Gaps: 48/656 (7.3%)

1.-32. (canceled)

33. A method of expressing a plant Lewis-type β1,3-galactosyltransferase in an organism comprising:
providing the organism with a nucleic acid molecule comprising or complementary to a sequence A further defined as:
a) a sequence according to SEQ ID NO: 1 with an open reading frame from base pair 1-1932,
b) a sequence which is at least 50% identical with SEQ ID NO: 1,
c) a sequence which hybridizes with SEQ ID NO: 1 under stringent conditions; or
d) a sequence which has degenerated to SEQ ID NO: 1 due to the genetic code;
wherein the sequences a) to d) encode a plant protein having Lewis-type β1,3-galactosyltransferase activity; and
expressing a protein encoded by sequence A or if the organism comprises a sequence A in its wildtype form overexpressing a protein encoded by sequence A.

34. The method of claim 33, wherein the protein has a sequence of SEQ ID NO: 2 or is at least 50% identical with SEQ ID NO: 2.

35. The method of claim 33, wherein the protein comprises a galactosyltransferase domain according to SEQ ID NO: 4, or a galactosyltransferase domain which is at least 50% identical with SEQ ID NO: 4.

36. The method of claim 33, wherein expression and/or overexpression is facilitated by transfecting the organism with a nucleic acid comprising a sequence A or by upregulation of the expression of an endogenous sequence A.

37. The method of claim 33, wherein the nucleic acid molecule is a functional vector.

38. The method of claim 33, wherein the organism is a plant or plant cell.

39. A nucleic acid molecule comprising or complementary to a sequence A further defined as:
a) a sequence according to SEQ ID NO: 1 with an open reading frame from base pair 1-1932;
b) a sequence which is at least 50% identical with SEQ ID NO: 1;
c) a sequence which hybridizes with SEQ ID NO: 1 under stringent conditions; or
d) a sequence which has degenerated to SEQ ID NO: 1 due to the genetic code;
wherein the sequences a) to d) encode a plant protein having Lewis-type β1,3-galactosyltransferase activity and the nucleic acid molecule further comprises a disruption, deletion, insertion and/or substitution mutation such that it encodes an inactive Lewis-type β1,3-galactosyltransferase.

40. The nucleic acid molecule of claim 39, wherein the disruption, deletion, insertion and/or substitution mutation is in a galactosyltransferase domain according to SEQ ID NO: 4, or a galactosyltransferase domain which is at least 50% identical with SEQ ID NO: 4.

41. The nucleic acid molecule of claim 39, further defined as hybridizing to sequence A under stringent conditions.

42. The nucleic acid molecule of claim 41, further defined as hybridizing to SEQ ID NO: 1 under stringent conditions.

43. The nucleic acid molecule of claim 39, further defined as comprising a disrupted Lewis-type β1,3-galactosyltransferase gene, wherein the disruption is by insertion of an exogenous sequence into said gene such that the disruption prevents expression of functional β1,3-galactosyltransferase, wherein the gene, prior to disruption, coded for a plant Lewis-type β1,3-galactosyltransferase with an amino acid sequence of SEQ ID NO: 2 or with at least 50% identity to SEQ ID NO: 2.

44. A biologically functional vector comprising a nucleic acid molecule of claim 39 suitable to transfect an organism.

45. The biologically functional vector of claim 44, further defined as suitable to transfect a plant or a plant cell.

46. The biologically functional vector of claim 44, wherein the sequence is inversely oriented with respect to a promoter.

47. An RNA molecule further defined as a double-stranded siRNA molecule or microRNA, or artificial microRNA, comprising a base sequence of 10 to 100 bases or a double stranded hairpin-RNA comprising a base sequence of 10 to 1000 bases of SEQ ID NO: 1 or sequence A being complementary to SEQ ID NO: 1 or sequence A.

48. The RNA molecule of claim 47, further defined as a double-stranded siRNA molecule or microRNA, artificial microRNA, or double stranded hairpin-RNA of claim 47 comprising a base sequence of 15 to 40 bases of SEQ ID NO: 1, a sequence complementary to SEQ ID NO: 1, and/or sequence A.

49. The RNA molecule of claim 48, further defined as comprising a base sequence of 18 to 30 bases of SEQ ID NO: 1, a sequence complementary to SEQ ID NO: 1, and/or sequence A.

50. The RNA molecule of claim 47, further defined as a double stranded hairpin-RNA comprising a base sequence of 50 to 500 based of SEQ ID NO: 1, a sequence complementary to SEQ ID NO: 1, and/or sequence A.

51. The RNA molecule of claim 50, further defined as a double stranded hairpin-RNA comprising a base sequence of 100 to 300 based of SEQ ID NO: 1, a sequence complementary to SEQ ID NO: 1, and/or sequence A.

52. A method of reducing or preventing the expression of an endogenous plant Lewis-type β1,3-galactosyltransferase in an organism comprising:
transfecting the organism at least transiently with a nucleic acid molecule or vector comprising or complementary to a sequence A further defined as:
a) a sequence according to SEQ ID NO: 1 with an open reading frame from base pair 1-1932;
b) a sequence which is at least 50% identical with SEQ ID NO: 1;
c) a sequence which hybridizes with SEQ ID NO: 1 under stringent conditions; or
d) a sequence which has degenerated to SEQ ID NO: 1 due to the genetic code;
wherein the sequences a) to d) encode a plant protein having Lewis-type β1,3-galactosyltransferase activity and the nucleic acid molecule further comprises a disruption, deletion, insertion and/or substitution mutation such that it encodes an inactive Lewis-type β1,3-galactosyltransferase; or
a vector encoding such a nucleic acid; or
treating the organism with an RNA molecule further defined as a double-stranded siRNA molecule or microRNA, or artificial microRNA comprising a base sequence of 10 to 100 bases or a double stranded hairpin-RNA comprising a base sequence of 10 to 1000 bases of SEQ ID NO: 1 or sequence A being complementary to SEQ ID NO: 1 or sequence A; or
treating the organism with a nucleic acid molecule expressing such an RNA molecule or sequence A.

53. The method of claim 52, wherein the organism is a plant or a plant cell.

54. The method of claim 52, further defined as a method of producing a stably or transiently transfected plant or plant cell blocked or reduced expression of an endogenous Lewis-type β1,3-galactosyltransferase present in the wildtype plant or plant cell at the transcription or translation level.

55. The method of claim 54, wherein the reduction in expression is at least 10% as compared to the wildtype plant or plant cell.

56. The method of claim 54, wherein the plant or plant cell is capable of producing complex N-glycans after the transfection and an expression of a Lewis-A epitope is prevented by the transfection.

57. The method of claim 54, wherein a normal expression of the Lewis-A epitope is prevented in at least one organ or tissue, and the plant or plant cell comprises a dysfunctional β1,2-xylosyltransferase and/or a dysfunctional α1,3-fucosyltransferase.

58. The method of claim 54, wherein the plant or plant cell produces glycoproteins which lack β1,2-xylose and α1,3-fucose linked to complex N-glycans.

59. A transformed plant or plant cell with altered expression of a Lewis-type β1,3-galactosyltransferase, relative to expression in a non-transformed plant.

60. The transformed plant or plant cell of claim 59, further defined as having increased expression of a Lewis-type β1,3-galactosyltransferase, comprising a nucleic acid molecule of claim 33.

61. The transformed plant or plant cell of claim 59, further defined as having reduced or no expression of a Lewis-type β1,3-galactosyltransferase, comprising:
a nucleic acid molecule or vector comprising or complementary to a sequence A further defined as:
a) a sequence according to SEQ ID NO: 1 with an open reading frame from base pair 1-1932;
b) a sequence which is at least 50% identical with SEQ ID NO: 1;
c) a sequence which hybridizes with SEQ ID NO: 1 under stringent conditions; or
d) a sequence which has degenerated to SEQ ID NO: 1 due to the genetic code;
wherein the sequences a) to d) encode a plant protein having Lewis-type β1,3-galactosyltransferase activity and the nucleic acid molecule further comprises a disruption, deletion, insertion and/or substitution mutation such that it encodes an inactive Lewis-type β1,3-galactosyltransferase; or
a vector encoding such a nucleic acid; or
an RNA molecule further defined as a double-stranded siRNA molecule or microRNA, or artificial microRNA, comprising a base sequence of 10 to 100 bases or a double stranded hairpin-RNA comprising a base sequence of 10 to 1000 bases of SEQ ID NO: 1 or sequence A being complementary to SEQ ID NO: 1 or sequence A; or
an antisense nucleic acid to any sequence A.

62. The transformed plant or plant cell of claim 59, further defined as comprising a nucleotide sequence linked to an exogenous promoter that drives expression in said plant or plant cell wherein said nucleotide sequence encodes a functional mammalian galactosyltransferase that is expressed in the plant or plant cell.

63. The transformed plant or plant cell of claim 62, wherein the mammalian galactosyltransferase is a β1,4-galactosyltransferase.

64. The transformed plant or plant cell of claim 63, wherein the β1,4-galactosyltransferase is a human β1,4-galactosyltransferase.

65. The transformed plant or plant cell of claim 59, further defined as comprising a nucleotide sequence linked to an exogenous promoter that drives expression in said plant or plant cell wherein said nucleotide sequence encodes a functional mammalian N-acetylglucosaminyltransferase that is expressed in the plant or plant cell.

66. The transformed plant or plant cell of claim 65, wherein the mammalian N-acetylglucosaminyltransferase is a β1,2-N-acetylglucosaminyltransferase I or II, a β1,4-N-acetylglucosaminyltransferase III or IV, or a β1,6-N-acetylglucosaminyltransferase V.

67. The transformed plant or plant cell of claim 66, wherein the mammalian N-acetylglucosaminyltransferase is a human N-acetylglucosaminyltransferase.

68. The transformed plant or plant cell of claim 59, further defined as comprising a nucleotide sequence linked to an exogenous promoter that drives expression in said plant or plant cell, wherein said nucleotide sequence encodes a functional mammalian sialyltransferase that is expressed in the plant or plant cell.

69. The transformed plant or plant cell of claim 68, wherein the mammalian sialyltransferase is a human or rat α2,6-sialyltransferase or a human or rat α2,3-sialyltransferase.

70. The transformed plant or plant cell of claim 59, further defined as an alfalfa, Arabidopsis thaliana, maize, mung bean, potato, rice, soybean, tobacco, tomato plant, wheat, barley, Nicotiana benthamiana, lemna, lettuce, spinach, banana, sugarcane, poples, apple, cotton, common bean, leafy spurge, carrot, cowpea, papaya, Brassica, or grapewine plant.

71. A method of producing a recombinant mammalian glycoprotein or glycosylated polypeptide comprising:
obtaining a transformed plant of claim 59 that expresses a glycoprotein or glycosylated polypeptide; and
cultivating the plant to produce the recombinant mammalian glycoprotein or glycosylated polypeptide.

72. The method of claim 71, wherein the glycoprotein is a human antibody or a fragment thereof.

73. The method of claim 72, wherein the human antibody or a fragment thereof is a human immunoglobulin G (IgG) or a glycosylated fragment thereof.

74. The method of claim 71, wherein the glycoprotein or glycosylated polypeptide is insulin, preproinsulin, proinsulin, glucagon, an interferon, a blood-clotting factor, a fertility hormone, a growth factor, a granulocyte colony stimulating protein, prolactin, oxytocin, a thyroid stimulating hormone, an adrenocorticotropic hormone, calcitonin, a parathyroid hormone, a somatostatin, erythropoietin (EPO), hemoglobin, serum albumin, collagen, an enzyme, casein, whey protein, soya protein, gluten, or egg albumin.

75. The method of claim 74, wherein the glycoprotein or glycosylated polypeptide is alpha-interferon, beta-interferon, gamma-interferon, blood-clotting Factor VII, VIII, IX, X, XI, or XII, luteinizing hormone, follicle stimulating hormone, epidermal growth factor, platelet-derived growth factor, a beta-glucocerebrosidase amidase, an amylase, a carbohydrase, a cellulase, a dextranase, an esterase, a glucanase, a glucoamylase, a lactase, a lipase, pepsin, a peptidase, a phytase, a protease, or a pectinase.

76. The method of claim 74, wherein the glycoprotein or glycosylated polypeptide is a human glycoprotein or glycosylated polypeptide.

77. A method of producing a plant or a plant cell with increased plant Lewis-type β1,3-galactosyltransferase activity comprising introducing into the plant or plant cell a nucleic acid sequence encoding a plant Lewis-type β1,3-galactosyltransferase nucleotide sequence, mammalian β1,3-galactosyltransferase nucleotide sequence, or bryophyte Lewis-type β1,3-galactosyltransferase nucleotide sequence.

78. A transformed plant or plant cell with increased expression of the Lewis-A epitope, wherein the plant or plant cell comprises a mammalian β1,3-galactosyltransferase nucleotide sequence, a bryophyte Lewis-type β1,3-galactosyltransferase nucleotide sequence, or a plant Lewis-type β1,3-galactosyltransferase nucleotide sequence.

79. The transformed plant or plant cell of claim 78, further defined as comprising a β1,3-galactosyltransferase 1, β1,3-galactosyltransferase 2, or β1,3-galactosyltransferase 5 nucleotide sequence.
